(19) United States
(12) Patent Application Publication
(10) Pub. No.: US 2016/0008490 A1
(43) Pub. Date: Jan. 14, 2016

(54) METAL CHELATE COMPOUNDS FOR BINDING TO THE PLATELET SPECIFIC GLYCOPROTEIN IIB/IIIA

(71) Applicant: BAYER PHARMA AKTIENGESELLSCHAFT, Berlin (DE)

(72) Inventors: MARKUS BERGER, Berlin (DE); JESSICA LOHRKE, Berlin (DE); GREGOR JOST, Berlin (DE); MICHAEL REINHARDT, Berlin (DE)

(73) Assignee: BAYER PHARMA AKTIENGESELLSCHAFT, Berlin (DE)

(21) Appl. No.: 14/766,938

(22) PCT Filed: Feb. 11, 2014
(86) PCT No.: PCT/EP2014/052658

§ 371 Date: Aug. 10, 2015

Related U.S. Application Data
(60) Provisional application No. 61/764,159, filed on Feb. 13, 2013.

(30) Foreign Application Priority Data
Feb. 12, 2013 (EP) 13154880.2

(51) Int. Cl. A61K 49/14; C07F 5/00

(52) U.S. Cl. CPC A61K 49/14; C07F 5/003

(57) ABSTRACT

The present invention is directed to compounds that bind to glycoprotein IIb/IIIa and can be used for diagnostic imaging, in particular magnetic resonance imaging of thrombi. The disclosed compounds enable the binding to glycoprotein IIb/IIIa receptor combined with an adequate relaxivity.

### FIELD OF THE INVENTION

The present invention relates to the items characterized in the patent claims, namely metal chelates useful for magnet resonance imaging of thrombi and their use for imaging of thrombi in a mammalian body. More particularly, the invention relates to high-affinity, specific-binding glycoprotein IIb/IIIa antagonists labeled with paramagnetic chelates for imaging of thrombi.

### BACKGROUND

1. Introduction

Myocardial infarction (MI), stroke, transient ischemic attacks (TIA) and pulmonary embolism (PE) are major causes of morbidity and mortality worldwide. These life-threatening clinical events are mostly caused by thrombi, which can be located in different vessels spread all over the body and can be of different size and composition. The origin of stroke or TIA can for example be a thrombus in the left atrium (LA) of the heart or in one of the big arteries between heart and brain like the carotid artery. In case of PE a venous thrombosis, often situated in the lower legs, can be the cause.

In a growing thrombus the final common step of platelet aggregation is characterized by the binding of activated glycoprotein IIb/IIIa (GPIIb/IIIa) to blood fibrinogen resulting in a crosslinking inside the platelets. Design and development of glycoprotein IIb/IIIa inhibitors (Scarborough R. M., Gretler D. D., J. Med. Chem. 2000, 43, 3453-3473) has been of considerable interest in pharmacological research with respect to anti-platelet and anti-thrombotic activity.

However, health care professionals are in need not only for compounds that prevent thrombosis in an acute care setting, but also for a satisfactory method of imaging thrombi.

More particularly, thrombus imaging is of great importance for clinical applications such as thrombolytic intervention, in which the identification of the thrombus formation sites is essential for monitoring of therapy effects.

Thus thrombus imaging helps avoiding unnecessary prophylactic applications and therewith hazardous anticoagulant treatments (e.g. severe bleedings due to the reduced coagulation capacity).

The patient population which may benefit from such a diagnostic procedure is huge. According to the “Heart disease and Stroke Statistics—2010 Update” of the American Heart Association 17.6 million people suffered from coronary heart disease only in the USA. Every year an estimated 785,000 Americans will have a new coronary attack, and approximately 470,000 will have a recurrent attack. Every year about 795,000 patients experience a new or a recurrent stroke. About 610,000 of these are first attacks. Of all strokes, 87% are ischemic, most of them due to a thromboembolic cause (Lloyd-Jones, D. et al., Circulation, 2010, 121(7): p. e46-215). The incidence of transient ischemic attack (TIA) in the United States has been estimated to be approximately 200,000 to 500,000 per year, with a population prevalence of 2.3%, which translates into about 5 million people (Easton, J. D. et al., Stroke, 2009, 40(6): p. 2276-2293). Individuals who have a TIA have a 90-day risk of stroke of 3.0% to 17.3% and a 10-year stroke risk of 18.8%. The combined 10-year stroke, myocardial infarction, or vascular death risk is even 42.8% (Clark, T. G., M. F. G. Murphy, and P. M. Rothwell, Journal of Neurology, Neurosurgery & Psychiatry, 2003. 74(5): p. 577-580).

Imaging is forefront in identifying thrombus. Currently, thrombus imaging relies on different modalities depending on the vascular territory. Carotid ultrasound is used to search for carotid thrombus, transesophageal echocardiography (TEE) searches for cardiac chamber clot, ultrasound searches for deep vein thrombosis, and CT has become the gold standard for PE detection.

2. Description of the Prior Art, Problem to be Solved and its Solution

Despite the success of the above mentioned techniques, there is still a strong need for an imaging solution for thrombus detection and monitoring: first, there are certain vascular territories which are underserved. For instance, despite the best imaging efforts still 30% to 40% of ischemic strokes are “cryptogenic,” that is, of indefinite cause, or in other words, the source of the thromboembolism is still unfortunately not identified (Guercini, F. et al., Journal of Thrombosis and Hemostasis, 2008. 6(4): p. 549-554). Underlying sources of cryptogenic stroke include atherosclerosis in the aortic arch or intracranial arteries. Plaque rupture in the arch or other major vessels, in particular, is believed to be a major source of cryptogenic strokes and is very difficult to detect with routine methods. Recent clinical trial data from transesophageal Echocardiography (TEE) studies showed that the presence of thickened vessel wall in the aortic arch was not predictive of ischemic stroke, although ulcerated aortic arch plaques were associated with cryptogenic stroke. A thrombus-targeted specific imaging approach has a great potential to identify clots in the presence of atherosclerotic plaques.

Moreover, there is still a strong need for an approach wherein a single modality is used to identify thrombus throughout the body. For instance, in a TIA or stroke follow-up, currently multiple examinations are required to search for the source of the embolus (Ciesienski, K. L. and P. Caravan, Curr Cardiovasc Imaging Rep., 2010. 4(1): p. 77-84).

As already mentioned above the therapeutic application of glycoprotein IIb/IIIa inhibitors (Scarborough R. M., Gretler D. D., J. Med. Chem. 2000, 43, 3453-3473) has been of considerable interest in the past. Meanwhile three glycoprotein IIb/IIIa antagonists are commercially available: a recombinant antibody (Abciximab), a cyclic heptapeptide (Eptifibatid) and a synthetic, non-peptide inhibitor (Tirofiban). Tirofiban (brand name AGGRASTAT) belongs to the class of sulfonamides and is the only synthetic, small molecule among the above mentioned pharmaceuticals. Duggan et. al., 1994, U.S. Pat. No. 5,292,756 disclosed sulfonamide fibrinogen receptor antagonist as therapeutic agents for the prevention and treatment of diseases caused by thrombus formation.

Highly specific non-peptide glycoprotein IIb/IIIa antagonists have been described in the prior art (Damiano et. al., Thrombosis Research 2001 104, 113-126; Hoekstra, W. J., et al., J. Med. Chem., 1999, 42, 5254-5265). These compounds have been known to be GPIIb/IIIa antagonist, effective as therapeutic agents with anti-platelet and anti-thrombotic activity (see WO99/21832, WO97/41102, WO95/08536, WO96/29309, WO97/33869, WO9701/60813 and U.S. Pat. No. 6,515,130).

So far, there are only a few publications reporting on glycoprotein IIb/IIIa specific contrast agents for thrombus imaging. U.S. Pat. No. 5,508,020 describes radiolabeled peptides, methods and kits for making such peptides to image sites in a mammalian body labeled with technetium-99m via Tc-99m binding moieties. The SPECT tracer apticide (AcuTect®) is an approach to fulfill the need of thrombus imaging. Apticide is a Tc-99m labeled peptide, which specifically binds to the GPIIb/IIIa receptor. Dean and Lister-James describe peptides that specifically bind to GPIIb/IIIa receptors on the surface of activated platelets (U.S. Pat. No. 5,645,815; U.S. Pat. No. 5,830,856 and U.S. Pat. No. 6,028,056). The authors show the detection of deep vein thrombosis employing Apticide. However, the unspecific binding of the technetium labeled peptide and the low signal to noise ratio are the drawbacks of this method resulting a low resolution of the thrombus imaging. US 2007/0189970 describes compounds capable of binding to glycoprotein IIb/IIIa. The disclosed compounds are labeled with a positron emitting isotope or ¹¹C. In addition to nuclear medicine approaches for specific thrombus imaging, specific high relaxivity compounds which are useful for the diagnosis of many pathologies, in particular cardiovascular, cancer-related and inflammatory pathologies, are described in US 2006/0239926 A1.

Although the principle of associating a target specific binder (biovector) and a paramagnetic chelate has been known for quite some time, a specific MRI contrast agent has not yet been tested in clinical trials.

The targeting MRI approach does however present some difficulties. The main difficulty arises from the relatively low sensitivity of the MRI technique. Due to the intrinsically low sensitivity of MRI, high local concentrations of the contrast agent at the target site are required to generate detectable MR contrast. To meet this requirement, the specific MRI contrast agent has to recognize the target with high affinity and specificity. However, the steric effect of the paramagnetic chelates in comparison to the used small molecule GPIIb/IIIa binder can reduce the affinity for its target. In order to obtain an appropriate MRI thrombus imaging this problem has to be solved.

It has now been found, and this constitutes the basis of the present invention, that the compounds of the present invention have surprising and advantageous properties.

In particular, said compounds of the present invention have surprisingly been found to show a high affinity to platelet specific glycoprotein IIb/IIIa receptor and simultaneously have an adequate relaxivity for magnetic resonance imaging.

### SUMMARY

The present invention is directed to compounds that bind to glycoprotein IIb/IIIa and can be used for diagnostic imaging, in particular magnetic resonance imaging of thrombi. The disclosed compounds enable the binding to glycoprotein IIb/IIIa receptor combined with an adequate relaxivity.

### DESCRIPTION OF THE INVENTION

In accordance with a first aspect, the present invention covers compounds of general formula (I):

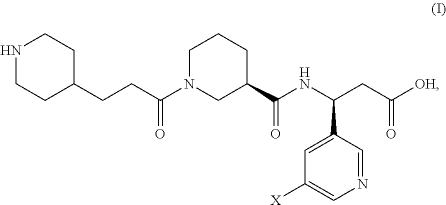

in which:

X represents a group selected from:

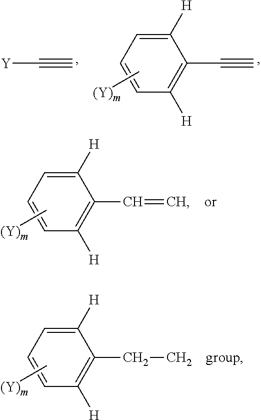

in which groups:

Y represents a:

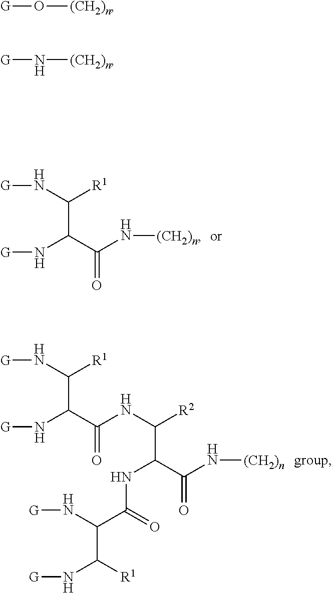

in which groups:

R¹ represents Hydrogen, Methyl, Ethyl, Propyl or iso-Propyl;

R² represents Hydrogen, Methyl, Ethyl, Propyl or iso-Propyl;

G represents a:

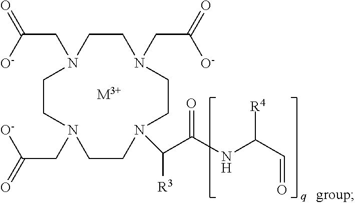

in which:

R³ represents Hydrogen, Methyl, Ethyl, Propyl, iso-Propyl or Benzyl;

R⁴ represents Hydrogen, Methyl, Ethyl, Propyl, iso-Propyl or Benzyl;

M represents Praseodymium, Neodymium, Samarium, Ytterbium, Gadolinium, Terbium, Dysprosium, Holmium or Erbium;

m represents 1 or 2;

n represents an integer of 2, 3, 4, 5 or 6;

q represents 0 or 1;

or a stereoisomer, a tautomer, an N-oxide, a hydrate, a solvate, or a salt thereof, or a mixture of same.

The compounds of this invention may contain one or more asymmetric centre, depending upon the location and nature of the various substituents desired. Asymmetric carbon atoms may be present in the (R) or (S) configuration, resulting in racemic mixtures in the case of a single asymmetric centre, and diastereomeric mixtures in the case of multiple asymmetric centres. In certain instances, asymmetry may also be present due to restricted rotation about a given bond, for example, the central bond adjoining two substituted aromatic rings of the specified compounds.

Preferred compounds are those which produce the more desirable biological activity. Separated, pure or partially purified isomers and stereoisomers or racemic or diastereomeric mixtures of the compounds of this invention are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., chiral HPLC columns), with or without conventional derivatisation, optimally chosen to maximise the separation of the enantiomers. Suitable chiral HPLC columns are manufactured by Daicel, e.g., Chiracel OD and Chiracel OJ among many others, all routinely selectable. Enzymatic separations, with or without derivatisation, are also useful. The optically active compounds of this invention can likewise be obtained by chiral syntheses utilizing optically active starting materials.

In order to limit different types of isomers from each other reference is made to IUPAC Rules Section E (Pure Appl Chem 45, 11-30, 1976).

The present invention includes all possible stereoisomers of the compounds of the present invention as single stereoisomers, or as any mixture of said stereoisomers, e.g. R- or S-isomers, or E- or Z-isomers, in any ratio. Isolation of a single stereoisomer, e.g. a single enantiomer or a single diastereomer, of a compound of the present invention may be achieved by any suitable state of the art method, such as chromatography, especially chiral chromatography, for example.

Further, the compounds of the present invention can exist as N-oxides, which are defined in that at least one nitrogen of the compounds of the present invention is oxidised. The present invention includes all such possible N-oxides.

The present invention also relates to useful forms of the compounds as disclosed herein, such as metabolites, hydrates, solvates, prodrugs, salts, in particular pharmaceutically acceptable salts, and co-precipitates.

The compounds of the present invention can exist as a hydrate, or as a solvate, wherein the compounds of the present invention contain polar solvents, in particular water, methanol or ethanol for example as structural element of the crystal lattice of the compounds. The amount of polar solvents, in particular water, may exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, the compounds of the present invention can exist in the form of a salt. Said salt may be any salt, either an organic or inorganic addition salt, particularly any pharmaceutically acceptable organic or inorganic addition salt, customarily used in pharmacy.

The term “pharmaceutically acceptable salt” refers to a relatively non-toxic, inorganic or organic acid addition salt of a compound of the present invention. For example, see S. M. Berge, et al. “Pharmaceutical Salts,” J. Pharm. Sci. 1977, 66, 1-19. The production of especially neutral salts is described in U.S. Pat. No. 5,560,903.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, such as hydrochloric, hydrobromic, hydroiodic, sulfuric, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, persulfuric, 3-phenylpropionic, picric, pivalic, 2-hydroxyethanesulfonate, itaconic, sulfamic, trifluoromethanesulfonic, dodecylsulfuric, ethansulfonic, benzenesulfonic, para-toluenesulfonic, methansulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, hemisulfuric, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium or magnesium salt, an ammonium salt or a salt with an organic base which affords a physiologically acceptable cation, for example a salt with N-methyl-glucamine, dimethyl-glucamine, ethyl-glucamine, lysine, dicyclohexylamine, 1,6-hexadiamine, ethanolamine, glucosamine, sarcosine, serinol, tris-hydroxy-methyl-aminomethane, aminopropandiol, sovak-base, 1-amino-2,3,4-butantriol. Additionally, basic nitrogen containing groups may be quaternised with such agents as lower alkyl halides such as methyl, ethyl, propyl, and butyl chlorides, bromides and iodides; dialkyl sulfates like dimethyl, diethyl, and dibutyl sulfate; and diamyl sulfates, long chain halides such as decyl, lauryl, myristyl and strearyl chlorides, bromides and iodides, aralkyl halides like benzyl and phenethyl bromides and others.

Those skilled in the art will further recognise that acid addition salts of the claimed compounds may be prepared by reaction of the compounds with the appropriate inorganic or organic acid via any of a number of known methods. Alternatively, alkali and alkaline earth metal salts of acidic compounds of the invention are prepared by reacting the compounds of the invention with the appropriate base via a variety of known methods.

The present invention includes all possible salts of the compounds of the present invention as single salts, or as any mixture of said salts, in any ratio.

The term “thrombus (thrombi)” describes all kinds of blood clots (venous and arterial thrombi). The term “thrombus (thrombi)” includes also any terms of phrases like “thrombotic deposits” and “thrombus formation sites”. Thrombi usually arise as a result of the blood coagulation step in hemostasis or pathologically as the result of different causes like thrombotic disorders. In this investigation all platelet containing thrombi are included as well as circulating thrombi (embolus), which get stuck somewhere in the vascular tree.

In a second aspect, the present invention covers compounds of general formula (I), supra, in which:

X represents a group selected from:

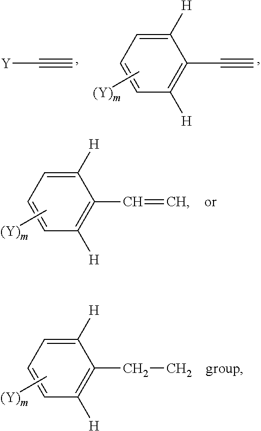

in which groups:

Y represents a:

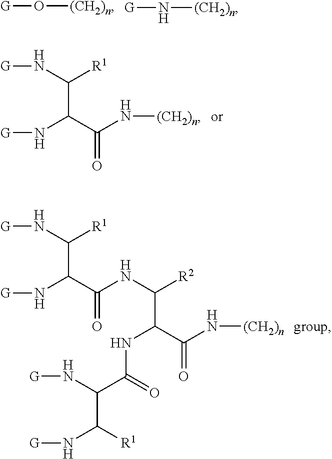

in which groups:

R¹ represents Hydrogen, Methyl, Ethyl, Propyl or iso-Propyl;

R² represents Hydrogen, Methyl, Ethyl, Propyl or iso-Propyl;

G represents a:

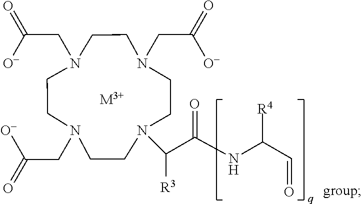

in which:

R³ represents Hydrogen, Methyl, Ethyl, Propyl, iso-Propyl or Benzyl;

R⁴ represents Hydrogen, Methyl, Ethyl, Propyl, iso-Propyl or Benzyl;

M represents Gadolinium;

m represents 1 or 2;

n represents an integer of 2, 3, 4, 5 or 6;

q represents 0 or 1;

or a stereoisomer, a tautomer, an N-oxide, a hydrate, a solvate, or a salt thereof, or a mixture of same.

In a further aspect, the present invention covers compounds of general formula (I), supra, in which:

X represents a group selected from:

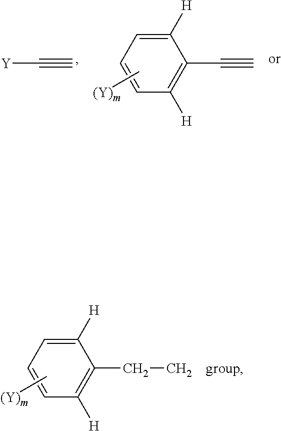

in which groups:

Y represents a:

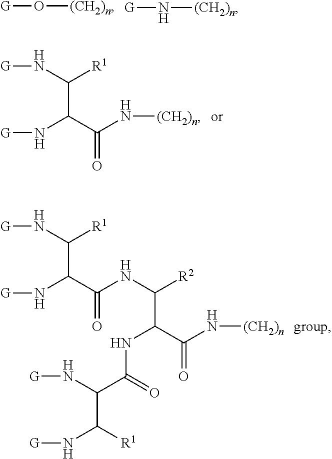

in which groups:

R¹ represents Hydrogen or Methyl;

R² represents Hydrogen or Methyl;

G represents a:

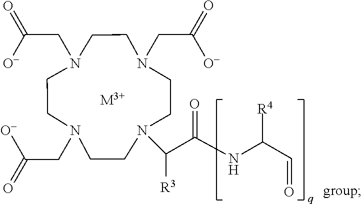

in which:

R³ represents Hydrogen or Methyl;

R⁴ represents Hydrogen or Methyl;

M represents Gadolinium;

m represents 1 or 2;

n represents an integer of 2, 3, 4, 5 or 6;

q represents 0 or 1;

or a stereoisomer, a tautomer, an N-oxide, a hydrate, a solvate, or a salt thereof, or a mixture of same.

In a further aspect, the present invention covers compounds of general formula (I), supra, in which:

X represents a group selected from:

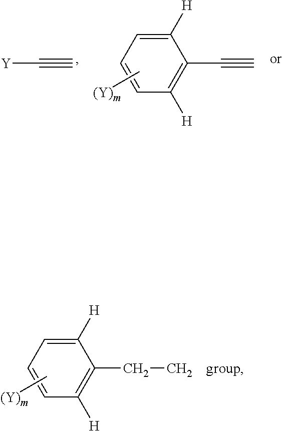

in which groups:

Y represents a:

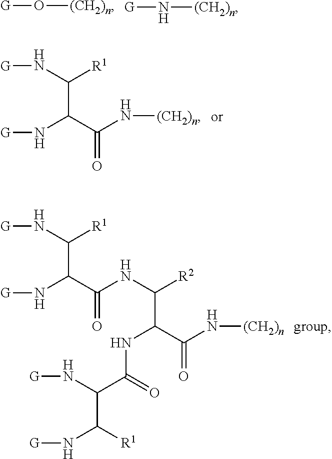

in which groups:

R¹ represents Hydrogen;

R² represents Hydrogen;

G represents a:

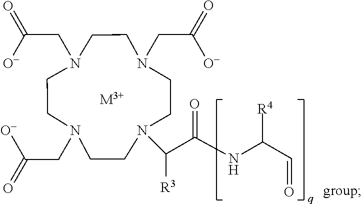

in which:

R³ represents Methyl;

R⁴ represents Hydrogen;

M represents Gadolinium;

m represents 1 or 2;

n represents an integer of 2, 3, 4, 5 or 6;

q represents 1;

or a stereoisomer, a tautomer, an N-oxide, a hydrate, a solvate, or a salt thereof, or a mixture of same.

In a further aspect, the present invention covers compounds of general formula (I), supra, in which

X represents a group selected from:

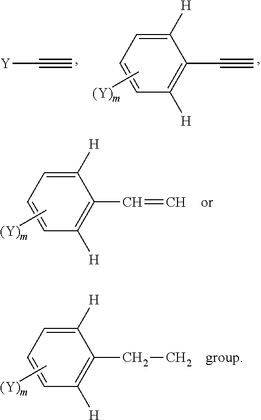

In a further aspect, the present invention covers compounds of general formula (I), supra, in which

X represents a group selected from:

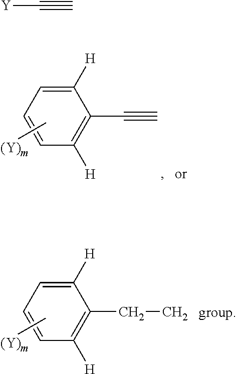

In a further aspect, the present invention covers compounds of general formula (I), supra, in which:

X represents a

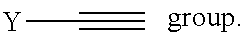

In a further aspect, the present invention covers compounds of general formula (I), supra, in which:

X represents a

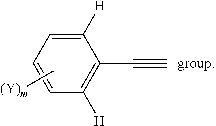

In a further aspect, the present invention covers compounds of general formula (I), supra, in which

X represents a

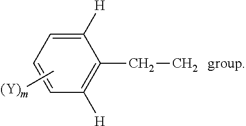

In a further aspect, the present invention covers compounds of general formula (I), supra, in which

Y represents a:

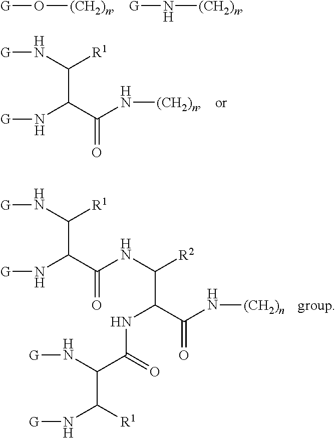

In a further aspect, the present invention covers compounds of general formula (I), supra, in which

Y represents a:

G-O—(CH₂)ₙ group.

In a further aspect, the present invention covers compounds of general formula (I), supra, in which

Y represents a:

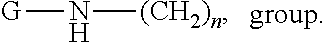

In a further aspect, the present invention covers compounds of general formula (I), supra, in which

Y represents a:

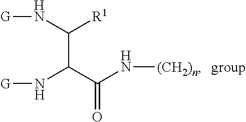

In a further aspect, the present invention covers compounds of general formula (I), supra, in which

Y represents a:

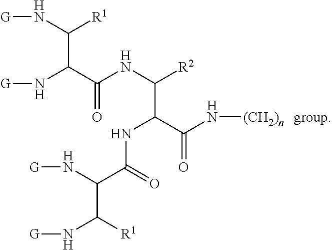

In a further aspect, the present invention covers compounds of general formula (I), supra, in which

R¹ represents Hydrogen, Methyl, Ethyl, Propyl or iso-Propyl.

In a further aspect, the present invention covers compounds of general formula (I), supra, in which

R¹ represents Hydrogen or Methyl.

In a further aspect, the present invention covers compounds of general formula (I), supra, in which

R¹ represents Hydrogen.

In a further aspect, the present invention covers compounds of general formula (I), supra, in which

R¹ represents Methyl.

In a further aspect, the present invention covers compounds of general formula (I), supra, in which

R² represents Hydrogen, Methyl, Ethyl, Propyl or iso-Propyl.

In a further aspect, the present invention covers compounds of general formula (I), supra, in which

R² represents Hydrogen or Methyl.

In a further aspect, the present invention covers compounds of general formula (I), supra, in which

R² represents Hydrogen.

In a further aspect, the present invention covers compounds of general formula (I), supra, in which

R² represents Methyl.

In a further aspect, the present invention covers compounds of general formula (I), supra, in which

R³ represents Hydrogen, Methyl, Ethyl, Propyl, iso-Propyl or Benzyl.

In a further aspect, the present invention covers compounds of general formula (I), supra, in which

R³ represents Hydrogen or Methyl.

In a further aspect, the present invention covers compounds of general formula (I), supra, in which

R³ represents Hydrogen.

In a further aspect, the present invention covers compounds of general formula (I), supra, in which

R³ represents Methyl.

In a further aspect, the present invention covers compounds of general formula (I), supra, in which

R⁴ represents Hydrogen, Methyl, Ethyl, Propyl, iso-Propyl or Benzyl.

In a further aspect, the present invention covers compounds of general formula (I), supra, in which

R⁴ represents Hydrogen or Methyl.

In a further aspect, the present invention covers compounds of general formula (I), supra, in which

R⁴ represents Hydrogen.

In a further aspect, the present invention covers compounds of general formula (I), supra, in which

R⁴ represents Methyl.

In a further aspect, the present invention covers compounds of general formula (I), supra, in which

M represents Praseodymium, Neodymium, Samarium, Ytterbium, Gadolinium, Terbium, Dysprosium, Holmium or Erbium.

In a further aspect, the present invention covers compounds of general formula (I), supra, in which

M represents Gadolinium.

In a further aspect, the present invention covers compounds of general formula (I), supra, in which

m represents 1 or 2.

In a further aspect, the present invention covers compounds of general formula (I), supra, in which

m represents 1.

In a further aspect, the present invention covers compounds of general formula (I), supra, in which

m represents 2.

In a further aspect, the present invention covers compounds of general formula (I), supra, in which

n represents an integer of 2, 3, 4, 5 or 6.

In a further aspect, the present invention covers compounds of general formula (I), supra, in which

n represents an integer of 2.

In a further aspect, the present invention covers compounds of general formula (I), supra, in which

n represents an integer of 3.

In a further aspect, the present invention covers compounds of general formula (I), supra, in which

n represents an integer of 4.

In a further aspect, the present invention covers compounds of general formula (I), supra, in which

n represents an integer of 5.

In a further aspect, the present invention covers compounds of general formula (I), supra, in which

n represents an integer of 6.

In a further aspect, the present invention covers compounds of general formula (I), supra, in which

q represents 0 or 1.

In a further aspect, the present invention covers compounds of general formula (I), supra, in which

q represents 0.

In a further aspect, the present invention covers compounds of general formula (I), supra, in which

q represents 1.

In a further aspect, the present invention covers compounds of general formula (I), selected from the group consisting of:

Gadolinium 2,2′,2″-(10-{(2S)-1-[(2-{[4-(5-{(1S)-2-carboxy-1-[({(3R)-1-[3-(piperidin-4-yl)-propanoyl]piperidin-3-yl}carbonyl)amino]ethyl}pyridin-3-yl)but-3-yn-1-yl]oxy}-2-oxoethyl)-amino]-1-oxopropan-2-yl}-1,4,7,10-tetraazacyclododecane-1,4,7-triyl)triacetate;

Gadolinium 2,2′,2″-(10-{( 2S)-1-[(2-{[4-(5-{(1S)-2-carboxy-1-[({(3R)-1-[3-(piperidin-4-yl)-propanoyl]piperidin-3-yl}carbonyl)amino]ethyl}pyridin-3-yl)but-3-yn-1-yl]amino}-2-oxoethyl)-amino]-1-oxopropan-2-yl}-1,4,7,10-tetraazacyclododecane-1,4,7-triyl)triacetate;

Gadolinium 2,2′,2″-{10-[(2S)-1-({2-[(6-{4-[(5-{(1S)-2-carboxy-1-[({(3R)-1-[3-(piperidin-4-yl)- propanoyl]piperidin-3-yl}carbonyl)amino]ethyl}pyridin-3-yl)ethynyl]phenyl}hexyl)amino]-2-oxoethyl}amino)-1-oxopropan-2-yl]-1,4,7,10-tetraazacyclododecane-1,4,7-triyl}triacetate;

Gadolinium 2,2′,2″-{10-[(2S)-1-({2-[(6-{4-[(5-{(1S)-2-carboxy-1-[({(3R)-1-[3-(piperidin-4-yl)- propanoyl]piperidin-3-yl}carbonyl)amino]ethyl}pyridin-3-yl)ethyl]phenyl}hexyl)amino]-2-oxo-ethyl}amino)-1-oxopropan-2-yl]-1,4,7,10-tetraazacyclododecane-1,4,7-triyl}triacetate;

Gadolinium 2,2′,2″-{10-[(2S)-1-({2-[(6-{3-[(5-{(1S)-2-carboxy-1-[({(3R)-1-[3-(piperidin-4-yl)- propanoyl]piperidin-3-yl}carbonyl)amino]ethyl}pyridin-3-yl)ethynyl]phenyl}butyl)amino]-2-oxo-ethyl}amino)-1-oxopropan-2-yl]-1,4,7,10-tetraazacyclododecane-1,4,7-triyl}triacetate;

Digadolinium 2,2′,2″,2′″,2″″,2′″″-({5-[(5-{(1S)-2-carboxy-1-[({(3R)-1-[3-(piperidin-4-yl)-propanoyl]piperidin-3-yl}carbonyl)amino]ethyl}pyridin-3-yl)ethynyl]-1,3-phenylene}bis[butane-4,1-diylimino(2-oxoethane-2,1-diyl)imino(1-oxopropane-1,2-diyl)-1,4,7,10-tetraazacyclo-dodecane-10,1,4,7-tetrayl])hexaacetate;

Tetragadolinium 2,2′,2″,2′″,2″″,2′″″,2″″″,2′″″″,2″″″″,2′″″″″,2″″″″″,2′″″″″″-({5-[(5-{(1S)-2-carboxy-1-[({(3R)-1-[3-(piperidin-4-yl)propanoyl]piperidin-3-yl}carbonyl)amino]ethyl}pyridin-3-yl)-ethynyl]-1,3-phenylene}bis[butane-4,1-diylcarbamoyl(3,6,11,14-tetraoxo-4,7,10,13-tetraaza-hexadecane-8,2,15-triyl)di-1,4,7,10-tetraazacyclododecane-10,1,4,7-tetrayl])dodecaacetate;

Tetragadolinium N-{2-[4,7,10-tris(carboxylatomethyl)-1,4,7,10-tetraazacyclododecan-1-yl]-propanoyl}glycyl-3-[(N-{2-[4,7,10-tris(carboxylatomethyl)-1,4,7,10-tetraazacyclododecan-1-yl]propanoyl}glycyl)amino]alanyl-N-(4-{3-[(5-{(1S)-2-carboxy-1-[({(3R)-1-[3-(piperidin-4-yl)-propanoyl]piperidin-3-yl}carbonyl)amino]ethyl}pyridin-3-yl)ethynyl]phenyl}butyl)-3-[(N-{2-[4,7,10-tris(carboxylatomethyl)-1,4,7,10-tetraazacyclododecan-1-yl]propanoyl}glycyl-3-[(N-{2-[4,7,10-tris(carboxylatomethyl)-1,4,7,10-tetraazacyclododecan-1-yl]propanoyl}glycyl)amino]-alanyl)amino]alaninamide;

Tetragadolinium 2,3-bis({2,3-bis[(N-{2-[4,7,10-tris(carboxylatomethyl)-1,4,7,10-tetraazacyclododecan-1-yl]propanoyl}glycyl)amino]propanoyl}amino)-N-(4-{3-[(5-{(1S)-2-carboxy-1-[({(3R)-1-[3-(piperidin-4-yl)propanoyl]piperidin-3-yl}carbonyl)amino]ethyl}pyridin-3-yl)ethyl]phenyl}butyl)propanamide;

Digadolinium N-{2-[4,7,10-tris(carboxylatomethyl)-1,4,7,10-tetraazacyclododecan-1-yl]propanoyl}glycyl-N-(3-{4-[(5-{(1S)-2-carboxy-1-[({(3R)-1-[3-(piperidin-4-yl)propanoyl]piperidin-3-yl}carbonyl)amino]ethyl}pyridin-3-yl)ethynyl]phenyl}propyl)-3-[(N-{2-[4,7,10-tris(carboxylatomethyl)-1,4,7,10-tetraazacyclododecan-1-yl]propanoyl}glycyl)amino]alaninamide; and

Tetragadolinium 2,3-bis({2,3-bis[(N-{2-[4,7,10-tris(carboxylatomethyl)-1,4,7,10-tetraazacyclododecan-1-yl]propanoyl}glycyl)amino]propanoyl}amino)-N-(3-{4-[(5-{(1S)-2-carboxy-1-[({1(3R)-1-[3-(piperidin-4-yl)propanoyl]piperidin-3-yl}carbonyl)amino]ethyl}pyridin-3-yl)ethynyl]phenyl}propyl)propanamide.

Another aspect of the invention is the use of a compound of general formula (I) for diagnostic imaging.

Preferably, the use of a compound of the invention in the diagnosis is performed using magnetic resonance imaging (MRI).

The invention also contains compounds of general formula (I) for the manufacture of diagnostic agents.

Another aspect of the invention is the use of the compounds of general formula (I) or mixtures thereof for the manufacture of diagnostic agents.

Another aspect of the invention is the use of the compounds of general formula (I) or mixtures thereof for the manufacture of diagnostic agents for imaging thrombi.

A method of imaging body tissue in a patient, comprising the steps of administering to the patient an effective amount of one or more compounds of general formula (I) in a pharmeutically acceptable carrier, and subjecting the patient to NMR tomography. Such a method is described in U.S. Pat. No. 5,560,903.

For the manufacture of diagnostic agents, for example the adminstration to human or animal subjects, the compounds of general formula (I) or mixtures will conveniently be formulated together with pharmaceutical carriers or excipient. The contrast media of the invention may conveniently contain pharmaceutical formulation aids, for example stabilizers, antioxidants, pH adjusting agents, flavors, and the like. Production of the diagnostic media according to the invention is also performed in a way known in the art, see U.S. Pat. No. 5,560,903. They may be formulated for parenteral or enteral administration or for direct administration into body cavities. For example, parenteral formulations contain a steril solution or suspension in a dosis of 0.0001-5 mmol metal/kg body weight, especially 0.005-0.5 mmol metal/kg body weight of the compound of formula (I) according to this invention. Thus the media of the invention may be in conventional pharmaceutical formulations such as solutions, suspensions, dispersions, syrups, etc. in physiologically acceptable carrier media, preferably in water for injections. When the contrast medium is formulated for parenteral administration, it will be preferably isotonic or hypertonic and close to pH 7.4.

In a further aspect, the invention is directed to a method of diagnosing a patient with a thromboembolic disease, such as myocardial infarction, pulmonary embolism, stroke and transient ischemic attacks. This method comprises a) administering to a human in need of such diagnosis a compound of the invention for detecting the compound in the human as described above and herein, and b) measuring the signal arising from the administration of the compound to the human, preferably by magnetic resonance imaging (MRI).

In a further aspect, the invention is directed to a method of diagnosing a patient with a life threatening disease, such as aortic aneurism, chronic thromboembolic pulmonary hypertension (CETPH), arterial fibrillation and coronary thrombosis. This method comprises a) administering to a human in need of such diagnosis a compound of the invention for detecting the compound in the human as described above and herein, and b) measuring the signal from arising from the administration of the compound to the human, preferably by magnetic resonance imaging (MRI).

In a further aspect, the invention is directed to a method of diagnosing and health monitoring of cardiovascular risk patients. This method comprises a) administering to a human in need of such diagnosis a compound of the invention for detecting the compound in the human as described above and herein, and b) measuring the signal arising from the administration of the compound to the human, preferably by magnetic resonance imaging (MRI).

General Synthesis

The compounds according to the invention can be prepared according to the following schemes 1 through 7.

The schemes and procedures described below illustrate synthetic routes to the compounds of general formula (I) of the invention and are not intended to be limiting. It is obvious to the person skilled in the art that the order of transformations as exemplified in the Schemes can be modified in various ways. The order of transformations exemplified in the Schemes is therefore not intended to be limiting. In addition, interconversion of any of the substituents, R¹, R², R³ and R⁴ can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3rd edition, Wiley 1999). Specific examples are described in the subsequent paragraphs.

The term “amine-protecting group” as employed herein by itself or as part of another group is known or obvious to someone skilled in the art, which is chosen from but not limited to a class of protecting groups namely carbamates, amides, imides, N-alkyl amines, N-aryl amines, imines, enamines, boranes, N—P protecting groups, N-sulfenyl, N-sulfonyl and N-silyl, and which is chosen from but not limited to those described in the textbook Greene and Wuts, Protecting groups in Organic Synthesis, third edition, page 494-653, included herewith by reference. The “amine-protecting group” is preferably carbobenzyloxy (Cbz), p-methoxybenzyl carbonyl (Moz or MeOZ), tert-butyloxycarbonyl (BOC), 9-fluorenylmethyloxycarbonyl (FMOC), benzyl (Bn), p-methoxybenzyl (PMB), 3,4-dimethoxybenzyl (DMPM), p-methoxyphenyl (PMP), triphenylmethyl (Trityl), methoxyphenyl diphenylmethyl (MMT) or the protected amino group is a 1,3-dioxo-1,3-dihydro-2H-isoindol-2-yl (phthalimido) or an azido group.

The term “carboxyl-protecting group” as employed herein by itself or as part of another group is known or obvious to someone skilled in the art, which is chosen from but not limited to a class of protecting groups namely esters, amides and hydrazides, and which is chosen from but not limited to those described in the textbook Greene and Wuts, Protecting groups in Organic Synthesis, third edition, page 369-453, included herewith by reference. The “carboxyl-protecting group” is preferably methyl, ethyl, propyl, butyl, tert-butyl, allyl, benzyl, 4-methoxybenzyl or 4-methoxyphenyl.

In general the synthesis of the GP IIbIIIa binder moiety is documented in the literature:

1) J. Med. Chem. 1999, 42, 5254-5265

2) Organic Progress Research & Development 2003, 7, 866-872

Modifications and improvements of these methods are described in detail in the experimental part. The principal path to the pyridinium bromide A is exemplified in Scheme 1:

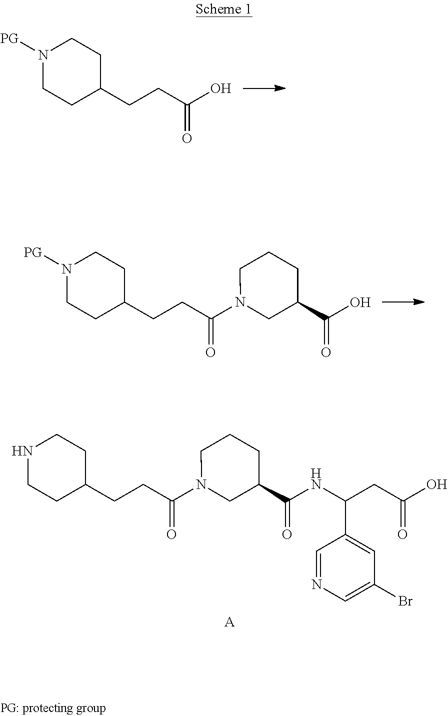

The pyridinium bromide A obtained in the synthesis outlined in Scheme 1 is a mixture of two diastereomers. In general, stereoselective methods for the synthesis of 8-amino acids are applicable (M. Liu, M. P. Sibi, Tetrahedron 2002 58, 7991-8035 or E. Juaristi, V. Soloshonok Eds. of Enantioselective Synthesis of Beta-Amino Acids, second edition, Wiley-Interscience, ISBN 0-471-46738-3).

In a stereoselective approach, which is depicted in Scheme 2, the 3-pyridyl nitrile B can be transformed to the aryl enamine C which is stereoselectively reduced (Yi Hsiao et. al. J. Am. Chem. Soc. 2004 126, 9918-9919) to the enantiomerically enriched 3-amino-3-arylpropanoic acid tert.-butyl ester D. This ester is coupled to the piperidine fragment E via an activated ester to deliver F. Standard protective group transformation delivers the free amino acid. Palladium catalyzed Sonogashira reaction of the bromide with an alkyne connected to the metal complex delivers the compounds of the general formula (I). Preferrably the final coupling reaction is perfomed in a partially aqueous solvent under use of water solouble palladium complexes like {palladium[2-(dimethylaminomethyl)phenyl][1,3,5-triaza-7-phosphaadamantane]chloride (Organometallics 2006, 25, 5768-5773) or trisodium 3,3′,3″-phosphanetriyltris(4,6-dimethylbenzenesulfonate) as palladium ligand (Eur. J. Org. Chem. 2010, 3678-3683).

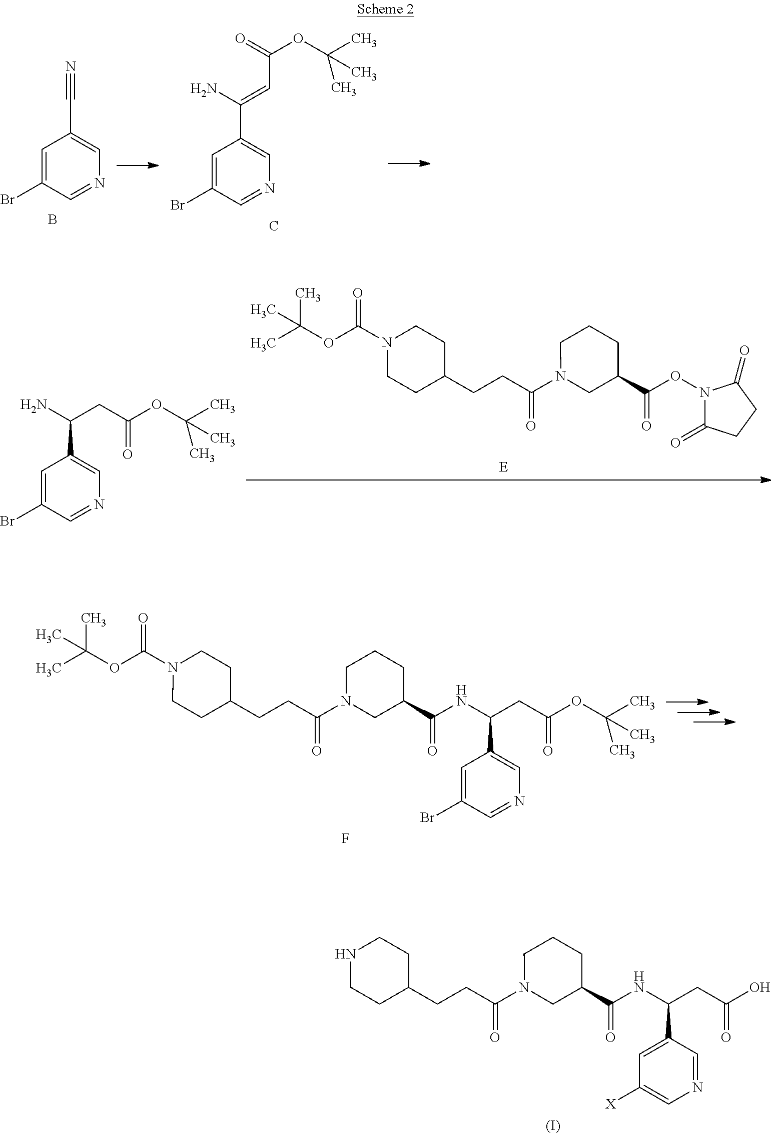

Isolation and purification of the desired metal complex conjugates of the general formula (I) can be achieved by conventional chromatographic methods like preparative HPLC or size exclusion chromatography in case of poly Gadolinium complexes in combination with ultrafiltration methods.

The synthesis of compounds of the general formula (Ia) is depicted in scheme 3.

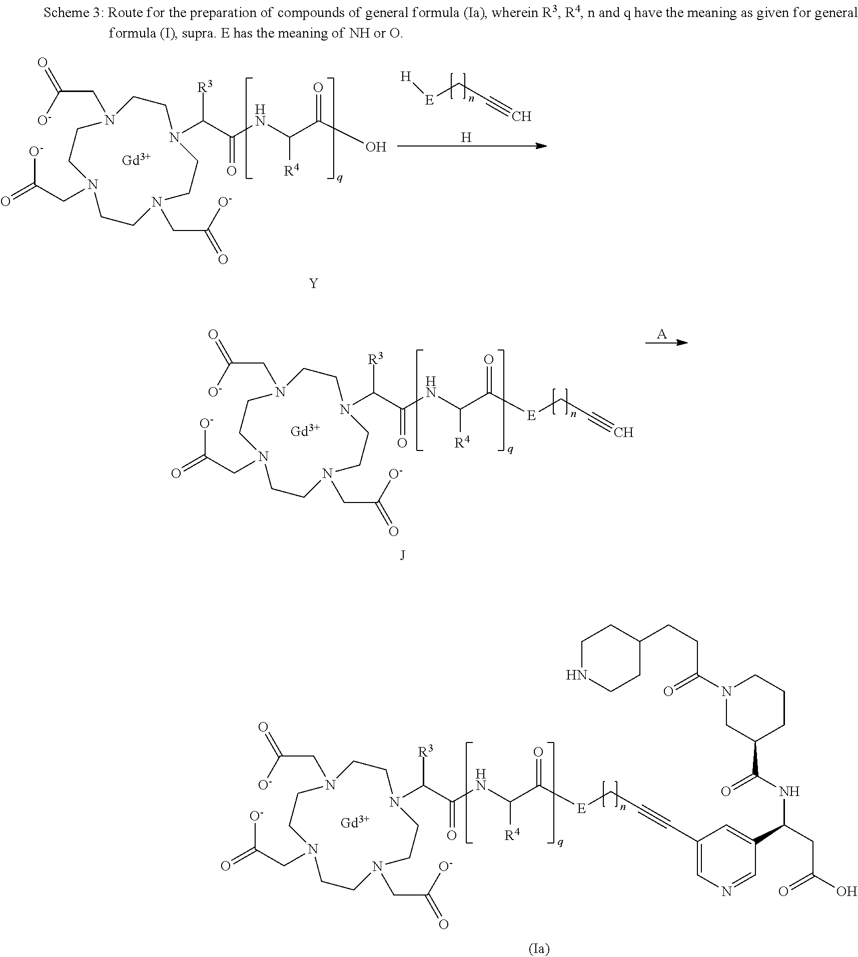

Alkynes of general formula H are either commercially available, or are described in the literature, or can be prepared from known starting materials, employing standard reactions which are well known to the person skilled in the art.

Gadolinium complexes of general formula Y can be converted to compounds of general formula J by reaction with an alkyne of general formula H.

Compounds of general formula J, wherein E has the meaning of O can be obtained by reaction of the respective acetylenic alcohol H, using, for example, coupling reagents such as diisopropyl azadicarboxylate in the presence of triphenylphosphine, in a solvent such as for example, DMF, in a temperature range from −30° C. to 60° C., preferably the reaction is carried out at 0° C.

Compounds of general formula J, wherein E has the meaning of NH can be obtained in an analoguous manner by reaction of the respective acetylenic amine H, using, for example, coupling reagents such as HATU, in the presence of a suitable base, such as for example, N-ethyldiisopropyl amine, in solvents, such as for example DMF or DMSO or mixtures thereof, in a temperature range from −30° C. to 80° C., preferably the reaction is carried out at 20° C.

Compounds of general formula J can be converted to compounds of general formula (Ia) by a Palladium catalyzed Sonogashira reaction with the bromide A, employing a suitable palladium catalyst, such as for example tetrakis(triphenylphoshine)palladium(0), and copper(I)iodide, in the presence of a suitable base, such as for example piperidine, using a solvent as for example DMF, or using a water solouble palladium complex as {palladium[2-(dimethylaminomethyl)phenyl][1,3,5-triaza-7-phosphaadamantane]chloride (Organometallics 2006, 25, 5768-5773) or trisodium 3,3′,3″-phosphanetriyltris(4,6-dimethylbenzenesulfonate) (Eur. J. Org. Chem. 2010, 3678-3683), in a partially aqueous solvent, such as for example a mixture of acetonitrile and water, in a temperature range from room temperature to the boiling point of the respective solvent, preferably the reaction is carried out in a temperature range from 80° C. to 100° C.

The synthesis of compounds of the general formulae (Ib), (Ic) and (Id) is depicted in scheme 4.

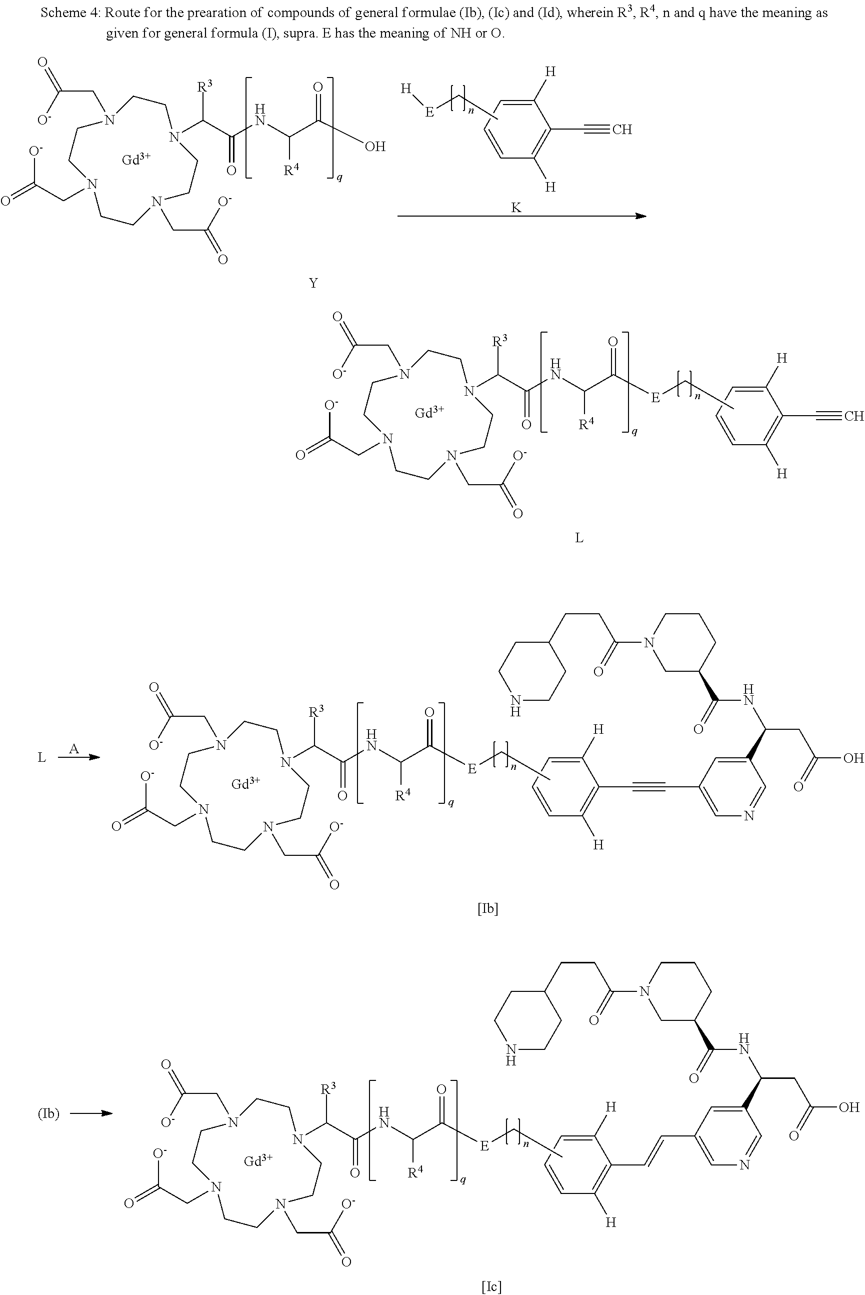

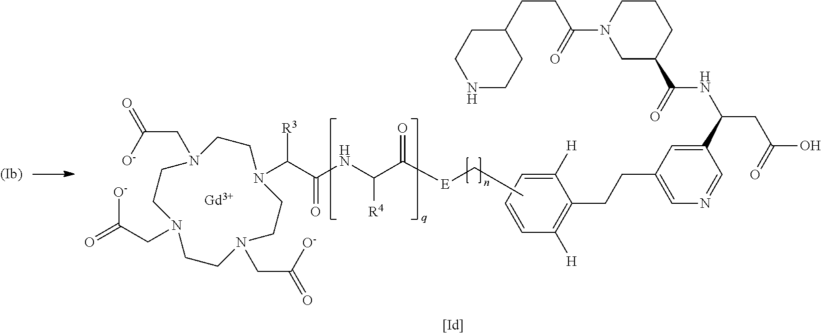

Alkynes of general formula K are either commercially available, or are described in the literature, or can be prepared from known starting materials, employing standard reactions which are well known to the person skilled in the art.

Gadolinium complexes of general formula Y can be converted to compounds of general formula L by reaction with a phenylacetylene derivative of general formula K, employing suitable coupling methods, as described, for example, for the analogous synthesis depicted in scheme 3.

Compounds of general formula L can be converted to compounds of general formula (Ib) by a Palladium catalyzed Sonogashira reaction with the bromide A, employing a suitable palladium catalyst, such as for example tetrakis(triphenylphoshine)palladium(0), and copper(I)iodide, in the presence of a suitable base, such as for example piperidine, using a solvent, such as for example DMF, or using a water solouble palladium complex as {palladium[2-(dimethylaminomethyl)phenyl][1,3,5-triaza-7-phosphaadamantane]chloride (Organometallics 2006, 25, 5768-5773) or trisodium 3,3′,3″-phosphanetriyltris(4,6-dimethylbenzenesulfonate) (Eur. J. Org. Chem. 2010, 3678-3683), in a partially aqueous solvent, such as for example a mixture of acetonitrile and water, in a temperature range from room temperature to the boiling point of the respective solvent, preferably the reaction is carried out in a temperature range from 60° C. to 80° C.

Compounds of general formula (Ib) can be transferred to compounds of general formula (Ic) by partial hydrogenation, or can be transferred to compounds of general formula (Id) by complete hydrogenation, employing hydrogenation catalysts and reaction conditions which are well known to the person skilled in the art.

Compounds of general formula (Ic) can be transferred to compounds of general formula (Id) by hydrogenation, employing hydrogenation catalysts and reaction conditions which are well known to the person skilled in the art.

The synthesis of compounds of the general formulae (Ie), (If) and (Ig) is depicted in scheme 5.

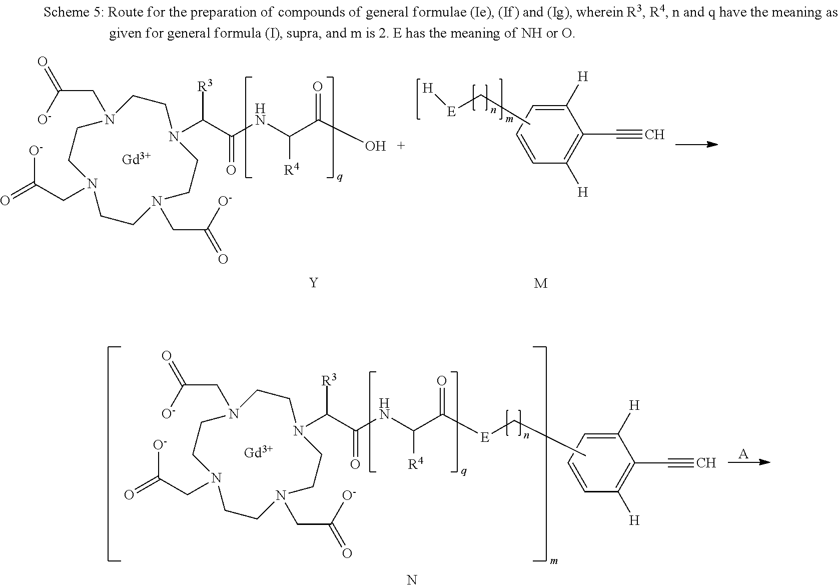

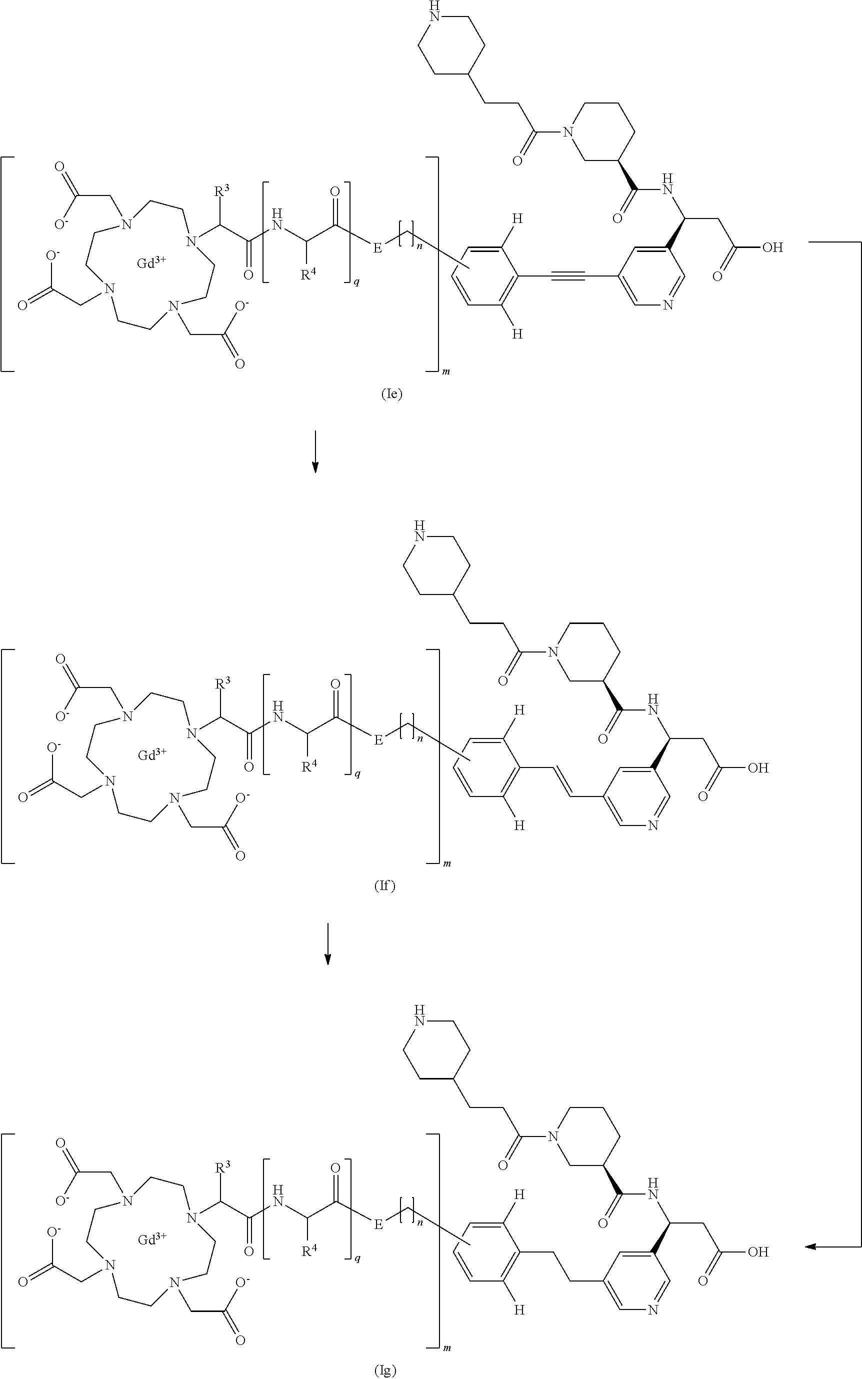

Alkynes of general formula M are either described in the literature, or can be prepared from known starting materials, employing standard reactions which are well known to the person skilled in the art.

Gadolinium complexes of general formula Y can be converted to compounds of general formula N by reaction with a phenylacetylene derivative of general formula M, employing suitable coupling methods, as described, for example, for the analogous synthesis depicted in schemes 3 and 4.

Compounds of general formula N can be converted to compounds of general formula (Ie) by a Palladium catalyzed Sonogashira reaction with the bromide A, employing a suitable palladium catalyst, such as for example {2-[(dimethylamino)methyl]phenyl}palladium(I)-chloride-1,3,5-triaza-7-phosphatricyclo[3.3.1.1]decane, and a suitable base, such as for example triethylamine, using a partially aqueous solvent, such as for example a mixture of acetonitrile and water, in a temperature range from room temperature to the boiling point of the respective solvent, preferably the reaction is carried out in a temperature range from 60° C. to 80° C.

Compounds of general formula (Ie) can be transferred to compounds of general formula (If) by partial hydrogenation, or can be transferred to compounds of general formula (Ig) by complete hydrogenation, employing hydrogenation catalysts and reaction conditions which are well known to the person skilled in the art.

Compounds of general formula (If) can be transferred to compounds of general formula (Ig) by hydrogenation, employing hydrogenation catalysts and reaction conditions which are well known to the person skilled in the art.

The synthesis of compounds of the general formulae (Ih), (Ij) and (Ik) is depicted in scheme 6.

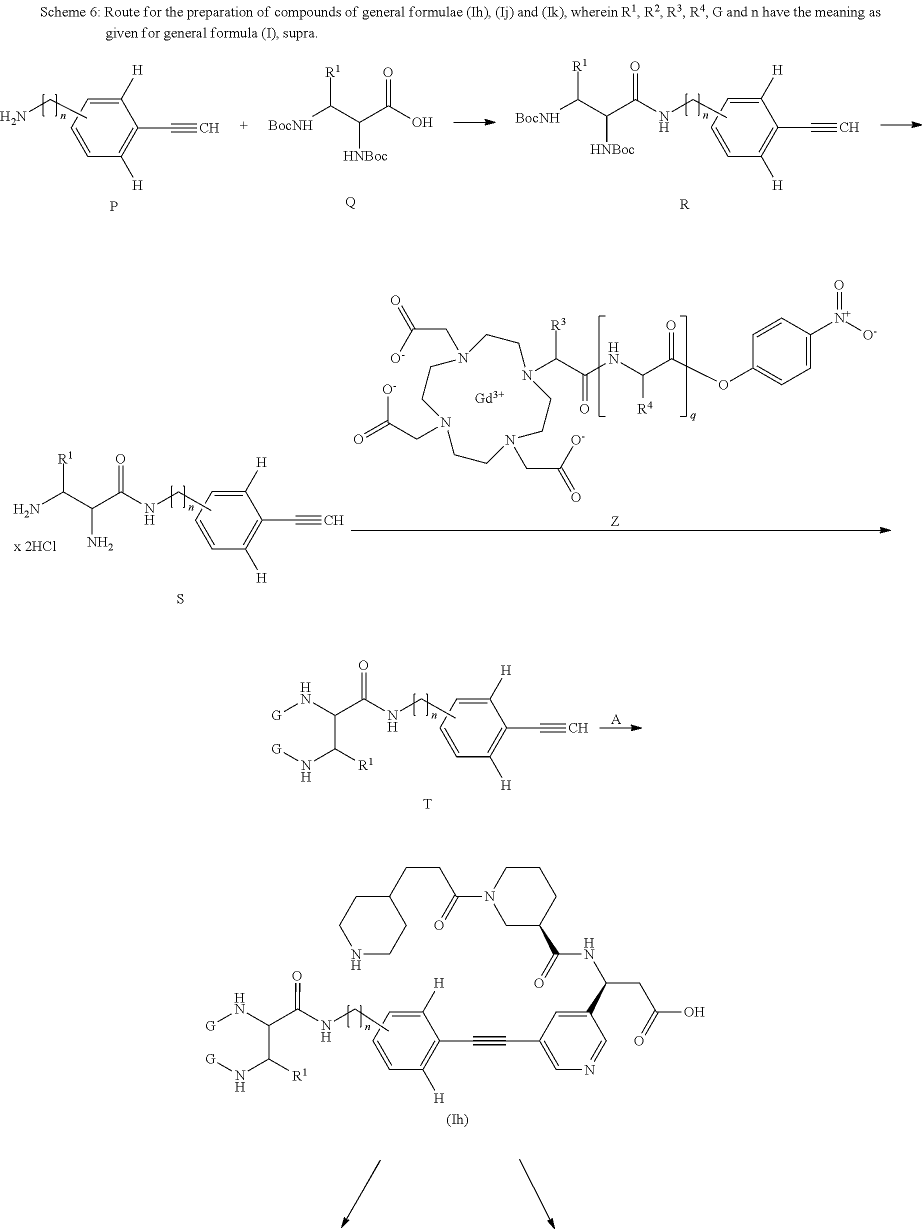

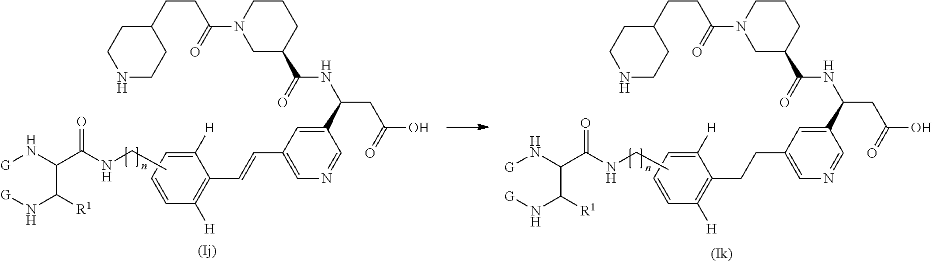

Alkynes of general formula P and Boc protected amino acids of general formula Q are either commercially available, or are described in the literature, or can be prepared from known starting materials, employing standard reactions which are well known to the person skilled in the art. p-Nitrophenyl esters of general formula Z are described in the literature, or can be prepared from known starting materials, employing standard reactions which are well known to the person skilled in the art.

Intermediates of formula P can be converted to protected compounds of general formula R by reaction with a protected amino acid of general formula Q using, for coupling reagents, such as for example HATU, in the presence of a suitable base, such as for example N,N-diisopropylethyl amine, in a solvent, such as for example DMF, in a temperature range from room temperature to the boiling point of the respective solvent, preferably the reaction is carried out at 0° C.

Intermediates of general formula R can be deprotected to compounds of general formula S by standard methods, such as for example by treatment with hydrochloric acid, optionally performing the reaction in a microwave oven, in a solvent, such as for example dioxane or DMF or mixtures thereof, in a temperature range from room temperature to the boiling point of the respective solvent, preferably the reaction is carried out at 80° C.

Intermediates of general formula S can be converted to compounds of general formula T by reaction with compounds of general formula Z, employing a suitable base, such as for example triethylamine, in a solvent, such as for example DMSO or pyridine, in a temperature range from 0° C. to the boiling point of the respective solvent, preferably the reaction is carried out in a temperature range from 50° C. to 60° C.

Alternatively, intermediates of general formula T can be obtained by the reaction of intermediates of general formula S with intermediates of general formula Y, as described, for example for the analogous synthesis depicted in schemes 3 and 4.

Compounds of general formula T can be converted to compounds of general formula (Ih) by a Palladium catalyzed Sonogashira reaction with the bromide A, employing a suitable palladium catalyst, such as for example {2-[(dimethylamino)methyl]phenyl}palladium(I)-chloride-1,3,5-triaza-7-phosphatricyclo[3.3.1.1]decane, and a suitable base, such as for example triethylamine, using a partially aqueous solvent, such as for example a mixture of acetonitrile and water, in a temperature range from room temperature to the boiling point of the respective solvent, preferably the reaction is carried out in a temperature range from 60° C. to 80° C.

Compounds of general formula (Ih) can be transferred to compounds of general formula (Ij) by partial hydrogenation, or can be transferred to compounds of general formula (Ik) by complete hydrogenation, employing hydrogenation catalysts and reaction conditions which are well known to the person skilled in the art.

Compounds of general formula (Ij) can be transferred to compounds of general formula (Ik) by hydrogenation, employing hydrogenation catalysts and reaction conditions which are well known to the person skilled in the art.

The synthesis of compounds of the general formulae (Im), (In) and (Io) is depicted in scheme 7.

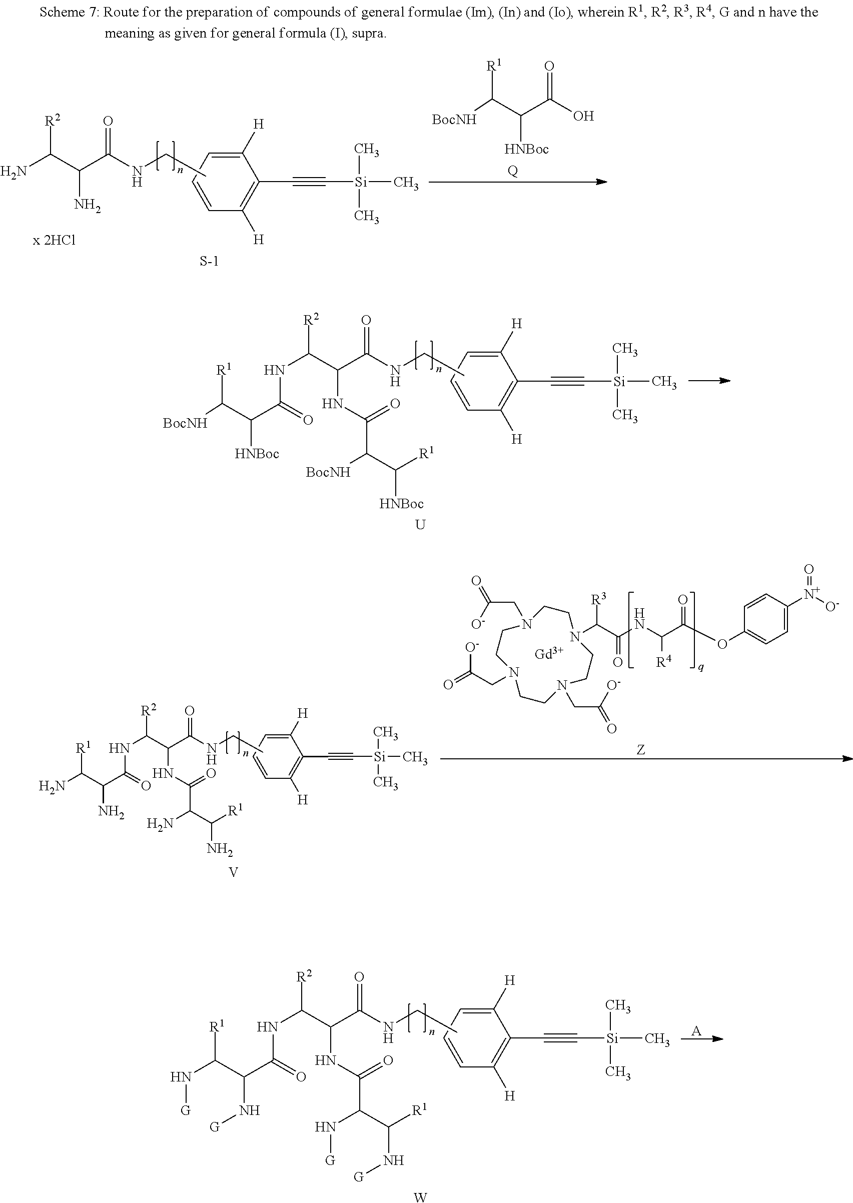

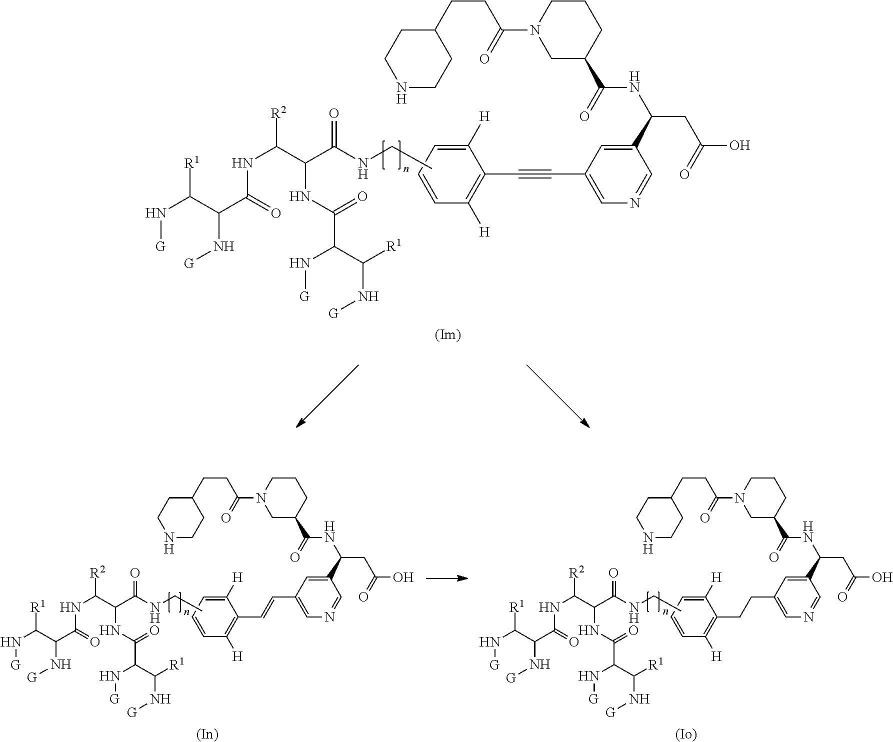

Trimethylsilyl protected alkynes of general formula S-1 can be prepared in analogy to the synthesis of the alkynes of general formula S, which is depicted in scheme 6, from known starting materials, employing standard reactions which are well known to the person skilled in the art.

Intermediates of formula S-1 can be converted to protected compounds of general formula U by reaction with a protected amino acid of general formula Q, using coupling reagents, such as for example HATU, in the presence of a suitable base, such as for example N,N-diisopropylethyl amine, in a solvent, such as for example DMF, in a temperature range from −30° C. to 50° C., preferably the reaction is carried out at 0° C.

Intermediates of general formula U can be deprotected to compounds of general formula V by standard methods, such as for example by treatment with hydrochloric acid, optionally performing the reaction in a microwave oven, in a solvent such as for example dioxane or DMF or mixtures thereof, in a temperature range from room temperature to the boiling point of the respective solvent, preferably the reaction is carried out at 80° C.

Intermediates of general formula V can be converted to compounds of general formula W by reaction with compounds of general formula Z, employing a suitable base, such as for example triethylamine, in a solvent, such as for example DMSO or pyridine, in a temperature range from 0° C. to the boiling point of the respective solvent, preferably the reaction is carried out in a temperature range from 50° C. to 60° C.

Compounds of general formula W can be converted to compounds of general formula (Im) employing a one pot procedure, the first step being the deprotection of the acetylene of compounds of general formula W by reaction with TBAF or tetramethylammonium fluoride, in the presence of a base, such as for example triethylamine, and the second step being a Palladium catalyzed Sonogashira reaction with the bromide A, employing a suitable palladium catalyst, such as for example a catalyst prepared by heating palladium(II)acetate with trisodium 3,3′,3″-phosphanetriyltris(4,6-dimethylbenzenesulfonate)tetrakis (triphenylphoshine)palladium(0). The reaction is carried out in a temperature range from room temperature to the boiling point of the respective solvent, preferably the reaction is carried out in a temperature range from 40° C. to 60° C.

Compounds of general formula (Im) can be transferred to compounds of general formula (In) by partial hydrogenation, or can be transferred to compounds of general formula (Io) by complete hydrogenation, employing hydrogenation catalysts and reaction conditions which are well known to the person skilled in the art.

Compounds of general formula (In) can be transferred to compounds of general formula (Io) by hydrogenation, employing hydrogenation catalysts and reaction conditions which are well known to the person skilled in the art.

### DESCRIPTION OF THE FIGURES

FIG. 1:

Affinity assay: In the first step human GPIIb/IIIa purified from human platelets was immobilized on a 96-well solid plate. After 48 hours the plates were washed and the unspecific binding sites were blocked with Roti®-Block. 2. In the next step, the plates were simultaneously incubated with a tritium labeled known GPIIb/IIIa binder (³H) mixed with increasing concentrations of the novel compounds (inhibitor). The higher the affinity of the inhibitor, the lower the bound fraction of the tritiated known GPIIb/IIIa binder (³H) was. The fraction of tritiated compound (³H), which is not displaced by inhibitor, was measured in a microplate scintillation counter.

FIG. 2:

Magnetic resonance imaging of in vitro platelet-rich thrombi and incubation solution (example 8) using a 3D turbo spin echo sequence (1.5 T, Siemens Avanto, small extremity coil, TR 1050 ms, TE 9.1 ms, 0.5×0.5×0.6 mm³). In FIG. 2a an in vitro control thrombus without the addition of a contrast agent is shown. The signal intensity of the control thrombus is slightly higher than the surrounding medium but clearly lower than the signal of the in vitro thrombus which was incubated with example 8 as depicted in FIG. 2b. In FIG. 2c the incubation solution with a final concentration of 10 μmol substance/L of example 8 in human plasma is represented. The signal intensity is higher than the surrounding plasma solutions in the in vitro platelet-rich thrombi 2a and 2b.

The in vitro thrombus in FIG. 2b is incubated with the solution which is depicted in FIG. 2c. After 20 min incubation period the thrombi was washed three times with plasma solution. The signal intensity of the incubated in vitro thrombus in FIG. 2b shows a clearly higher signal than the control thrombi in FIG. 2a.

### EXPERIMENTAL PART

Abbreviations

| | |
|---|---|
| ACN | acetonitrile |
| Boc | tert-butoxycarbonyl |
| br | broad signal (in NMR data) |
| C_{Gd} | concentration of the compound normalized to the Gadolium |
| CI | chemical ionisation |
| d | doublet |
| DAD | diode array detector |
| dd | doublet of doublet |
| ddd | doublet of doublet of doublet |
| dt | doublet of triplet |
| DMF | N,N-dimethylformamide |
| DMSO | dimethylsulfoxide |
| EI | electron ionisation |
| ELSD | evaporative light scattering detector |
| ESI | electrospray ionisation |
| EtOAc | ethyl acetate |
| EtOH | ethanol |
| Fmoc | fluorenylmethyloxycarbonyl |
| Fu | Fraction unbound |
| GP IIb/IIIa | glycoprotein IIb/IIIa |
| Hal | halogenide |
| HATU | N-[(dimethylamino)(3H-[1,2,3]triazolo[4,5-b]pyridin-3-yloxy)- |
| | methylidene]-N-methylmethanaminium hexafluorophosphate |
| HPLC | high performance liquid chromatography |
| HT | High throughput |
| K₂CO₃ | potassium carbonate |
| MBq | Mega Bequerel |
| LCMS | Liquid chromatography-mass spectroscopy |
| MWCO | Molecular weight cut off |
| MeCN | acetonitrile |
| MeOH | methanol |
| MS | mass spectrometry |
| MTB | methyl tert-butyl ether |
| m | multiplet |
| mc | centred multiplet |
| NH₄Cl | ammonium chloride |
| NMR | nuclear magnetic resonance spectroscopy: chemical shifts (δ) |
| | are given in ppm. |
| q | quadruplett (quartet) |
| quin | quintet |
| rᵢ | (where i = 1, 2) relaxivities in L mmol⁻¹ s⁻¹ |
| Rt | Retention time |
| RT | room temperature |
| s | singlet |
| Rᵢ | (where i = 1, 2) relaxation rates (1/T_{1,2)} |
| Rᵢ₍₀₎ | relaxation rate of the respective solvent |
| T_{1,2} | relaxation time |
| t | triplet |
| TBAF | tetrabutylammonium fluoride |
| TE | time to echo |
| TEE | transesophageal Echocardiography |
| THF | tetrahydrofuran |
| THP | tetrahydropyran |
| TIA | transient ischemic attack |
| TR | time to repetition |
| TSE | turbo spin echo sequence |
| UPLC | ultra performance liquid chromatography |

Materials and Instrumentation

The chemicals used for the synthetic work were of reagent grade quality and were used as obtained.

¹H-NMR spectra were measured in CDCl₃, D₂O or DMSO-d₆, respectively (294 K, Bruker DRX Avance 400 MHz NMR spectrometer (B₀=9.40 T), resonance frequencies: 400.20 MHz for ¹H 300 MHz spectrometer for ¹H. Chemical shifts are given in ppm relative to sodium (trimethylsilyl)propionate-d₄ (D₂O) or tetramethylsilane (DMSO-d₆) as internal standards (δ=0 ppm).

Examples were analyzed and characterized by the following HPLC based analytical methods to determine characteristic retention time and mass spectrum:

Method 1: UPLC (ACN-HCOOH):

Instrument: Waters Acquity UPLC-MS SQD 3001; column: Acquity UPLC BEH C18 1.7 50×2.1 mm; eluent A: water+0.1% formic acid, eluent B: acetonitril; gradient: 0-1.6 min 1-99% B, 1.6-2.0 min 99% B; flow 0.8 ml/min; temperature: 60° C.; injection: 2 μl; DAD scan: 210-400 nm; ELSD

Method 2: UPLC (ACN-HCOOH Polar):

Instrument: Waters Acquity UPLC-MS SQD 3001; column: Acquity UPLC BEH C18 1.7 50×2.1 mm; eluent A: water+0.1% formic acid, eluent B: acetonitril; gradient: 0-1.7 min 1-45% B, 1.7-2.0 min 45-99% B; flow 0.8 ml/min; temperature: 60° C.; injection: 2 μl; DAD scan: 210-400 nm; ELSD

### EXAMPLES

### Example 1

### Gadolinium 2,2′,2″-(10-{(2S)-1-[(2-{[4-(5-{(1S)-2-carboxy-1-[({(3R)-1-[3-(piperidin-4-yl)-propanoyl]piperidin-3-yl}carbonyl)amino]ethyl}pyridin-3-yl)but-3-yn-1-yl]oxy}-2-oxo-ethyl)amino]-1-oxopropan-2-yl}-1,4,7,10-tetraazacyclododecane-1,4,7-triyl)triacetate

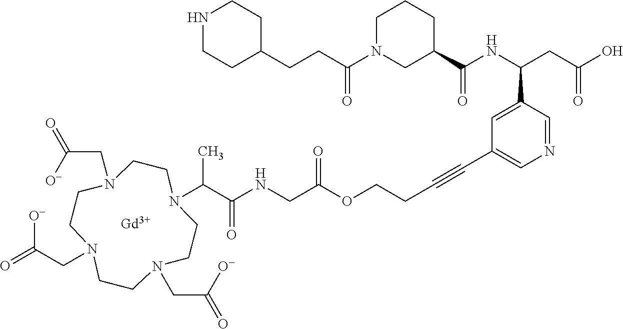

### Example 1a

### Tert-butyl 3-amino-3-[5-bromopyridin-3-yl]prop-2-enoate

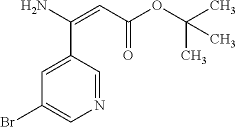 [SMILES: CC(C)(C)OC(=O)/C=C(/N)c1cncc(Br)c1]

Diisopropyl amine (9.2 mL, 65 mmol) was added at 0° C. to a 3M solution of ethyl magnesium bromide in diethyl ether (10.9 mL, 32.7 mmol) and additional diethyl ether (20 mL). After one hour at 0° C. tert-butyl acetate (4.3 mL, 32.7 mmol) was added and stirring was continued for 30 minutes. 5-Bromopyridine-3-carbonitrile (2.0 g, 10.9 mmol) in diethyl ether (42 mL) was added at 0° C. After two hours at 0° C. saturated aqueous ammonium chloride solution was added. Phases were separated and the aqueous phase was extracted with diethyl ether. The combined extracts were washed with brine and dried over sodium sulfate. The solution was concentrated under reduced pressure and the residue was purified by chromatography on silica gel (ethyl acetate in hexane, 0 to 60%) to yield 1.12 g tert-butyl 3-amino-3-(5-bromopyridin-3-yl)prop-2-enoate.

¹H-NMR (400 MHz, DMSO-d₆): δ=1.44 (s, 9H), 4.77 (s, 1H), 7.15 (br., 2H), 8.22 (t, 1H), 8.75 (d, 1H), 8.76 (d, 1H) ppm.

### Example 1b

### Tert-butyl (3S)-3-amino-3-(5-bromopyridin-3-yl)propanoate

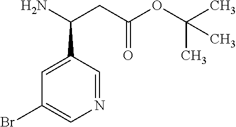 [SMILES: CC(C)(C)OC(=O)C[C@H](N)c1cncc(Br)c1]

To chloro(1,5-cyclooctadien)rhodium(I) dimer (39 mg, 80 μmol) and (R)-(−)-1-[(S)-2-di-tert.-butyl-phosphino)ferrocenyl]ethyldi-(4-trifluormethylphenyl)phosphine (108 mg, 160 μmol) under an argon atmosphere was added 2,2,2-trifluoroethanol (5.8 mL) and the solution was stirred for 40 minutes. To tert-butyl 3-amino-3-(5-bromopyridin-3-yl)prop-2-enoate (1.59 g 5.32 mmol) in degassed 2,2,2-trifluoroethanol (11.6 mL) in a pressure vessel was added the rhodium catalyst solution and the solution was stirred for 22 hours at 50° C. under hydrogen pressure of 11 bar. The solution was concentrated under reduced pressure and the residue was purified by chromatography on silica gel (ethyl acetate in hexane, 12 to 100% followed by methanol in ethyl acetate 0 to 15%) to yield 1.16 g of enantiomerically enriched tert-butyl (3S)-3-amino-3-[5-(benzyloxy)pyridin-3-yl]propanoate.

¹H-NMR (300 MHz, CDCl₃): δ=1.43 (s, 9H), 2.59 (d, 2H), 4.42 (t, 1H), 7.92 (t, 1H), 8.58 (d, 1H), 8.53 (d, 1H) ppm.

α=−17.6° (c=1.0 g /100 mL, CHCl₃).

### Example 1c

### Tert-butyl 4-{3-[(3R)-3-{[(2,5-dioxopyrrolidin-1-yl)oxy]carbonyl}piperidin-1-yl]-3-oxo-propyl}piperidine-1-carboxylate

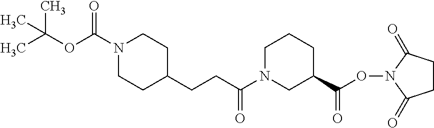 [SMILES: CC(C)(C)OC(=O)N1CCC(CCC(=O)N2CCC[C@@H](C(=O)ON3C(=O)CCC3=O)C2)CC1]

To (3R)-1-{3-[1-(tert-butoxycarbonyl)piperidin-4-yl]propanoyl}piperidine-3-carboxylic acid (1.91 g, 5.18 mmol, Bioorg. Med. Chem. 2005, 13, 4343-4352, Compound 10) in 1,2-dimethoxyethane (13.5 mL) was added N-hydroxysuccinimide (0.60 g, 5.18 mmol) and 1,3-dicyclohexyl carbodiimide (1.18 g, 5.7 mmol). The solution was stirred for 4 hours at room temperature while a precipitate formed. The mixture was then cooled to 0° C. filtrated and the solid washed with diethyl ether. The filtrate and the diethyl ether wash were combined and concentrated to yield 2.61 g of raw tert-butyl 4-{3-[(3R)-3-{[(2,5-dioxopyrrolidin-1-yl)oxy]carbonyl}piperidin-1-yl]-3-oxopropyl}piperidine-1-carboxylate.

UPLC (ACN-HCOOH): Rt.=1.13 min.

MS (ES+): m/e=466.31 (M+H⁺).

### Example 1d

### Tert-butyl 4-{3-[(3R)-3-({(1S)-1-[5-bromopyridin-3-yl]-3-tert-butoxy-3-oxopropyl}carbamoyl)piperidin-1-yl]-3-oxopropyl}piperidine-1-carboxylate

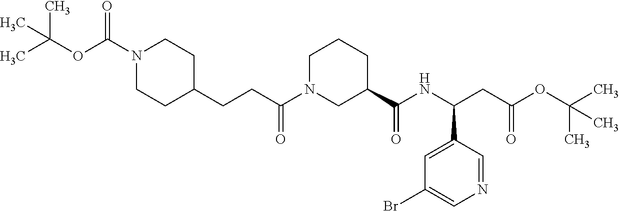 [SMILES: CC(C)(C)OC(=O)C[C@H](NC(=O)[C@@H]1CCCN(C(=O)CCC2CCN(C(=O)OC(C)(C)C)CC2)C1)c1cncc(Br)c1]

To tert-butyl (3S)-3-amino-3-(5-bromopyridin-3-yl)propanoate (1.33 g, 4.42 mmol) in DMF (17 mL) was added tert-butyl 4-{3-[(3R)-3-{[(2,5-dioxopyrrolidin-1-yl)oxy]carbonyl}piperidin-1-yl]-3-oxopropyl}piperidine-1-carboxylate (2.54 g, 4.91 mmol) and triethylamine (1.85 mL, 13.2 mmol) in dichloromethane (17 mL) at 0° C. After 3 hours the mixture was quenched by addition of saturated aqueous ammonium chloride solution, phases were separated and the aqueous phase was extracted with diethyl ether. Combined organic extracts were dried over sodium sulphate, concentrated under reduced pressure and the residue was purified by chromatography on silica gel (ethyl acetate in hexane, 12 to 100% followed by methanol in ethyl acetate 0 to 15%) to yield 2.1 g of tert-butyl 4-[3-((3R)-3-{[(1S)-1-(5-bromopyridin-3-yl)-3-tert-butoxy-3-oxopropyl]carbamoyl}piperidin-1-yl)-3-oxopropyl]piperidine-1-carboxylate.

UPLC (ACN-HCOOH): Rt.=1.35 min.

MS (ES⁺): m/e=651.4/653.4 (M+H⁺).

### Example 1e

### (3S)-3-(5-Bromopyridin-3-yl)-3-[({(3R)-1-[3-(piperidin-4-yl)propanoyl]piperidin-3-yl}-carbonyl)amino]propanoic acid

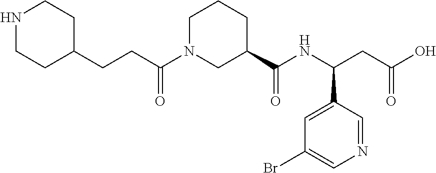 [SMILES: O=C(O)C[C@H](NC(=O)[C@@H]1CCCN(C(=O)CCC2CCNCC2)C1)c1cncc(Br)c1]

Tert-butyl 4-[3-((3R)-3-{[(1S)-1-(5-bromopyridin-3-yl)-3-tert-butoxy-3-oxopropyl]carbamoyl}piperidin-1-yl)-3-oxopropyl]piperidine-1-carboxylate (600 mg, 0.94 mmol) was dissolved in formic acid and heated to 100° C. for 12 minutes. The solvent was destilled off in vacuum and the residue purified by preparative HPLC (C18-Chromatorex-10 μm). To yield 330 mg of (3S)-3-(5-bromopyridin-3-yl)-3-[({(3R)-1-[3-(piperidin-4-yl)propanoyl]piperidin-3-yl}carbonyl)amino]propanoic acid.

UPLC (ACN-HCOOH): Rt.=0.57 min.

MS (ES⁺): m/e=495.2, 497.2 (M+H⁺).

### Example 1f

### Gadolinium 2,2′,2″{10-[1-({2-[(but-3-yn-1-yl)oxy]-2-oxoethyl}amino)-1-oxopropan-2-yl]-1,4,7,10-tetraazacyclododecane-1,4,7-triyl}triacetate

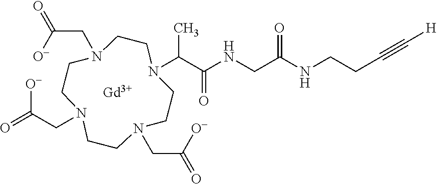

Triphenylphosphine (833 mg, 3.18 mmol) and the gadolinium complex of 10-(4-carboxy-1-methyl-2-oxo-3-azabutyl)-1,4,7,10-tetraazacyclododecane-1,4,7-triacetic acid (Example 1f of EP 0946525, 1.0 g, 1.59 mmol) were solved in DMF (17 mL). 3-Butin-1-ol and diisopropyl azadicarboxylate were added at 0° C. After one day at 0° C. the addition of 3-butin-1-ol and diisopropyl azadicacboxylate was repeated. After 3 hours a mixture of water and ethyl acetate was added, the phases were separated and the organic phase was extracted with water. The aqueous phase was concentrated under reduced pressure and purified by preparative HPLC (C18-YMC ODS AQ-10 μm, acetonitrile in water+0.1% formic acid 1% to 40%) to yield 328 mg of gadolinium 2,2′,2″{10-[1-({2-[(but-3-yn-1-yl)oxy]-2-oxoethyl}amino)-1-oxopropan-2-yl]-1,4,7,10-tetraazacyclododecane-1,4,7-triyl}triacetate.

UPLC (ACN-HCOOH polar): Rt.=0.50 min.

MS (ES⁻): m/e=680.9 (M−H⁺).

### Example 1g

### Gadolinium 2,2′,2″-(10-{(2S)-1-[(2-{[4-(5-{(1S)-2-carboxy-1-[({(3R)-1-[3-(piperidin-4-yl)-propanoyl]piperidin-3-yl}carbonyl)amino]ethyl}pyridin-3-yl)but-3-yn-1-yl]oxy}-2-oxo-ethyl)amino]-1-oxopropan-2-yl}-1,4,7,10-tetraazacyclododecane-1,4,7-triyl)triacetate

To a degased solution of (3S)-3-(5-bromopyridin-3-yl)-3-[({(3R)-1-[3-(piperidin-4-yl)propanoyl]piperidin-3-yl}carbonyl)amino]propanoic acid (45 mg, 90 μmol), 1-aminobutane (135 μL, 1.36 mmol), copper(I)iodide (2.6 mg, 14 μmol) and tetrakis(triphenylphoshine)palladium(0) (10.5 mg, 9 μmol) in DMF (300 μL) was added gadolinium 2,2′,2″{10-[1-({2-[(but-3-yn-1-yl)oxy]-2-oxoethyl}amino)-1-oxopropan-2-yl]-1,4,7,10-tetraazacyclododecane-1,4,7-triyl}triacetate (115 mg, 120 μmol) in DMF (1 mL) at 100° C. over 1 hour. After 20 minutes the cooled reaction mixture was diluted with DMSO (1 ml) and purified by preparative HPLC (C18-YMC ODS AQ-10 μm, acetonitrile in water+0.1% formic acid 1% to 40%) to yield 7.9 mg of the title compound.

UPLC (ACN-HCOOH polar): Rt.=0.74 min.

MS (ES⁻): m/e=1095.8 (M−H⁺).

### Example 2

### Gadolinium 2,2′,2″-(10-{(2S)-1-[(2-{[4-(5-{(1S)-2-carboxy-1-[({(3R)-1-[3-(piperidin-4-yl)-propanoyl]piperidin-3-yl}carbonyl)amino]ethyl}pyridin-3-yl)but-3-yn-1-yl]amino}-2-oxo-ethyl)amino]-1-oxopropan-2-yl}-1,4,7,10-tetraazacyclododecane-1,4,7-triyl)triacetate

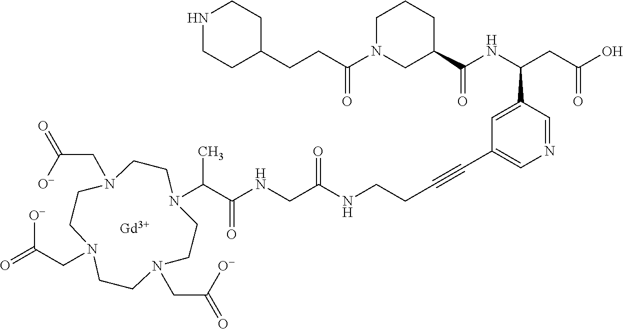

### Example 2a

### Gadolinium 2,2′,2″-{10-[1-({2-[(but-3-yn-1-yl)amino]-2-oxoethyl}amino)-1-oxopropan-2-yl]-1,4,7,10-tetraazacyclododecane-1,4,7-triyl}triacetate

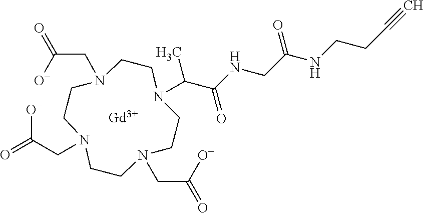

To gadolinium pyridinium 2,2′,2″-(10-{1-[(carboxylatomethyl)amino]-1-oxopropan-2-yl}-1,4,7,10-tetraazacyclododecane-1,4,7-triyl)triacetate (337 mg, 0.48 mmol) and N-ethyldiisopropylamine (500 μL, 2.6 mmol) in DMF (5 mL) and DMSO (5 mL) was added a solution of but-3-yn-1-yl amine hydrochloride (200 mg, 1.9 mmol) and N-ethyldiisopropyl amine (600 μL, 3.1 mmol) in DMF (2 mL) and DMSO (2 mL). HATU (253 mg, 0.67 mmol) was added as a solid and the mixture was stirred for 20 hours at room temperature. A mixture of water and ethyl acetate was added, the phases were separated and the organic phase was extracted with water. The aqueous phase was concentrated under reduced pressure and purified by preparative HPLC (C18-YMC ODS AQ-10 μm, acetonitrile in water+0.1% formic acid, 1% to 40%) to yield 126 mg of gadolinium 2,2′,2″-{1-[1-({2-[(but-3-yn-1-yl)amino]-2-oxoethyl}amino)-1-oxopropan-2-yl]-1,4,7,10-tetraazacyclododecane-1,4,7-triyl}triacetate.

UPLC (ACN-HCOOH polar): Rt.=0.48 min.

MS (ES): m/e=679.8 (M−H⁺).

### Example 2b

### Gadolinium 2,2′,2″-(10-{(2S)-1-[(2-{[4-(5-{(1S)-2-carboxy-1-[({(3R)-1-[3-(piperidin-4-yl)-propanoyl]piperidin-3-yl}carbonyl)amino]ethyl}pyridin-3-yl)but-3-yn-1-yl]amino}-2-oxoethyl)amino]-1-oxopropan-2-yl}-1,4,7,10-tetraazacyclododecane-1,4,7-triyl)tri-acetate

To a degased solution of (3S)-3-(5-bromopyridin-3-yl)-3-[({(3R)-1-[3-(piperidin-4-yl)propanoyl]piperidin-3-yl}carbonyl)amino]propanoic acid (60 mg, 120 μmol), piperidine (103 mg, 1.2 mmol), copper(I)iodide (3.4 mg, 18 μmol) and tetrakis(triphenylphoshine)palladium(0) (21 mg, 18 μmol) in DMF (2 mL) was added gadolinium 2,2′,2″-[10-(1-1-{[2-(but-3-yn-1-ylamino)-2-oxoethyl]amino}-1-oxopropan-2-yl)-1,4,7,10-tetraazacyclododecane-1,4,7-triyl]triacetate (206 mg, 680 μmol) in DMF (10 mL) at 100° C. over 2 hours. After 2 hours a mixture of water and ethyl acetate was added, the phases were separated and the organic phase was extracted with water. The aqueous phase was concentrated under reduced pressure and purified by preparative HPLC (C18-YMC ODS AQ-10 μm, acetonitrile in water+0.1% formic acid, 1% to 25%) to yield 7.0 mg of the title compound.

UPLC (ACN-HCOOH polar): Rt.=0.66 min.

MS (ES⁻): m/e=1094.5 (M−H⁺).

### Example 3

### Gadolinium 2,2′,2″-{10-[(2S)-1-({2-[(6-{4-[(5-{(1S)-2-carboxy-1-[({(3R)-1-[3-(piperidin-4- yl)propanoyl]piperidin-3-yl}carbonyl)amino]ethyl}pyridin-3-yl)ethynyl]phenyl}hexyl)-amino]-2-oxoethyl}amino)-1-oxopropan-2-yl]-1,4,7,10-tetraazacyclododecane-1,4,7-triyl}triacetate

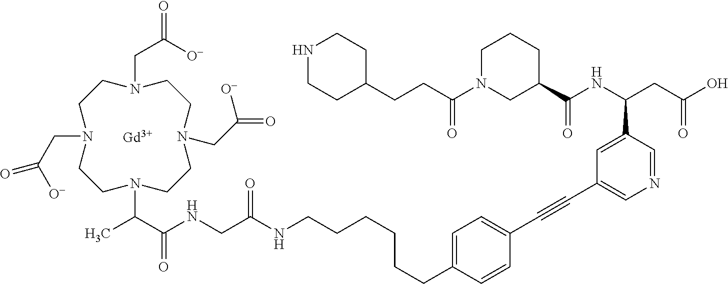

### Example 3a

### Methyl 6-(4-{[(trifluoromethyl)sulfonyl]oxy}phenyl)hexanoate

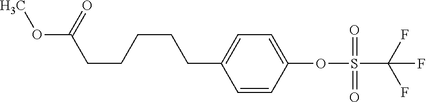 [SMILES: COC(=O)CCCCCc1ccc(OS(=O)(=O)C(F)(F)F)cc1]

To methyl 6-(4-hydroxyphenyl)hexanoate (Chu et al. Bioorg. Med. Chem. Lett. 2001, 11, 509-514, 1.95 g, 8.77 mmol) in pyridine (5 mL) was added trifluoromethane sulfonic anhydride (1.18 mL, 10.5 mmol) at 0° C. The mixture was stirred for 2 hours at 0° C. and for 17 hours at room temperature. A mixture of water and diethylether was added, the phases were separated and the aqueous phase was extracted with diethyl ether. Combined organic extracts were washed with 0.1 M hydrochloric acid, dried over sodium sulphate, concentrated under reduced pressure and the residue was purified by chromatography on silica gel (ethyl acetate in hexane, 0 to 30%) to yield 2.43 g of methyl 6-(4-{[(trifluoro-methyl)sulfonyl]oxy}phenyl)hexanoate.

¹H-NMR (400 MHz, CDCl₃): δ=1.32-1.43 (m, 2H), 1.59-1.73 (m, 4H), 2.32 (t, 2H), 2.64 (t, 2H), 3.67 (s, 3H), 7.18 (d, 2H), 7.24 (d, 2H) ppm.

### Example 3b

### Methyl 6-{4-[(trimethylsilyl)ethynyl]phenyl}hexanoate

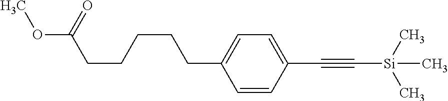

To methyl 6-(4-{[(trifluoromethyl)sulfonyl]oxy}phenyl)hexanoate 500 mg, 1.41 mmol), dichloropalladium(II)bis(triphenylphosphane) (92 mg, 0.13 mmol), copper iodide (25 mg, 0.13 mmol) and N,N-diisopropylethylamine (0.86 mL, 4.9 mmol) in DMF (3.6 mL) was added ethynyl(trimethyl)silane (0.59 mL, 4.2 mmol) in DMF (1.2 mL) over one hour at 45° C. The mixture was stirred for 4 days while the ethynyl(trimethyl)silane (0.59 mL, 4.2 mmol) additions were repeated after the second and third day. A mixture of water and hexane was added, the phases were separated and the aqueous phase was extracted with hexane. Combined organic extracts were washed with brine, dried over sodium sulphate, concentrated under reduced pressure and the residue was purified by chromatography on silica gel (ethyl acetate in hexane, 0 to 20%) to yield 153 mg of methyl 6-{4-[(trimethylsilyl)ethynyl]phenyl}hexanoate.

¹H-NMR (400 MHz, CDCl3): δ=0.25 (s, 9H), 1.22-1.40 (m, 2H), 1.57-1.71 (m, 4H), 2.30 (t, 2H), 2.60 (t, 2H), 3.67 (s, 3H), 7.10 (d, 2H), 7.38 (d, 2H) ppm.

### Example 3c

### 6-{4-[(Trimethylsilyl)ethynyl]phenyl}hexanal

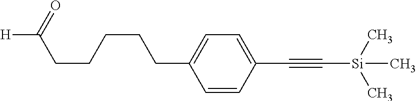 [SMILES: C[Si](C)(C)C#Cc1ccc(CCCCCC=O)cc1]

To methyl 6-{4-[(trimethylsilyl)ethynyl]phenyl}hexanoate (710 mg, 2.35 mmol) in diethyl ether (47 mL) was added a 1.2 M solution diisobutyl aluminium hydride in toluene (1.88 mL, 2.82 mmol) at −90° C. The solution was stirred for 30 minutes at −90° and 90 minutes at −70° C. The addition of a 1.2 M solution diisobutyl aluminium hydride in toluene (0.9 mL, 1.08 mmol) was repeated at −80° C. and saturated aqueous tartaric acid was added after 2.5 hours at −70° C. After vigorous stirring the phases were separated and the aqueous phase was extracted with ethyl acetate. Combined organic extracts were washed with brine, dried over sodium sulphate, concentrated under reduced pressure and the residue was purified by chromatography on silica gel (ethyl acetate in hexane, 0 to 30%) to yield 250 mg of 6-{4-[(trimethylsilyl)ethynyl]phenyl}hexanal.

¹H-NMR (400 MHz, CDCl3): δ=0.25 (s, 9H), 1.34-1.38 (m, 2H), 1.58-1.73 (m, 4H), 2.42 (t, 2H), 2.61 (t, 2H), 7.10 (d, 2H), 7.38 (d, 2H), 9.76 (s, 1H) ppm.

### Example 3d

### 6-{4-[(Trimethylsilyl)ethynyl]phenyl}hexan-1-amine

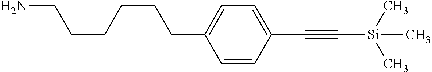 [SMILES: C[Si](C)(C)C#Cc1ccc(CCCCCCN)cc1]

To 6-{4-[(trimethylsilyl)ethynyl]phenyl}hexanal (240 mg, 0.88 mmol) in methanol (12 mL) was added ammonium acetate (340 mg, 4.4 mmol) and acetic acid (0.1 mL, 1.76 mmol). The solution was stirred for 10 minutes, 5-ethyl-2-methylpyridine borane complex (66 μL, 0.44 mmol) was added and stirring was continued for 16 hours. The solution was concentrated under reduced pressure and the residue was purified by chromatography on amino phase silica gel (ethyl acetate in hexane, 0 to 100% then methanol in ethyl acetate 0 to 20%) to yield 800 mg of 6-{4-[(trimethylsilyl)ethynyl]phenyl}hexan-1-amine.

¹H-NMR (400 MHz, CDCl3): δ=0.25 (s, 9H), 1.14-1.48 (m, 6H), 1.60 (quin, 2H), 2.59 (t, 2H), 2.67 (t, 2H), 6.96-7.16 (m, 2H), 7.31-7.51 (m, 2H) ppm.

### Example 3e

### Gadolinium 2,2′,2″-(10-{1-[(2-{[6-(4-ethynylphenyl)hexyl]amino}-2-oxoethyl)amino]-1-oxopropan-2-yl}-1,4,7,10-tetraazacyclododecane-1,4,7-triyl)triacetate

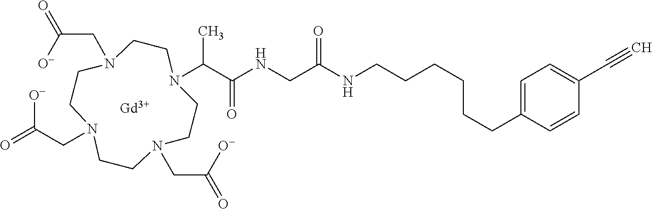

To gadolinium pyridinium 2,2′,2″-(10-{1-[(carboxylatomethyl)amino]-1-oxopropan-2-yl}-1,4,7,10-tetraazacyclododecane-1,4,7-triyl)triacetate (1.49 g, 2.11 mmol) and N-ethyldiisopropylamine (1.5 mL, 7.8 mmol) in DMF (23 mL) and DMSO (23 mL) was added a solution of 6-{4-[(trimethylsilyl)ethynyl]phenyl}hexan-1-amine (330 mg, 1.9 mmol) and N-ethyldiisopropylamine (1.0 mL, 5.2 mmol) in DMF (15 mL) and DMSO (15 mL) and the mixture was stirred for 5 minutes. HATU (688 mg, 1.81 mmol) was added as a solid and the mixture was stirred for 17 hours at room temperature. Water was added and the reaction mixture was washed with diethyl ether. The aqueous phase was concentrated under reduced pressure and the residue was solved in water (50 mL) and formic acid (46 μL). After 2 days a solid had precipitated, which was filtered off, the filtrate lyophilized and purified by preparative HPLC (C18-Chromatorex-10 μm, acetonitrile in water+0.1% formic acid, 15% to 55%) to yield 231 mg of gadolinium 2,2′,2″-(10-{1-[(2-{[6-(4-ethynylphenyl)hexyl]amino}-2-oxoethyl)amino]-1-oxopropan-2-yl}-1,4,7,10-tetraazacyclododecane-1,4,7-triyl)triacetate.

UPLC (ACN-HCOOH): Rt.=0.89 min.

MS (ES⁺): m/e=814.2 (M+H⁺).

MS (ES⁻): m/e=812.3 (M−H⁺).

### Example 3f

### Gadolinium 2,2′,2″-{10-[(2S)-1-({2-[(6-{4-[(5-{(1S)-2-carboxy-1-[({(3R)-1-[3-(piperidin-4- yl)propanoyl]piperidin-3-yl}carbonyl)amino]ethyl}pyridin-3-yl)ethynyl]phenyl}hexyl)-amino]-2-oxoethyl}amino)-1-oxopropan-2-yl]-1,4,7,10-tetraazacyclododecane-1,4,7-triyl}triacetate

To a degased solution of (3S)-3-(5-bromopyridin-3-yl)-3-[({(3R)-1-[3-(piperidin-4-yl)propanoyl]piperidin-3-yl}carbonyl)amino]propanoic acid (33 mg, 70 μmol), triethylamine (70 μL, 53 μmol), and {2-[(dimethylamino)methyl]phenyl}palladium(1)chloride-1,3,5-triaza-7-phosphatricyclo[3.3.1.1]decane (Organometallics 2006, 25, 5768-5773, 3.6 mg, 8 μmol) in water (0.9 mL) and acetonitrile (2.1 mL), which was stirred for 30 minutes at room temperature was added gadolinium 2,2′,2″-(10-{1-[(2-{[6-(4-ethynylphenyl)hexyl]amino}-2-oxoethyl)amino]-1-oxopropan-2-yl}-1,4,7,10-tetraazacyclododecane-1,4,7-triyl)triacetate (60 mg, 73 μmol) and the mixture was heated at 80° C. for 5.5 hours. The mixture was condensed after addition of water and the residue was purified by preparative HPLC (C18-Chromatorex-10 μm, acetonitrile in water+0.1% formic acid, 1% to 35%) to yield 4.4 mg of the title compound.

UPLC (ACN-HCOOH polar): Rt.=1.27 min.

MS (ES⁻): m/e=1226.7 (M−H)⁻.

### Example 4

### Gadolinium 2,2′,2″-{10-[(2S)-1-({2-[(6-{4-[(5-{(1S)-2-carboxy-1-[({(3R)-1-[3-(piperidin-4- yl)propanoyl]piperidin-3-yl}carbonyl)amino]ethyl}pyridin-3-yl)ethyl]phenyl}hexyl)-amino]-2-oxoethyl}amino)-1-oxopropan-2-yl]-1,4,7,10-tetraazacyclododecane-1,4,7-triyl}triacetate

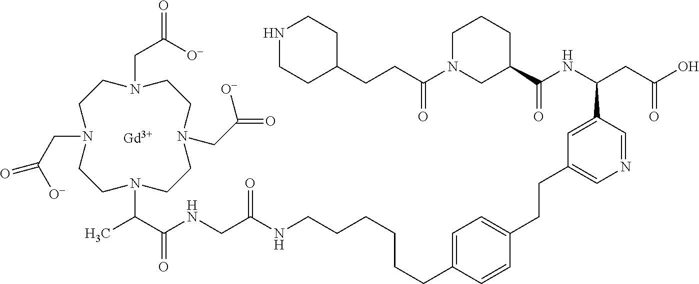

Gadolinium 2,2′,2″-{10-[(2S)-1-({2-[(6-{4-[(5-{(1S)-2-carboxy-1-[({(3R)-1-[3-(piperidin-4- yl)propanoyl]piperidin-3-yl}carbonyl)amino]ethyl}pyridin-3-yl)ethynyl]phenyl}hexyl)-amino]-2-oxo-ethyl}amino)-1-oxopropan-2-yl]-1,4,7,10-tetraazacyclododecane-1,4,7-triyl}triacetate (1.93 mg, 1.6 μmol) in ethanol (0.6 mL) and water (60 μL) was stirred under a hydrogen atmosphere for in the presence of palladium on charcoal (10%, 0.1 mg) for 20 hours. The mixture was diluted with ethanol and water and filtered. The filtrate was condensed under vacuum to yield 1.1 mg of the title compound.

UPLC (ACN-HCOOH polar): Rt.=1.10 min.

MS (ES⁻): m/e=1229.2 (M−H)⁻.

### Example 5

### Gadolinium 2,2′,2″-{10-[(2S)-1-({2-[(6-(3-[(5-{(1S)-2-carboxy-1-[({(3R)-1-[3-(piperidin-4- yl)propanoyl]piperidin-3-yl}carbonyl)amino]ethyl}pyridin-3-yl)ethynyl]phenyl}butyl)-amino]-2-oxoethyl)amino)-1-oxopropan-2-yl]-1,4,7,10-tetraazacyclododecane-1,4,7-triyl}triacetate

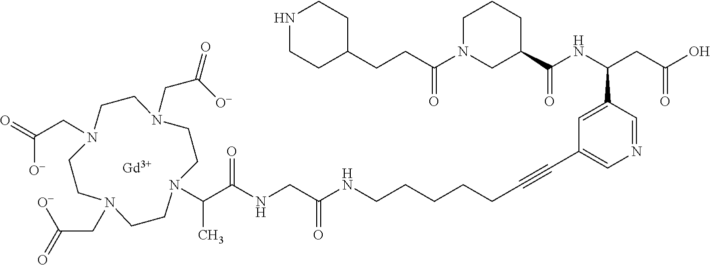

### Example 5a

### 2-[4-(3-Hydroxyphenyl)butyl]-1H-isoindole-1,3(2H)-dione

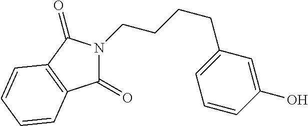 [SMILES: O=C1c2ccccc2C(=O)N1CCCCc1cccc(O)c1]

3,5-Dibromophenol (6.0 g, 23.8 mmol), 2-(but-3-en-1-yl)-1H-isoindole-1,3(2H)-dione (9.8 g, 49 mmol), palladium(II)acetate (53 mg, 0.24 mmol) and tris(2-methylphenyl)phosphane (145 mg, 0.48 mmol) were stirred in acetonitrile (125 mL) and triethylamine (6.6 mL) for 5 hours at 90° C. After stirring for 15 hours at room temperature and concentration a mixture of 2-[4-(3-bromo-5-hydroxyphenyl)but-3-en-1-yl]-1H-isoindole-1,3(2H)-dione and 2,2′-[(5-hydroxy-benzene-1,3-diyl)dibut-1-ene-1,4-diyl]bis(1H-isoindole-1,3(2H)-dione) was obtained, which could be separated by chromatography on silica gel (ethyl acetate in hexane, 0 to 30%) to yield 3.47 g of the bromo intermediate. The 2-[4-(3-bromo-5-hydroxyphenyl)but-3-en-1-yl]-1H-isoindole-1,3(2H)-dione was solved in methanol (230 mL), water (18 mL) and ethyl acetate (192 mL) and stirred under a hydrogen atmosphere for in the presence of palladium on charcoal (10%, 437 mg) at 40° C. for 2.5 hours. The reaction mixture was filtered through a path of celite, concentrated under reduced pressure and the residue was purified by chromatography on silica gel (ethyl acetate in hexane, 0 to 60%) to yield 2.41 g of 2-[4-(3-hydroxyphenyl)butyl]-1H-isoindole-1,3(2H)-dione.

¹H-NMR (300 MHz, DMSO-d₆): δ=1.41-1.67 (m, 4H), 2.47 (m, 2H), 3.58 (t, 2H), 6.47-6.65 (m, 3H), 6.96-7.10 (t, 1H), 7.76-7.92 (m, 4H), 9.21 (s, 1H) ppm.

### Example 5b

### 3-[4-(1,3-Dioxo-1,3-dihydro-2H-isoindol-2-yl)butyl]phenyl trifluoromethanesulfonate

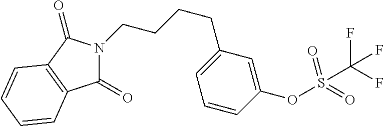 [SMILES: O=C1c2ccccc2C(=O)N1CCCCc1cccc(OS(=O)(=O)C(F)(F)F)c1]

To 2-[4-(3-hydroxyphenyl)butyl]-1H-isoindole-1,3(2H)-dione (4.24 g, 14.4 mmol) in pyridine (30 mL) was added trifluoromethane sulfonic anhydride (3.2 mL, 18.7 mmol) at 0° C. The mixture was stirred for one hour at 0° C., a mixture of water and diethyl ether was added, the phases were separated and the aqueous phase was extracted with diethyl ether. Combined organic extracts were washed with 0.5 M hydrochloric acid, dried over sodium sulphate. The solution was concentrated under reduced pressure while toluene was added two times before the end of the distillation and the residue was purified by chromatography on silica gel (ethyl acetate in hexane, 0 to 70%) to yield 5.34 g of 3-[4-(1,3-dioxo-1,3-dihydro-2H-isoindol-2-yl)butyl]phenyl trifluoromethanesulfonate.

¹H-NMR (300 MHz, DMSO-d₆): δ=1.50-1.69 (m, 4H), 2.68 (t, 2H), 3.55-3.67 (t, 2H), 7.22-7.38 (m, 3H), 7.46 (t, 1 H), 7.77-7.94 (m, 4H) ppm.

### Example 5c

### 2-(4-{3-[(Trimethylsilyl)ethynyl]phenyl}butyl)-1H-isoindole-1,3(2H)-dione

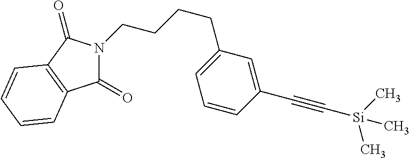 [SMILES: C[Si](C)(C)C#Cc1cccc(CCCCN2C(=O)c3ccccc3C2=O)c1]

To 3-[4-(1,3-dioxo-1,3-dihydro-2H-isoindol-2-yl)butyl]phenyl trifluoromethanesulfonate (5.3 g, 12.5 mmol), dichloropalladium(II)bis(triphenylphosphane) (440 mg, 0.63 mmol), copper iodide (120 mg, 0.63 mmol) and N,N-diisopropylethylamine (11 mL, 63 mmol) in DMF (20 mL) was added ethynyl(trimethyl)silane (8.7 mL, 63 mmol) in DMF (11 mL) over 11 hours at 50° C. The mixture was stirred for 25 hours at 50° C. while the ethynyl(trimethyl)silane (4.4 mL, 32 mmol) addition in DMF (5.6 mL) was repeated after 18 hours. A mixture of water and diethyl ether was added, the phases were separated and the aqueous phase was extracted with diethyl ether. Combined organic extracts were washed with brine, dried over sodium sulphate, concentrated under reduced pressure and the residue was purified by chromatography on silica gel (ethyl acetate in hexane, 0 to 25%) to yield 3.86 g of 2-(4-{3-[(trimethylsilyl)ethynyl]phenyl}butyl)-1H-isoindole-1,3(2H)-dione.

¹H-NMR (400 MHz, DMSO-d₆): δ=0.22 (s, 19H), 1.48-1.65 (m, 4H), 2.59 (t, 2H), 3.59 (t, 2H), 7.17-7.33 (m, 4H), 7.75-7.91 (m, 4H) ppm.

### Example 5d

### 4-{3-[(Trimethylsilyl)ethynyl]phenyl}butan-1-amine

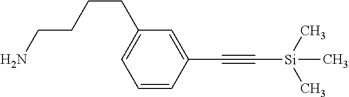 [SMILES: C[Si](C)(C)C#Cc1cccc(CCCCN)c1]

To 2-(4-{3-[(trimethylsilyl)ethynyl]phenyl}butyl)-1H-isoindole-1,3(2H)-dione (3.86 g, 10.3 mmol) in THF (83 mL) was added methyl hydrazine (8.1 mL, 15.4 mmol) and the solution was stirred for 41 hours at 40° C. while a precipitate formed. The reaction mixture was concentrated to a volume of 40 mL and filtered at 0° C. The solid was washed with a small amount of cold THF and the combined filtrates concentrated under reduced pressure while toluene was added two times before the end of the distillation to yield 2.63 g of 4-{3-[(trimethylsilyl)ethynyl]phenyl}butan-1-amine.

¹H-NMR (300 MHz, DMSO-d₆): δ=0.22 (s, 9H), 1.26-1.41 (m, 2H), 1.47-1.64 (m, 2H), 2.52-2.62 (m, 4H), 7.10-7.33 (m, 4H) ppm.

### Example 5f

### Gadolinium 2,2′,2″-(10-{1-[(2-{[4-(3-ethynylphenyl)butyl]amino}-2-oxoethyl)amino]-1-oxopropan-2-yl}-1,4,7,10-tetraazacyclododecane-1,4,7-triyl)triacetate

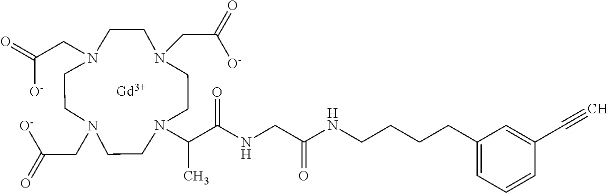

To gadolinium pyridinium 2,2′,2″-(10-{1-[(carboxylatomethyl)amino]-1-oxopropan-2-yl}-1,4,7,10-tetraazacyclododecane-1,4,7-triyl)triacetate (760 mg, 1.07 mmol), 4-{3-[(trimethylsilyl)ethynyl]phenyl}butan-1-amine (300 mg, 0.61 mmol) and N-ethyldiisopropyl amine (1.31 mL, 8.0 mmol) in DMF (19.5 mL) and DMSO (19.5 mL) was added HATU (348 mg, 0.92 mmol) as a solid and the mixture was stirred for 17 hours at room temperature. The mixture was condensed solved in DMF (5 mL), treated with TBAF (1 M, 0.37 mL) for 22 hours and purified by preparative HPLC (C18-YMC ODS AQ-10 μm, acetonitrile in water+0.1% formic acid, 1% to 55%) to yield 99 mg of gadolinium 2,2′,2″-(10-{1-[(2-{[4-(3-ethynylphenyl)butyl]amino}-2-oxoethyl)amino]-1-oxopropan-2-yl}-1,4,7,10-tetraazacyclododecane-1,4,7-triyl)triacetate.

UPLC (ACN-HCOOH): Rt.=0.77 min

MS (ES⁺): m/e=785.1 (M+H)⁺.

### Example 5g

### Gadolinium 2,2′,2″-{10-[(2S)-1-({2-[(6-{3-[(5-{(1S)-2-carboxy-1-[({(3R)-1-[3-(piperidin-4- yl)propanoyl]piperidin-3-yl}carbonyl)amino]ethyl}pyridin-3-yl)ethynyl]phenyl}butyl)-amino]-2-oxoethyl}amino)-1-oxopropan-2-yl]-1,4,7,10-tetraazacyclododecane-1,4,7-triyl}triacetate

To a degased solution of (3S)-3-(5-bromopyridin-3-yl)-3-[({(3R)-1-[3-(piperidin-4-yl)propanoyl]piperidin-3-yl}carbonyl)amino]propanoic acid (31 mg, 62 μmol), triethylamine (70 μL, 0.5 mmol), and {2-[(dimethylamino)methyl]phenyl}palladium(I)chloride-1,3,5-triaza-7-phosphatricyclo[3.3.1.1]decane (2.7 mg, 6.2 μmol) in water (1.0 mL) and acetonitrile (2.5 mL), which was stirred for 30 minutes at room temperature was added gadolinium 2,2′,2″-(10-{1-[(2-{[4-(3-ethynylphenyl)butyl]amino}-2-oxoethyl)amino]-1-oxopropan-2-yl}-1,4,7,10-tetraazacyclododecane-1,4,7-triyl)triacetate (55 mg, 70 μmol) and the mixture was heated at 80° C. for 6.5 hours. The mixture was condensed after addition of water and the residue was purified by preparative HPLC (C18-YMC ODS AQ-10 μm, acetonitrile in water+0.1% formic acid, 1% to 40%) to yield 2.8 mg of the title compound.

UPLC (ACN-HCOOH polar): Rt.=1.14 min.

MS (ES⁻): m/e=1198.2 (M−H)⁻.

### Example 6

### Digadolinium 2,2′,2″,2′″,2″″,2′″″-({5-[(5-{(1S)-2-carboxy-1-[({(3R)-1-[3-(piperidin-4-yl)-propanoyl]piperidin-3-yl}carbonyl)amino]ethyl}pyridin-3-yl)ethynyl]-1,3-phenylene}-bis[butane-4,1-diylimino(2-oxoethane-2,1-diyl)imino(1-oxopropane-1,2-diyl)-1,4,7,10-tetraazacyclododecane-10,1,4,7-tetrayl])hexaacetate

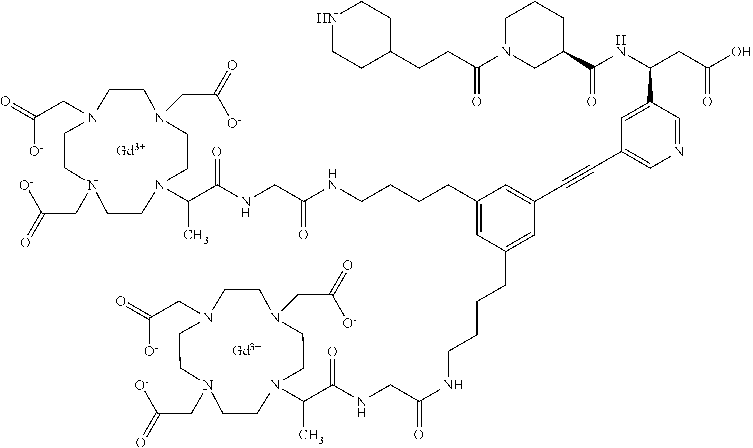

### Example 6a

### 2,2′-[(5-Hydroxybenzene-1,3-diyl)dibutane-4,1-diyl]bis(1H-isoindole-1,3(2H)-dione)

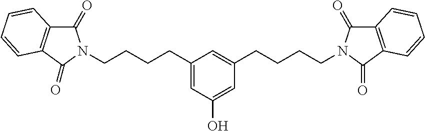 [SMILES: O=C1c2ccccc2C(=O)N1CCCCc1cc(O)cc(CCCCN2C(=O)c3ccccc3C2=O)c1]

2,2′-[(5-hydroxybenzene-1,3-diyl)dibut-1-ene-1,4-diyl]bis(1H-isoindole-1,3(2H)-dione (example 5a, 1.75 g, 3.55 mmol) was solved in methanol (88 mL), water (19 mL) and ethyl acetate (71 mL) and stirred under a hydrogen atmosphere for in the presence of palladium on charcoal (10%, 166 mg) at 40° C. for 3.5 hours. The reaction mixture was filtered through celite, concentrated under reduced pressure and the residue was purified by chromatography on silica gel (ethyl acetate in hexane, 0 to 60%) to yield 1.0 g of 2,2′-[(5-hydroxybenzene-1,3-diyl)dibutane-4,1-diyl]bis(1H-isoindole-1,3(2H)-dione).

¹H-NMR (300 MHz, DMSO-d₆): δ=1.39-1.66 (m, 8H), 2.44 (t, 4H), 3.57 (t, 4H), 6.29-6.47 (m, 3H), 7.75-7.89 (m, 8H), 9.05 (s, 1H) ppm.

### Example 6b

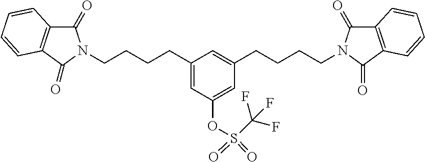 [SMILES: O=C1c2ccccc2C(=O)N1CCCCc1cc(CCCCN2C(=O)c3ccccc3C2=O)cc(OS(=O)(=O)C(F)(F)F)c1]

To 2,2′-[(5-hydroxybenzene-1,3-diyl)dibutane-4,1-diyl]bis(1H-isoindole-1,3(2H)-dione) (6.46 g, 12.9 mmol) in pyridine (14.6 mL) was added trifluoromethane sulfonic anhydride (2.6 mL, 15.6 mmol) at 0° C. The mixture was stirred for 30 minutes at 0° C. and 3 hours at room temperature. After additional addition of trifluoromethane sulfonic anhydride (0.52 mL) at 0° C. a mixture of water and diethyl ether was added after 1.5 hours, the phases were separated and the aqueous phase was extracted with diethyl ether. Combined organic extracts were washed with 0.5 M hydrochloric acid, dried over sodium sulphate. The solution was concentrated under reduced pressure while toluene was added two times before the end of the distillation and the residue was purified by chromatography on silica gel (ethyl acetate in hexane, 0 to 50%) to yield 7.4 g of 3,5-bis[4-(1,3-dioxo-1,3-dihydro-2H-isoindol-2-yl)butyl]phenyl trifluoromethanesulfonate.

¹H-NMR (300 MHz, DMSO-d₆): δ=1.43-1.68 (m, 8H), 2.54-2.68 (m, 4H), 3.52-3.66 (m, 4H), 6.99-7.24 (m, 3H), 7.73-7.91 (m, 8H) ppm.

### Example 6c

### 2,2′-({5-[(Trimethylsilyl)ethynyl]benzene-1,3-diyl}dibutane-4,1-diyl)bis(1H-isoindole-1,3(2H)-dione)

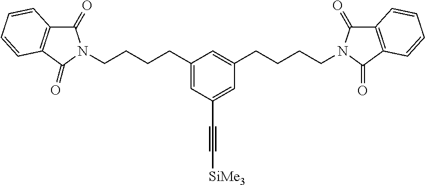 [SMILES: C[Si](C)(C)C#Cc1cc(CCCCN2C(=O)c3ccccc3C2=O)cc(CCCCN2C(=O)c3ccccc3C2=O)c1]

To 3,5-bis[4-(1,3-dioxo-1,3-dihydro-2H-isoindol-2-yl)butyl]phenyl trifluoromethanesulfonate 7.4 g, 11.8 mmol), dichloropalladium triphenylphosphane (413 mg, 0.59 mmol), copper iodide (112 mg, 0.59 mmol) and N,N-diisopropylethylamine (10.3 mL, 59 mmol) in DMF (30 mL) was added ethynyl(trimethyl)silane (13.3 mL, 118 mmol) in DMF (11 mL) over 15 hours at 50° C. The mixture was stirred for 4 hours at 50° C. Dichloropalladium triphenylphosphane (413 mg, 0.59 mmol) and copper iodide (112 mg, 0.59 mmol) were added as a solid and the ethynyl(trimethyl)silane (16.3 mL, 118 mmol) addition in DMF (5.6 mL) was repeated analogously. A mixture of water and diethyl ether was added, the phases were separated and the aqueous phase was extracted with diethyl ether. Combined organic extracts were washed with brine, dried over sodium sulphate, concentrated under reduced pressure and the residue was purified by chromatography on silica gel (ethyl acetate in hexane, 0 to 50%) to yield 3.18 g of 2,2′-({5-[(trimethylsilyl)ethynyl]benzene-1,3-diyl}dibutane-4,1-diyl)bis(1H-isoindole-1,3(2H)-dione).

¹H-NMR (300 MHz, DMSO-d₆): δ=0.14-0.31 (m, 9H), 1.56 (d, 8H), 2.55 (br. s., 3H), 3.57 (t, 4H), 6.94-7.13 (m, 3H), 7.70-7.93 (m, 9H) ppm.

### Example 6d

### 4,4′-(5-Ethynylbenzene-1,3-diyl)dibutan-1-amine

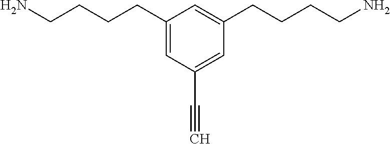 [SMILES: C#Cc1cc(CCCCN)cc(CCCCN)c1]

To 2,2′-({5-[(trimethylsilyl)ethynyl]benzene-1,3-diyl}dibutane-4,1-diyl)bis(1H-isoindole-1,3-(2H)-dione) (2.0 g, 3.47 mmol) in THF (42 mL) was added methyl hydrazine (3.65 mL, 69 mmol, 0.9 mL after 3 hours) and the solution was stirred for 17 hours at 40° C. while a precipitate formed. The reaction mixture was filtered at 0° C. and the solid washed with a small amount of cold THF. The combined filtrates were concentrated under reduced pressure while toluene was added two times before the end of the distillation to yield 1.08 g 4,4′-(5-ethynylbenzene-1,3-diyl)dibutan-1-amine.

¹H-NMR (300 MHz, DMSO-d₆): δ=1.35 (br., 4H), 1.56 (br., 4H), 2.55 (br., 4H), 3.11-3.54 (br, 4H), 7.01-7.14 (m, 3H) ppm.

### Example 6e Digadolinium 2,2′,2″,2′″,2″″,2′″″-{(5-ethynyl-1,3-phenylene)bis[butane-4,1-diylimino (2-oxoethane-2,1-diyl)imino(1-oxopropane-1,2-diyl)-1,4,7,10-tetraazacyclododecane-10,1,4,7-tetrayl]}hexaacetate

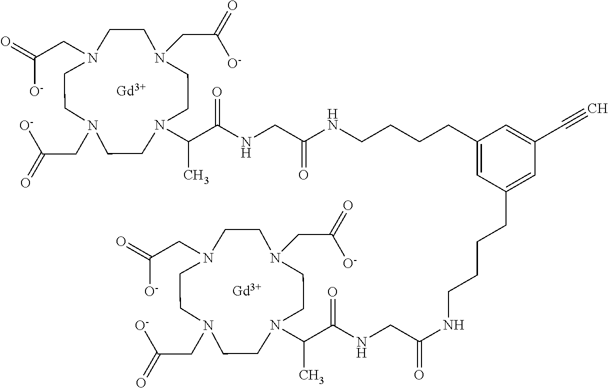

To gadolinium pyridinium 2,2′,2″-(10-{1-[(carboxylatomethyl)amino]-1-oxopropan-2-yl}-1,4,7,10-tetraazacyclododecane-1,4,7-triyl)triacetate (1015 mg, 1.43 mmol) and N-ethyldiisopropyl amine (1.0 mL, 6.0 mmol) in DMF (12 mL) and DMSO (12 mL) was added HATU (348 mg, 0.92 mmol) as a solid and the mixture was stirred for 4 minutes at room temperature. Then 4,4′-(5-ethynylbenzene-1,3-diyl)dibutan-1-amine (200 mg, 0.82 mmol) and N-ethyldiisopropyl amine (0.4 mL, 2.5 mmol) in DMF (8 mL) and DMSO (8 mL) were added and the mixture was stirred for 6 hours. The mixture was condensed and the residue was solved in water, which was washed with diethyl ether, and the condensed aqueous phase purified by preparative HPLC (C18-YMC ODS AQ-10 μm, acetonitrile in water+0.1% formic acid, 1% to 40%) to yield 111 mg of digadolinium 2,2′,2″,2′″,2″″,2′″″-{(5-ethynyl-1,3-phenylene)bis[butane-4,1-diylimino(2-oxoethane-2,1-diyl)imino(1-oxo propane-1,2-diyl)-1,4,7,10-tetraazacyclododecane-10,1,4,7-tetrayl]}hexaacetate.

UPLC (ACN-HCOOH polar): Rt.=1.03 min.

MS (ES⁻): m/e=1466.4 (M−H)⁻.

### Example 6f

### Digadolinium 2,2′,2″,2′″,2″″,2′″″-({5-[(5-{(1S)-2-carboxy-1-[({(3R)-1-[3-(piperidin-4-yl)-propanoyl]piperidin-3-yl}carbonyl)amino]ethyl}pyridin-3-yl)ethynyl]-1,3-phenylene}bis-[butane-4,1-diylimino(2-oxoethane-2,1-diyl)imino(1-oxopropane-1,2-diyl)-1,4,7,10-tetra-azacyclododecane-10,1,4,7-tetrayl])hexaacetate

To a degased solution of (3S)-3-(5-bromopyridin-3-yl)-3-[({(3R)-1-[3-(piperidin-4-yl)propanoyl]piperidin-3-yl}carbonyl)amino]propanoic acid (13.9 mg, 28 μmol), triethylamine (30 μL, 23 μmol), and {2-[(dimethylamino)methyl]phenyl}palladium(I)chloride-1,3,5-triaza-7-phosphatricyclo[3.3.1.1]decane (1.2 mg, 2.7 μmol) in water (0.3 mL) and acetonitrile (0.7 mL), which was stirred for 30 minutes at room temperature, was added digadolinium 2,2′,2″,2′″,2″″,2′″″-{(5-ethynyl-1,3-phenylene)bis[butane-4,1-diylimino(2-oxoethane-2,1-diyl)imino(1-oxo propane-1,2-diyl)-1,4,7,10-tetraazacyclododecane-10,1,4,7-tetrayl]} hexa acetate (59 mg, 40 μmol) in water (0.3 mL) and acetonitrile (0.7 mL) at 80° C. over two hours. The mixture was heated at 80° C. for additional 3 hours, after cooling to room temperature additional {2-[(dimethylamino)methyl]phenyl}palladium(I)chloride-1,3,5-triaza-7-phosphatricyclo[3.3.1.1]decane (2.4 mg, 5.4 μmol) and N-diisopropylethyl amine (30 μL) were added and heating at 80° C. was continued for 3 hours. The mixture was condensed and the residue purified by preparative HPLC (C18-Chromatorex-10 μm, acetonitrile in water+0.1% formic acid, 20% to 40%) to yield 3.4 mg of the title compound.

UPLC (ACN-HCOOH polar): Rt.=0.97-1.00 min.

MS (ES⁻): m/e=1881.2 (M−H)⁻.

### Example 7

### Tetragadolinium 2,2′,2″,2′″,2″″,2′″″,2″″″,2′″″″,2″″″″,2′″″″″,2″″″″″,2″″″″″″-({5-[(5-{(1S)-2-carboxy-1[({(3R)-1-[3-(piperidin-4-yl)propanoyl]piperidin-3-yl}carbonyl)amino]ethyl}-pyridin-3-yl)ethynyl]-1,3-phenylene}bis[butane-4,1-diylcarbamoyl(3,6,11,14-tetraoxo-4,7,10,13-tetraazahexadecane-8,2,15-triyl)di-1,4,7,10-tetraazacyclododecane-10,1,4,7-tetrayl])dodecaacetate

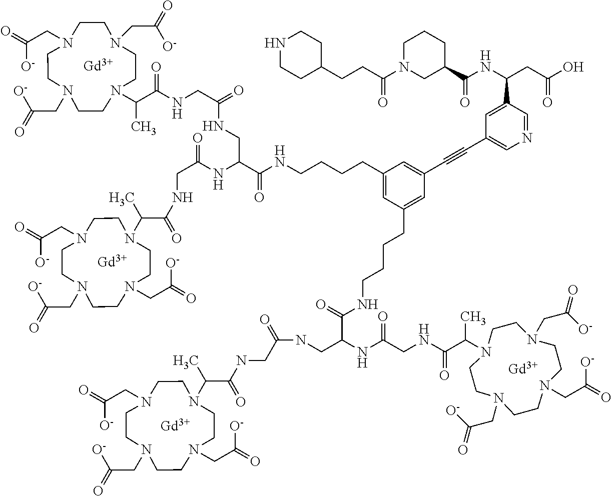

### Example 7a

### Tetra-tert-butyl {(5-ethynyl-1,3-phenylene)bis[butane-4,1-diylimino(3-oxopropane-3,1,2-triyl)]}tetrakiscarbamate

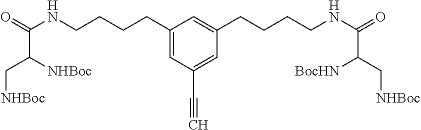 [SMILES: C#Cc1cc(CCCCNC(=O)C(CNC(=O)OC(C)(C)C)NC(=O)OC(C)(C)C)cc(CCCCNC(=O)C(CNC(=O)OC(C)(C)C)NC(=O)OC(C)(C)C)c1]

4,4′-(5-Ethynylbenzene-1,3-diyl)dibutan-1-amine (340 mg, 0.97 mmol) in DMF (3 mL) was added to a freshly prepared solution of N-(tert-butoxycarbonyl)-3-[(tert-butoxy carbonyl)-amino]alanine N-cyclohexylcyclohexanamine (1.04 g, 2.14 mmol), N,N-diisopropylethyl amine (1.0 mL, 5.8 mmol) and HATU (889 mg, 2.34 mmol) in DMF (9 mL) at 0° C. After stirring for 30 minutes the mixture was condensed and purified by chromatography on amino phase silica gel (ethyl acetate in hexane, 0 to 100%) to yield 340 mg of tetra-tert-butyl {(5-ethynyl-1,3-phenylene)bis[butane-4,1-diylimino(3-oxo propane-3,1,2-triyl)]}tetrakis carbamate.

¹H-NMR (400 MHz, CDCl₃): δ=1.35-1.53 (m, 36H), 1.49 (quin, 4H) 1.64 (quin, 4H), 1.72 (br, 4H), 2.59 (t, 4H), 3.02 (s, 1H), 3.13-3.33 (m, 4H), 3.38-3.56 (m, 4H), 4.11-4.22 (m, 2H), 5.29 (br., 2H), 5.87 (br., 2H), 6.99 (d, 1H), 7.13 (s, 2H) ppm.

### Example 7b

### 3,3′-[(5-Ethynyl-1,3-phenylene)bis(butane-4,1-diylimino)]bis(3-oxopropane-1,2-diaminium)tetrachloride

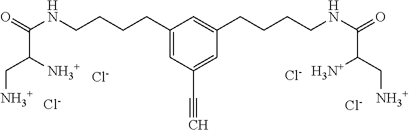 [SMILES: C#Cc1cc(CCCCNC(=O)C([NH3+])C[NH3+])cc(CCCCNC(=O)C([NH3+])C[NH3+])c1 ; [Cl-] ; [Cl-] ; [Cl-] ; [Cl-]]

To tetra-tert-butyl {(5-ethynyl-1,3-phenylene)bis[butane-4,1-diylimino(3-oxopropane-3,1,2-triyl)]}tetrakis carbamate (340 mg, 0.42 mmol) in DMF was added hydrochloric acid in dioxane (4M, 1.3 mL) The reaction vessel was sealed and irradiated in a microwave reactor for 18 minutes at 80° C. The addition of hydrochloric acid in dioxane (4M, 1.3 mL) and the microwave procedure were repeated once and the reaction mixture was diluted with 1,4-dioxane. The mixture was stirred while a precipitate formed which was collected by filtration to yield 194 mg of 3,3′-[(5-ethynyl-1,3-phenylene)bis(butane-4,1-diylimino)]bis(3-oxopropane-1,2-diaminium)tetrachloride.

¹H-NMR (300 MHz, DMSO-d₆): δ=1.36-1.53 (m, 4H), 1.55-1.70 (m, 4H), 2.52-2.61 (m, 4H), 3.01-3.27 (m, 8H), 4.10 (s, 1H), 4.23 (t, 2H), 7.13 (s, 3H), 8.64 (br., 12H), 8.88 (t, 2H) ppm.

### Example 7c

### Tetragadolinium 2,2′,2″,2′″,2″″,2′″″,2″″″,2″″″″,2′″″″″,2″″″″″,2′″″″″″-{(5-ethynyl-1,3-phenylene)bis[butane-4,1-diylcarbamoyl(3,6,11,14-tetraoxo-4,7,10,13-tetraaza hexa-decane-8,2,15-triyl)di-1,4,7,10-tetraazacyclododecane-10,1,4,7-tetrayl]}dodeca acetate

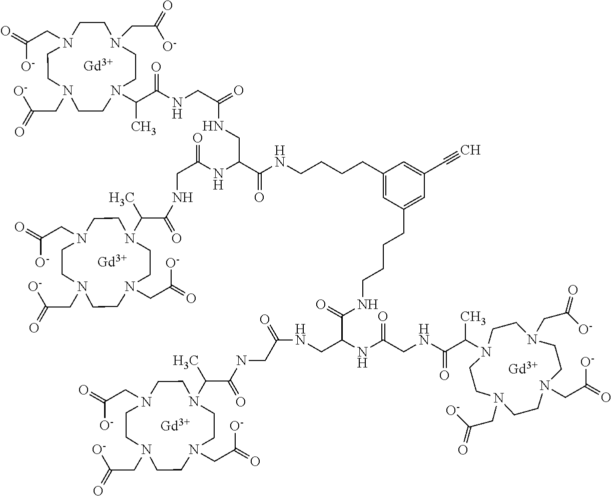

To gadolinium 2,2′,2″-[10-(1-{[2-(4-nitrophenoxy)-2-oxoethyl]amino}-1-oxopropan-2-yl)-1,4,7,10-tetraazacyclododecane-1,4,7-triyl]triacetate (WO 2001051095 A2, 66.8 mg, 90 μmol) and 3,3′-[(5-ethynyl-1,3-phenylene)bis(butane-4,1-diylimino)]bis(3-oxopropane-1,2-diaminium)tetrachloride (20 mg, 0.02 μmol) in DMSO (0.25 mL) was added triethylamine (74 μL, 0.53 mmol) and the mixture was stirred for 20 hours. Additional gadolinium 2,2′,2″-[10-(1-{[2-(4-nitrophenoxy)-2-oxoethyl]amino}-1-oxopropan-2-yl)-1,4,7,10-tetraazacyclo dodecane-1,4,7-triyl]triacetate (66 mg, 90 μmol) in DMSO (0.2 mL)was added and stirring was continued at 50° C. for 20 hours. The mixture was diluted with water and low molecular weight components were separated via ultrafiltration (cellulose acetate membrane, lowest NMWL 1000 g/mol, Millipore). The retentate was collected and purified by preparative HPLC (C18-YMC ODS AQ-10 μm, acetonitrile in water+0.1% formic acid, 1% to 40%) to yield 18.5 mg of Tetragadolinium 2,2′,2″,2′″,2″″,2′″″,2″″″,2′″″″,2″″″″,2′″″″″,2″″″″″,2′″″″″″-{(5-ethynyl-1,3-phenylene)bis[butane-4,1-diylcarbamoyl(3,6,11,14-tetraoxo-4,7,10,13-tetraaza hexadecane-8,2,15-triyl)di-1,4,7,10-tetraazacyclododecane-10,1,4,7-tetrayl]}dodeca acetate.

UPLC (ACN-HCOOH polar): Rt.=0.89-0.91 min.

MS (ES⁻): m/e=1430.6 (M−2H)²⁻.

### Example 7d

### Tetragadolinium 2,2′,2″,2′″,2″″,2′″″,2″″″,2′″″″,2″″″″,2′″″″″,2″″″″″,2′″″″″″-({5-[(5-{(1S)-2-carboxy-1-[({(3R)-1-[3-(piperidin-4-yl)propanoyl]piperidin-3-yl}carbonyl)amino]ethyl}-pyridin-3-yl)ethynyl]-1,3-phenylene}bis[butane-4,1-diylcarbamoyl(3,6,11,14-tetraoxo-4,7,10,13-tetraazahexadecane-8,2,15-triyl)di-1,4,7,10-tetraazacyclododecane-10,1,4,7-tetrayl])dodecaacetate

To a degased solution of (3S)-3-(5-bromopyridin-3-yl)-3-[({(3R)-1-[3-(piperidin-4-yl)propanoyl]piperidin-3-yl}carbonyl)amino]propanoic acid (9.3 mg, 19 μmol), N,N-diisopropyl ethyl amine (30 μL, 150 μmol), and {2-[(dimethylamino)methyl]phenyl}palladium(1)chloride-1,3,5-triaza-7-phosphatricyclo[3.3.1.1]decane (1.6 mg, 3.7 μmol) in water (0.3 mL) and acetonitrile (0.7 mL), which was stirred for 30 minutes at room temperature was added tetragadolinium 2,2′,2″,2′″,2″″,2′″″,2″″″,2′″″″,2″″″″,2′″″″″,2″″″″″,2″″″″″-{(5-ethynyl-1,3-phenylene)bis[butane-4,1-diylcarbamoyl(3,6,11,14-tetraoxo-4,7,10,13-tetraazahexa decane-8,2,15-triyl)di-1,4,7,10-tetraazacyclododecane-10,1,4,7-tetrayl]}dodeca acetate (63 mg, 22 μmol) in water (0.5 mL) and acetonitrile (1 mL) at 80° C. over two hours. The mixture was heated at 80° C. for additional 3 hours. After cooling to room temperature additional {2-[(dimethylamino)methyl]phenyl}palladium(I)chloride-1,3,5-triaza-7-phosphatricyclo[3.3.1.1]-decane (2.4 mg, 5.4 μmol) and N-diisopropylethyl amine (30 μL) were added and heating at 80° C. was continued for 3 hours. The mixture was condensed and the residue purified by preparative HPLC (C18-YMC ODS AQ-10 μm, acetonitrile in water+0.1% formic acid, 1% to 25%) to yield 3.2 mg of the title compound.

UPLC (ACN-HCOOH polar): Rt.=0.89-0.90 min.

MS (ES⁻): m/e=1637.1 (M−2H)²⁻.

### Example 8

### Tetragadolinium N-{2-[4,7,10-tris(carboxylatomethyl)-1,4,7,10-tetraazacyclo dodecan-1-yl]propanoyl}glycyl-3-[(N-{2-[4,7,10-tris(carboxylatomethyl)-1,4,7,10-tetraazacyclo-dodecan-1-yl]propanoyl}glycyl)amino]alanyl-N-(4-{3-[(5-{(1S)-2-carboxy-1-[({(3R)-1-[3-(piperidin-4-yl)propanoyl]piperidin-3-yl}carbonyl)amino]ethyl}pyridin-3-yl)ethynyl]-phenyl}butyl)-3-[(N-{2-[4,7,10-tris(carboxylatomethyl)-1,4,7,1 0-tetraazacyclododecan-1-yl]propanoyl}glycyl-3-[(N-{2-[4,7,10-tris(carboxylatomethyl)-1,4,7,10-tetraazacyclo-dodecan-1-yl]propanoyl}glycyl)amino]alanyl)amino]alaninamide

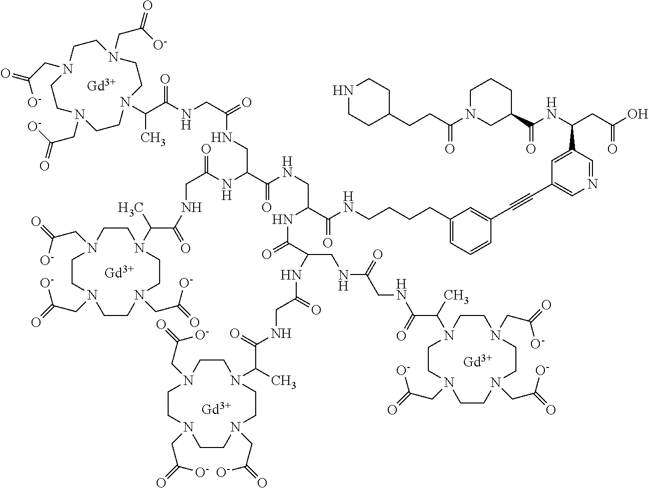

### Example 8a

### N-(tert-Butoxycarbonyl)-3-[(tert-butoxycarbonyl)amino]-N-(4-{3-[(trimethylsilyl)-ethynyl]phenyl}butyl)alaninamide

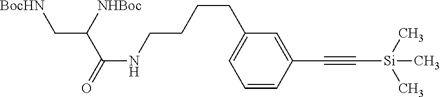 [SMILES: CC(C)(C)OC(=O)NCC(NC(=O)OC(C)(C)C)C(=O)NCCCCc1cccc(C#C[Si](C)(C)C)c1]

4-{3-[(Trimethylsilyl)ethynyl]phenyl}butan-1-amine (2.6 g, 9.7 mmol) in DMF (40 mL) was added to a freshly prepared solution of N-(tert-butoxycarbonyl)-3-[(tert-butoxy-carbonyl)amino]alanine N-cyclohexylcyclohexanamine (5.0 g, 10.2 mmol), N,N-diisopropylethylamine (8.2 mL, 48.7 mmol) and HATU (5.2 g, 13.6 mmol) in DMF (50 mL) at 0° C. After stirring for one hour the mixture was filtered cold, the filtrate condensed, while remaining traces of DMF were distilled in the presence of toluene, and purified by chromatography on amino phase silica gel (ethyl acetate in hexane, 0 to 40%) to yield 4.25 g of N-(tert-butoxycarbonyl)-3-[(tert-butoxycarbonyl)amino]-N-(4-{3-[(trimethylsilyl)ethynyl]phenyl}butyl)alaninamide.

¹H-NMR (300 MHz, DMSO-d₆): δ=0.22 (s, 9H), 1.35-1.43 (m, 2H), 1.36 (s, 18H), 1.44-1.60 (m, 2H), 2.92-3.21 (m, 4H), 3.93 (dd, 1H), 6.62 (d, 1H), 6.71 (t, 1H), 7.15-7.34 (m, 4H), 7.80 (t, 1H) ppm.

### Example 8b

### 3-Oxo-{3-[(4-O-[(trimethylsilyl)ethynyl]phenyl}butyl)amino]propane-1,2-diaminium dichloride

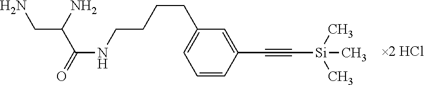 [SMILES: C[Si](C)(C)C#Cc1cccc(CCCCNC(=O)C(N)CN)c1]

N-(tert-butoxycarbonyl)-3-[(tert-butoxycarbonyl)amino]-N-(4-{3-[(trimethylsilyl)ethynyl]phenyl}butyl)alaninamide (4.28 g, 8.0 mmol) in DMF (18.5 mL) was added hydrochloric acid in dioxane (4M, 18 mL). The solution was divided into two pressure vessels, which were sealed and irradiated in a microwave reactor for 16 minutes at 80° C. The combined reaction solution was diluted with 1,4-dioxane (300 mL), condensed to a volume of 50 mL and again diluted with 1,4-dioxane (200 mL). The mixture was stirred while a precipitate formed which was collected by filtration to yield 1.77 g of 3-oxo-3-[(4-{3-[(trimethylsilyl)ethynyl]phenyl}butyl)amino]propane-1,2-diaminium dichloride.

¹H-NMR (300 MHz, DMSO-d₆): δ=0.22 (s, 9H), 1.46 (quin, 2H), 1.61 (m, 2H), 2.58 (t, 2H), 3.02-3.14 (m, 1H), 3.17-3.27 (m, 3H), 4.19 (t, 1H), 7.15-7.38 (m, 4H), 8.58 (br., 6H), 8.82 (t, 1H) ppm.

### Example 8c

### N-(tert-butoxycarbonyl)-3-[(tert-butoxycarbonyl)amino]alanyl-3-({N-(tert-butoxy-carbonyl)-3-[(tert-butoxycarbonyl)amino]alanyl}amino)-N-(4-{3-[(trimethylsilyl)ethynyl]-phenyl}butyl)alaninamide

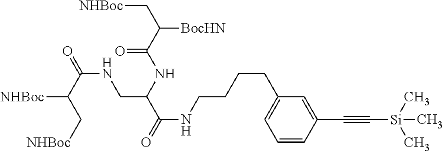 [SMILES: CC(C)(C)OC(=O)NCC(NC(=O)OC(C)(C)C)C(=O)NCC(NC(=O)C(CNC(=O)OC(C)(C)C)NC(=O)OC(C)(C)C)C(=O)NCCCCc1cccc(C#C[Si](C)(C)C)c1]

3-Oxo-3-[(4-{3-[(trimethylsilyl)ethynyl]phenyl}butyl)amino]propane-1,2-diaminium dichloride (1.77 g, 4.38 mmol) in DMF (40 mL) and N,N-diisopropylethylamine (4.4 mL) was added to a freshly prepared solution of N-(tert-butoxycarbonyl)-3-[(tert-butoxycarbonyl)amino]-alanine N-cyclohexyl cyclohexanamine (4.4 g, 9.19 mmol), N,N-diisopropylethylamine (14 mL) and HATU (4.66 g, 12.3 mmol) in DMF (50 mL) at 0° C. After stirring for 60 minutes the mixture was condensed and purified by chromatography on amino phase silica gel (ethyl acetate in hexane, 0 to 100%) to yield 3.37 g of N-(tert-butoxycarbonyl)-3-[(tert-butoxy-carbonyl)amino]alanyl-3-({N-(tert-butoxycarbonyl)-3-[(tert-butoxycarbonyl)amino]alanyl}-amino)-N-(4-{3-[(trimethylsilyl)ethynyl]phenyl}butyl)alanine amide.

¹H-NMR (400 MHz, DMSO-d₆): δ=0.23 (s, 9H), 1.39 (s, 36H), 1.46 (quin, 2H), 1.59 (quin, 2H), 2.58 (t, 2H), 3.08-3.38 (m, 6H), 3.91-4.09 (m, 2H), 4.19-4.36 (m, 1H), 6.19 (br, 1H), 6.31 (br, 1H), 6.45 (br, 1H), 7.14-7.32 (m, 4H), 7.45-7.69 (br, 2H) ppm.

### Example 8d

### 3-({(3-{[2,3-Diammoniopropanoyl]amino}-1-oxo-1-[(4-{3-[(trimethylsilyl)ethynyl]phenyl}butyl)amino]propan-2-yl}amino)-3-oxopropane-1,2-diaminium tetrachloride

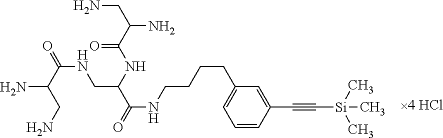 [SMILES: C[Si](C)(C)C#Cc1cccc(CCCCNC(=O)C(CNC(=O)C(N)CN)NC(=O)C(N)CN)c1]

To N-(tert-butoxycarbonyl)-3-[(tert-butoxycarbonyl)amino]alanyl-3-({N-(tert-butoxy carbonyl)-3-[(tert-butoxycarbonyl)amino]alanyl}amino)-N-(4-{3-[(trimethylsilyl)ethynyl]phenyl}butyl)alaninamide (4.48 g, 4.46 mmol) in DMF (21 mL) was added hydrochloric acid in dioxane (4M, 33 mL) The reaction vessel was sealed and irradiated in a microwave reactor for 10 minutes at 80° C. After cooling to room temperature the reaction mixture was slowly added to 1,4-dioxane (360 mL) while stirring. The formed precipitate was collected by filtration to yield 2.78 g of 3-({(3-{[2,3-diammoniopropanoyl]amino}-1-oxo-1-[(4-{3-[(trimethylsilyl)ethynyl]phenyl}butyl)amino]propan-2-yl}amino)-3-oxopropane-1,2-diaminium tetrachloride.

¹H-NMR (400 MHz, DMSO-d₆): δ=0.22 (s, 9H), 1.42-1.48 (m, 2H), 1.53-1.58 (m , 2H), 2.53-2.62 (m, 2H), 3.07-3.11(m, 2H), 3.50 (br, 6H), 4.26 (br., 1H), 4.33 (br., 1H), 4.39-4.53 (m, 1H), 7.16-7.36 (m, 4H), 8.40-9.10 (m, 12H) ppm.

### Example 8e

### Tetragadolinium N-{2-[4,7,10-tris(carboxylatomethyl)-1,4,7,10-tetraazacyclododecan-1-yl]propanoyl}glycyl-3-[(N-{2-[4,7,10-tris(carboxylatomethyl)-1,4,7,10-tetraazacyclo-dodecan-1-yl]propanoyl}glycyl)amino]alanyl-N-(4-{3-[(trimethylsilyl)ethynyl]phenyl}-butyl)-3-[(N-{2-[4,7,10-tris(carboxylatomethyl)-1,4,7,10-tetraazacyclo dodecan-1-yl]-propanoyl}glycyl-3-[N-{2-[4,7,10-tris(carboxylatomethyl)-1,4,7,10-tetraazacyclo-dodecan-1-yl]propanoyl}glycyl)amino]alanyl)amino]alaninamide

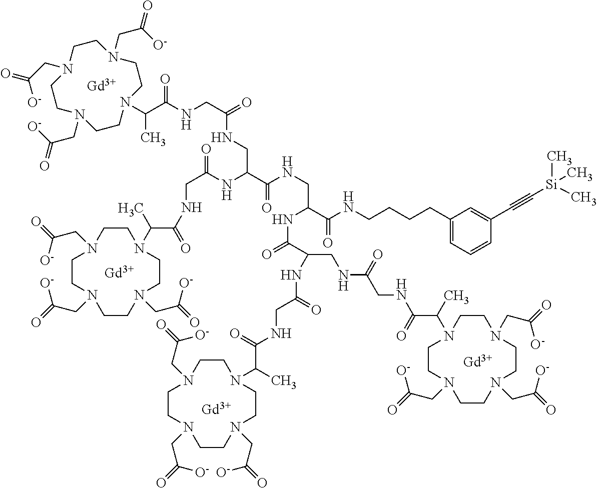

Gadolinium 2,2′,2″-[10-(1-{[2-(4-nitrophenoxy)-2-oxoethyl]amino}-1-oxopropan-2-yl)-1,4,7,10-tetraazacyclododecane-1,4,7-triyl]triacetate (4.62 g, 6.1 mmol) in DMSO (9.0 mL) was added to 3-({(3-{[2,3-diammoniopropanoyl]amino}-1-oxo-1-[(4-{3-[(trimethylsilyl)ethynyl]phenyl}butyl)amino]propan-2-yl}amino)-3-oxopropane-1,2-diaminium tetrachloride (500 mg, 0.77 μmol) and triethylamine (2.6 L, 18.5 mmol) in DMSO (8.0 mL). The mixture was stirred for one hour at 40° C. and 10 hours at 60° C. The mixture was condensed under vacuum, diluted with water adjusted to pH 7 by aqueous sodium hydroxide and low molecular weight components were separated via ultrafiltration (cellulose acetate membrane, lowest NMWL 1000 g/mol, Millipore). The retentate was collected to yield 3.08 g of Tetragadolinium N-{2-[4,7,10-tris(carboxylatomethyl)-1,4,7,10-tetraazacyclododecan-1-yl]propanoyl}glycyl-3-[(N-{2-[4,7,10-tris(carboxylatomethyl)-1,4,7,10-tetraazacyclododecan-1-yl]propanoyl}glycyl)amino]alanyl-N-(4-{3-[(trimethylsilyl)ethynyl]phenyl}butyl)-3-[(N-{2-[4,7,10-tris(carboxylatomethyl)-1,4,7,10-tetraazacyclododecan-1-yl]propanoyl}glycyl-3-[(N-{2-[4,7,10-tris(carboxylatomethyl)-1,4,7,10-tetraazacyclododecan-1-yl]propanoyl}glycyl)amino]alanyl)amino]alaninamide.

UPLC (ACN-HCOOH polar): Rt.=1.51 min.

MS (ES⁻): m/e=1473.9 (M−2H)²⁻.

### Example 8f

### Tetragadolinium N-{2-[4,7,10-tris(carboxylatomethyl)-1,4,7,10-tetraazacyclo dodecan-1-yl]propanoyl}glycyl-3-[(N-{2-[4,7,10-tris(carboxylatomethyl)-1,4,7,10-tetraazacyclo-dodecan-1-yl]propanoyl}glycyl)amino]alanyl-N-(4-{3-[(5-{(1S)-2-carboxy-1-[({(3R)-1-[3-(piperidin-4-yl)propanoyl]piperidin-3-yl}carbonyl)amino]ethyl}pyridin-3-yl)ethynyl]-phenyl}butyl)-3-[(N-{2-[4,7,10-tris(carboxylatomethyl)-1,4,7,10-tetraazacyclododecan-1-yl]propanoyl}glycyl-3-[(N-{2-[4,7,10-tris(carboxylatomethyl)-1,4,7,10-tetraazacyclo-dodecan-1-yl]propanoyl}glycyl)amino]alanyl)amino]alaninamide

To a degased solution of (3S)-3-(5-bromopyridin-3-yl)-3-[({(3R)-1-[3-(piperidin-4-yl)propanoyl]piperidin-3-yl}carbonyl)amino]propanoic acid (15 mg, 30 μmol), triethylamine (20 μL, 150 μmol), and TBAF (60 μL) in water (0.1 mL) and acetonitrile (0.3 mL), was added 1.4 mL of a red catalyst solution, prepared by heating palladium(II)acetate (3.4 mg, 15 μmol) with trisodium 3,3′,3″-phosphanetriyltris(4,6-dimethylbenzenesulfonate) (39 mg, 60 μmol) in water (7 mL) for 30 minutes to 80° C. Tetragadolinium N-{2-[4,7,10-tris(carboxylatomethyl)-1,4,7,10-tetraazacyclododecan-1-yl]propanoyl}glycyl-3-[(N-{2-[4,7,10-tris(carboxylatomethyl)-1,4,7,10-tetraazacyclododecan-1-yl]propanoyl}glycyl)amino]alanyl-N-(4-{3-[(trimethylsilyl)ethynyl]phenyl}butyl)-3-[(N-{2-[4,7,10-tris(carboxylatomethyl)-1,4,7,10-tetraaza cyclododecan-1-yl]propanoyl}glycyl-3-[(N-{2-[4,7,10-tris(carboxylatomethyl)-1,4,7,10-tetraazacyclododecan-1-yl]propanoyl}glycyl)amino]alanyl)amino]alaninamide (208 mg, 70 μmol) in water (1.2 mL) and acetonitrile (0.8 mL) was added over two hours at 60° C. The mixture was heated at 60° C. for additional 22 hours, after cooling to room the mixture was condensed and the residue purified by preparative HPLC (C18-YMC ODS AQ-10 μm, acetonitrile in water+0.1% formic acid, 15% to 55%) followed by ultrafiltration (cellulose acetate membrane, lowest NMWL 500 g/mol, Millipore) to yield 9.8 mg of the title compound in the condensed retentate.

UPLC (ACN-HCOOH polar): Rt.=0.96 min.

MS (ES⁻): m/e=1645.9 (M−2H)²⁻

### Example 9

### Tetragadolinium 2,3-bis({2,3-bis[(N-{2-[4,7,10-tris(carboxylatomethyl)-1,4,7,10-tetra azacyclododecan-1-yl]propanoyl}glycyl)amino]propanoyl}amino)-N-(4-{3-[(5-{(1S)-2-carboxy-1-[({(3R)-1-[3-(piperidin-4-yl)propanoyl]piperidin-3-yl}carbonyl)amino]ethyl}pyridin-3-yl)ethyl]phenyl}butyl)propanamide

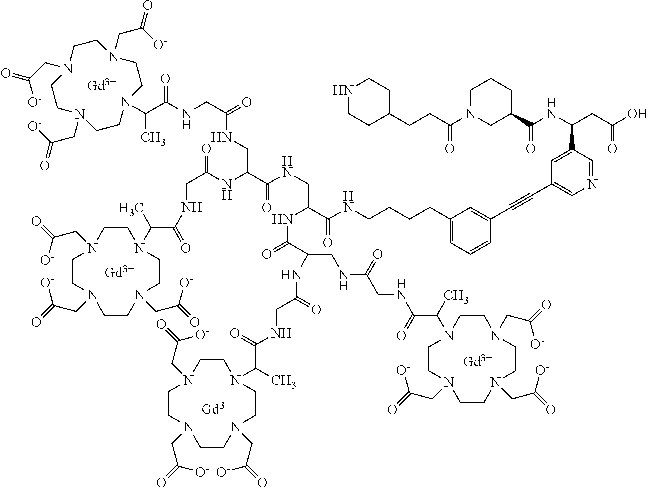

Tetragadolinium 2,3-bis({2,3-bis[(N-{2-[4,7,10-tris(carboxylatomethyl)-1,4,7,10-tetraazacyclo dodecan-1-yl]propanoyl}glycyl)amino]propanoyl}amino)-N-(4-{3-[(5-{(1S)-2-carboxy-1-[({(3R)-1-[3-(piperidin-4-yl)propanoyl]piperidin-3-yl}carbonyl)amino]ethyl}pyridin-3-yl)ethynyl]phenyl}-butyl)propanamide (25 mg, 7.6 μmol) in ethanol (0.11 mL) and water (0.68 mL) was stirred under a hydrogen atmosphere for in the presence of palladium on charcoal (10%, 3.8 mg) for 20 hours. The mixture was diluted with ethanol and water and filtered. The filtrate was condensed under vacuum and the residue purified by preparative HPLC (C18-YMC ODS AQ-10 μm, acetonitrile in water+0.1% formic acid, 1% to 25%) to yield 8.3 mg of the title compound.

UPLC (ACN-HCOOH polar): Rt.=0.84 min.

MS (ES⁻): m/e=1647.8 (M−2H)²⁻.

### Example 10

### Digadolinium N-{2-[4,7,10-tris(carboxylatomethyl)-1,4,7,10-tetraazacyclododecan-1-yl]-propanoyl}glycyl-N-(3-{4-[(5-{(1S)-2-carboxy-1-[({(3R)-1-[3-(piperidin-4-yl)propanoyl]-piperidin-3-yl}carbonyl)amino]ethyl}pyridin-3-yl)ethynyl]phenyl}propyl)-3-[(N-{2-[4,7,10-tris(carboxylatomethyl)-1,4,7,10-tetraazacyclododecan-1-yl]propanoyl}glycyl)-amino]alaninamide

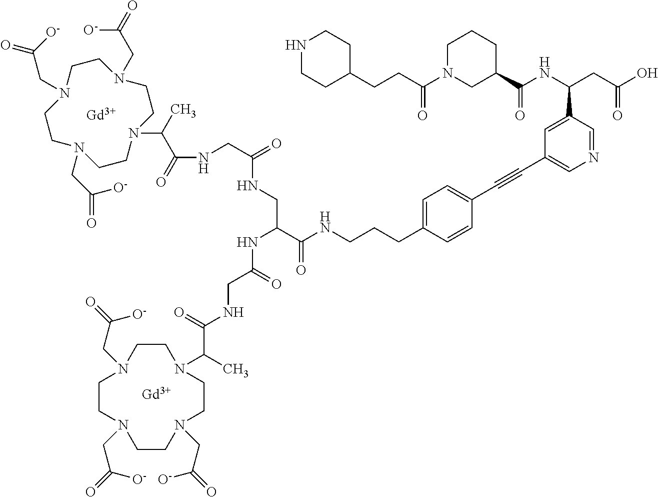

### Example 10a

### 2-[3-(4-Hydroxyphenyl)propyl]-1H-isoindole-1,3(2H)-dione

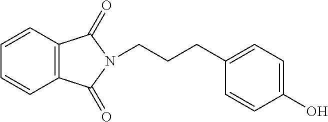 [SMILES: O=C1c2ccccc2C(=O)N1CCCc1ccc(O)cc1]

4-Bromophenol (4.77 g, 27.6 mmol), 2-(prop-2-en-1-yl)-1H-isoindole-1,3(2H)-dione (6.7 g, 19.7 mmol), palladium(II)acetate (44 mg, 0.20 mmol) and tris(2-methylphenyl)phosphane (120 mg, 0.39 mmol) were stirred in acetonitrile (104 mL) and triethylamine (5.5 mL) for 20 hours at 100° C. After concentration an E/Z mixture mixture of 2-[3-(4-hydroxyphenyl)prop-2-en-1-yl]-1H-isoindole-1,3(2H)-dione was obtained, which could be purified by chromatography on silica gel (ethyl acetate in hexane, 10 to 80%) to yield 2.76 g of the alkene intermediate. The 2-[3-(4-hydroxyphenyl)prop-2-en-1-yl]-1H-isoindole-1,3(2H)-dione was solved in methanol (246 mL), water (18 mL) and ethyl acetate (205 mL) and stirred under a hydrogen atmosphere for in the presence of palladium on charcoal (10%, 287 mg) at 40° C. for 6 hours. The reaction mixture was filtered through a path of celite, concentrated under reduced pressure and the residue was purified by chromatography on silica gel (ethyl acetate in hexane, 0 to 60%) to yield 1.69 g of 2-[3-(4-hydroxyphenyl)propyl]-1H-isoindole-1,3(2H)-dione.

¹H-NMR (300 MHz, DMSO-d₆): δ=1.84 (quin, 2H), 2.49 (t, 1H), 3.58 (t, 2H), 6.65 (d, 2H), 7.00 (d, 2H), 7.84 (m, 4H), 9.09 (s, 1H) ppm.

### Example 10b

### 4-[3-(1,3-Dioxo-1,3-dihydro-2H-isoindol-2-yl)propyl]phenyl trifluoromethanesulfonate

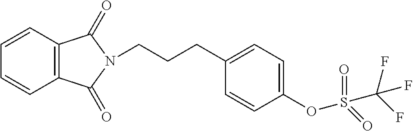 [SMILES: O=C1c2ccccc2C(=O)N1CCCc1ccc(OS(=O)(=O)C(F)(F)F)cc1]

To 2-[3-(4-hydroxyphenyl)propyl]-1H-isoindole-1,3(2H)-dione (5.8 g, 20.6 mmol) in pyridine (43 mL) was added trifluoromethane sulfonic anhydride (4.54 mL, 26.8 mmol) at 0° C. The mixture was stirred for one hour while the mixture warmed to room temperature, a mixture of water and diethyl ether was added, the phases were separated and the aqueous phase was extracted with diethyl ether. The combined organic extracts were washed with 0.5 M hydrochloric acid and dried over sodium sulfate. The solution was concentrated under reduced pressure while toluene was added two times before the end of the distillation and the residue was purified by chromatography on silica gel (ethyl acetate in hexane, 0 to 70%) to yield 7.61 g of 4-[3-(1,3-dioxo-1,3-dihydro-2H-isoindol-2-yl)propyl]phenyl trifluoromethane sulfonate.

¹H-NMR (300 MHz, DMSO-d₆): δ=1.91 (quin, 2H), 2.68 (t, 2H), 3.60 (t, 2H), 7.30 (d, 2H), 7.45 (d, 2H), 7.77-7.90 (m, 4H) ppm.

### Example 10c

### 2-(4-{3-[(Trimethylsilyl)ethynyl]phenyl}propyl)-1H-isoindole-1,3(2H)-dione

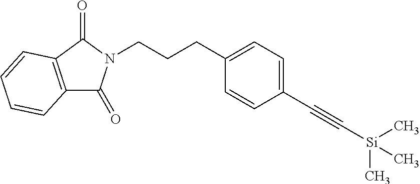 [SMILES: C[Si](C)(C)C#Cc1ccc(CCCN2C(=O)c3ccccc3C2=O)cc1]

To 4-[3-(1,3-dioxo-1,3-dihydro-2H-isoindol-2-yl)propyl]phenyl trifluoromethane sulfonate (7.6 g, 18.4 mmol), dichloropalladium(II)bis(triphenylphosphane) (646 mg, 0.92 mmol), copper iodide (175 mg, 0.92 mmol) and N,N-diisopropylethylamine (16 mL, 92 mmol) in DMF (18 mL) was added ethynyl(trimethyl)silane (15.3 mL, 110 mmol) in DMF (16 mL) over 15 hours at 50° C. After stirring for 27 additional hours at 50° C. a mixture of water and diethyl ether was added, the phases were separated and the aqueous phase was extracted with diethyl ether. Combined organic extracts were concentrated under reduced pressure while toluene was added multiple times at the end of the distillation and the residue was purified by chromatography on silica gel (ethyl acetate in hexane, 0 to 100%) to yield 3.43 g 2-(3-{4-[(trimethylsilyl)ethynyl]phenyl}propyl)-1H-isoindole-1,3(2H)-dione.

¹H-NMR (400 MHz, DMSO-d₆): δ=0.21 (s, 9H), 1.79-1.98 (m, 2H), 2.63 (t, 2H), 3.58 (t, 2H), 7.21 (d, 2H), 7.33 (d, 2H), 7.68-7.94 (m, 4H) ppm.

### Example 10d

### 4-{3-[(Trimethylsilyl)ethynyl]phenyl}propan-1-amine

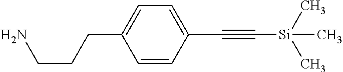 [SMILES: C[Si](C)(C)C#Cc1ccc(CCCN)cc1]

To 2-(3-{4[(trimethylsilyl)ethynyl]phenyl}propyl)-1H-isoindole-1,3(2H)-dione (4.68 g, 13.0 mmol) in THF (105 mL) was added methyl hydrazine (13.8 mL, 259 mmol, additional 7.0 mL 130 mmol after 24 hours) in two portions and the solution was stirred for 41 hours at 40° C. while a precipitate formed. The reaction mixture was concentrated to a volume of 40 mL and filtered at 0° C. The solid was washed with a small amount of cold THF and the combined filtrates concentrated under reduced pressure while toluene was added two times before the end of the distillation to quantitatively yield 4-{3-[(trimethylsilyl)ethynyl]phenyl}butan-1-amine.

¹H-NMR (300 MHz, DMSO-d₆): δ=0.22 (s, 9H), 1.72 (quin, 2H), 2.63 (m, 4H), 4.39 (br. s., 2H), 7.20 (d, 2H), 7.36 (d, 2H) ppm.

### Example 10e

### N²-(Tert-butoxycarbonyl)-3-[(tert-butoxycarbonyl)amino]-N-(3-{4-[(trimethylsilyl)-ethynyl]phenyl}propyl)alaninamide

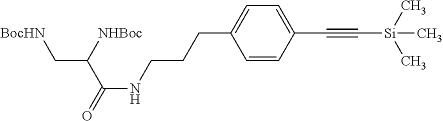 [SMILES: CC(C)(C)OC(=O)NCC(NC(=O)OC(C)(C)C)C(=O)NCCCc1ccc(C#C[Si](C)(C)C)cc1]

4-{3-[(Trimethylsilyl)ethynyl]phenyl}butan-1-amine (0.6 g, 2.6 mmol) in DMF (6 mL) was added to a freshly prepared solution of N-(tert-butoxycarbonyl)-3-[(tert-butoxy-carbonyl)amino]alanine N-cyclohexylcyclohexanamine (0.69 g, 1.4 mmol), N,N-diisopropylethylamine (1.1 mL, 6.5 mmol) and HATU (0.69 g, 1.82 mmol) in DMF (6 mL) at 0° C. After stirring for one hour additional N-(tert-butoxycarbonyl)-3-[(tert-butoxy-carbonyl)amino]alanine N-cyclohexylcyclohexanamine (0.2 g, 0.4 mmol) and HATU (0.2 g, 0.5 mmol) was added, the mixture was filtered cold after three hours, the filtrate condensed, while remaining traces of DMF were distilled in the presence of toluene, and purified by chromatography on amino phase silica gel (ethyl acetate in hexane, 0 to 70%) to yield 0.55 g of N-(tert-butoxycarbonyl)-3-[(tert-butoxycarbonyl)amino]-N-(3-{4-[(trimethylsilyl)ethynyl]phenyl}propyl)alaninamide.

¹H-NMR (300 MHz, DMSO-d₆): δ=0.22 (s, 9H), 1.34 (s, 9H), 1.37 (s, H), 1.66 (quin, 2H), 2.56 (t, 2H), 2.97-3.08 (td, 2H), 3.17 (t, 2H), 3.96 (m, 1H), 6.64 (d, 1H), 6.71 (t, 1H), 7.20 (d, 2H), 7.35 (d, 2H), 7.85 (t, 1H) ppm.

### Example 10f

### 3-Oxo-3-[(3-{4-[(trimethylsilyl)ethynyl]phenyl}propyl)amino]propane-1,2-diaminium dichloride

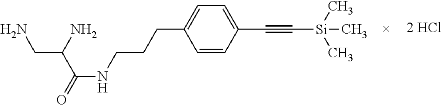 [SMILES: C[Si](C)(C)C#Cc1ccc(CCCNC(=O)C(N)CN)cc1]

N-(Tert-butoxycarbonyl)-3-[(tert-butoxycarbonyl)amino]-N-(3-{4-[(trimethylsilyl)ethynyl]phenyl}propyl)alaninamide (0.55 g, 0.85 mmol) in DMF (1.5 mL) was added to hydrochloric acid in dioxane (4M, 1.5 mL) in a pressure vessels, which were sealed and irradiated in a microwave reactor for 12 minutes at 80° C. The reaction solution was condensed, while remaining traces of DMF were distilled in the presence of toluene. The residue was diluted with 1,4-dioxane (200 mL), DMF (2 mL) and hydrochloric acid in dioxane (4M, 2 mL). The mixture was stirred while a precipitate formed which was collected by filtration to yield 0.32 g of 3-oxo-3-[(3-{4-[(trimethylsilyl)ethynyl]phenyl}propyl)amino]propane-1,2-diaminium dichloride.

¹H-NMR (300 MHz, DMSO-d₆): δ=0.22 (s, 9H), 1.75 (quin, 2H), 2.66 (t, 2H), 3.00-3.22 (m, 2H), 3.26 (m, 2H), 4.25 (t, 1H), 7.25 (d, 2H), 7.38 (d, 2H), 8.62 (br. s., 6H), 8.97 (t, 1H) ppm.

### Example 10g

### Digadolinium N-{2-[4,7,10-tris(carboxylatomethyl)-1,4,7,10-tetraazacyclododecan-1-yl]propanoyl}glycyl-N-(3-{4-[(trimethylsilyl)ethynyl]phenyl}propyl)-3-[(N-{2-[4,7,10-tris(carboxylatomethyl)-1,4,7,10-tetraazacyclododecan-1-yl]propanoyl }glycyl)amino]alaninamide

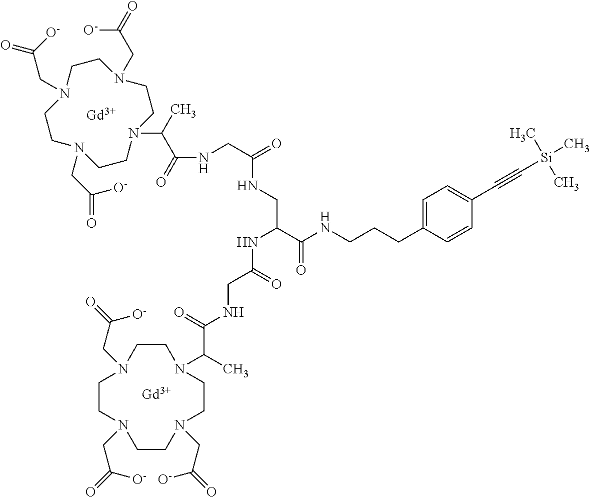

Gadolinium 2,2′,2″-[10-(1-{[2-(4-nitrophenoxy)-2-oxoethyl]amino}-1-oxopropan-2-yl)-1,4,7,10-tetraazacyclododecane-1,4,7-triyl]triacetate (1.74 g, 1.62 mmol) was added to 3-oxo-3-[(3-{4-[(trimethylsilyl)ethynyl]phenyl}propyl)amino]propane-1,2-diaminium dichloride (320 mg, 0.74 μmol) and triethylamine (1.5 mL, 18.5 mmol) in DMF (14 mL). The mixture was stirred for 8 hours at 55° C. The mixture was condensed under vacuum while toluene was added multiple times at the end of the distillation, diluted with water adjusted to pH 7 by aqueous sodium hydroxide and low molecular weight components were separated via ultrafiltration (cellulose acetate membrane, lowest NMWL 1000 g/mol, Millipore). The retentate was collected to yield 0.74 g of digadolinium N-{2-[4,7,10-tris(carboxylatomethyl)-1,4,7,10-tetraazacyclododecan-1-yl]propanoyl}glycyl-N-(3-{4-[(trimethylsilyl)ethynyl]phenyl}propyl)-3-[(N-{2-[4,7,10-tris-(carboxylatomethyl)-1,4,7,10-tetraazacyclododecan-1-yl]propanoyl}glycyl)amino]alaninamide as a mixture of stereoisomers.

UPLC (ACN-HCOOH polar): Rt.=1.63, 1.66, 1.68 min.

MS (ES⁻): m/e=1538.0 (M−H)⁻.

### Example 10g

### Digadolinium N-{2-[4,7,10-tris(carboxylatomethyl)-1,4,7,10-tetraazacyclododecan-1-yl]-propanoyl}glycyl-N-(3-{4-[(5-{(1S)-2-carboxy-1-[({(3R)-1-[3-(piperidin-4-yl)propanoyl]-piperidin-3-yl}carbonyl)amino]ethyl}pyridin-3-yl)ethynyl]phenyl}propyl)-3-[(N-{2-[4,7,10-tris(carboxylatomethyl)-1,4,7,10-tetraazacyclododecan-1-yl]propanoyl}glycyl)amino]alaninamide

To a solution of (3S)-3-(5-bromopyridin-3-yl)-3-[({(3R)-1-[3-(piperidin-4-yl)propanoyl]piperidin-3-yl}carbonyl)amino]propanoic acid (20 mg, 40 μmol), triethylamine (30 μL, 200 μmol), and tetramethyl ammonium flouride (7.5 mg, 80 μmol) in water (140 μL) and acetonitrile (60 μL), was added 1.5 mL of a red catalyst solution, prepared by heating palladium(II)acetate (1.8 mg, 8 μmol) with trisodium 3,3′,3″-phosphanetriyltris(4,6-dimethyl benzenesulfonate) (21 mg, 32 μmol) in water (1.5 mL) for 30 minutes to 80° C. under argon. The mixture was degased by helium and digadolinium N-{2-[4,7,10-tris(carboxylatomethyl)-1,4,7,10-tetraazacyclododecan-1-yl]propanoyl}glycyl-N-(3-{4-[(trimethylsilyl)ethynyl]phenyl}propyl)-3-[(N-{2-[4,7,10-tris(carboxylatomethyl)-1,4,7,10-tetraazacyclododecan-1-yl]propanoyl}glycyl)amino]alaninamide (90 mg, 50 μmol) in water (2 mL) was added over 8 hours at 60° C. The mixture was heated at 60° C. for additional 12 hours, after cooling to room the mixture was condensed and the residue purified by preparative HPLC (C18-YMC ODS AQ-10 μm, acetonitrile in water+0.1% formic acid, 15% to 65%) to yield 12.2 mg of the title compound as a mixture of stereoisomers.

UPLC (ACN-HCOOH polar): Rt.=0.96-0.98 min.

MS (ES⁻): m/e=1883.2 (M−H)⁻.

### Example 11

### Tetragadolinium 2,3-bis({2,3-bis[(N-{2-[4,7,10-tris(carboxylatomethyl)-1,4,7,10-tetraaza-cyclododecan-1-yl]propanoyl}glycyl)amino]propanoyl}amino)-N-(3-{4-[(5-{(1S)-2-carboxy-1-[({(3R)-1-[3-(piperidin-4-yl)propanoyl]piperidin-3-yl}carbonyl)amino]ethyl}-pyridin-3-yl)ethynyl]phenyl}propyl)propanamide

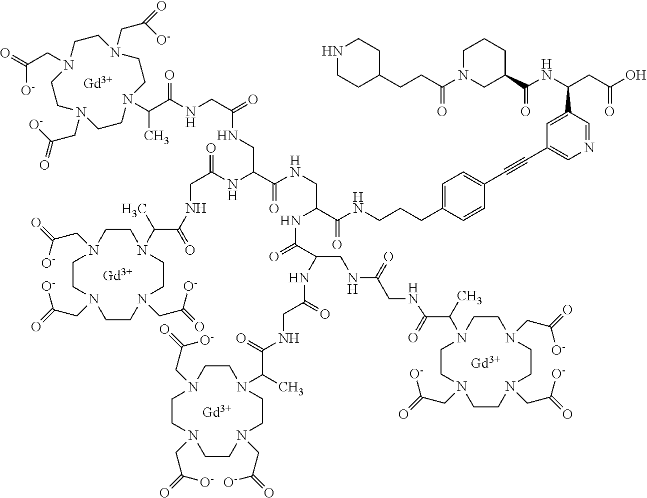

### Example 11a

### N-(Tert-butoxycarbonyl)-3-[(tert-butoxycarbonyl)amino]alanyl-3-({N-(tert-butoxy-carbonyl)-3-[(tert-butoxycarbonyl)amino]alanyl}amino)-N-(3-{4-[(trimethylsilyl)ethynyl]-phenyl}propyl)alanine amide

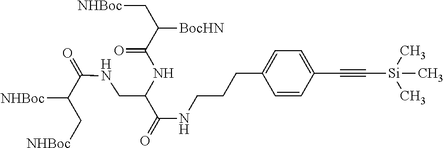 [SMILES: CC(C)(C)OC(=O)NCC(NC(=O)OC(C)(C)C)C(=O)NCC(NC(=O)C(CNC(=O)OC(C)(C)C)NC(=O)OC(C)(C)C)C(=O)NCCCc1ccc(C#C[Si](C)(C)C)cc1]

3-Oxo-3-[(3-{4-[(trimethylsilyl)ethynyl]phenyl}propyl)amino]propane-1,2-diaminium dichloride (600 mg, 1.23 mmol) in DMF (10 mL) and N,N-diisopropylethylamine (1.6 mL) was added to a freshly prepared solution of N-(tert-butoxycarbonyl)-3-[(tert-butoxycarbonyl)amino]-alanine N-cyclohexyl cyclohexanamine (1.4 g, 2.95 mmol) and HATU (1.31 g, 3.44 mmol) in DMF (13.7 mL) and N,N-diisopropylethylamine (2.4 mL) at 20° C. After stirring for two hours and storage for 18 hours at 6° C. the cold mixture was filtrated and the precipitate was washed with ethyl acetate. The filtrate was condensed codestilled with toluene and the residue purified by chromatography on amino phase silica gel (ethyl acetate in hexane, 0 to 100%) to yield 0.55 g of N-(tert-butoxycarbonyl)-3-[(tert-butoxycarbonyl)amino]alanyl-3-({N-(tert-butoxycarbonyl)-3-[(tert-butoxycarbonyl)amino]alanyl}amino)-N-(3-{4[(trimethylsilyl)ethynyl]-phenyl}propyl)alanine amide.

¹H-NMR (400 MHz, DMSO-d₆): δ=0.21 (m, 9H), 1.36 (s, 36H), 1.69 (t, 2H), 2.56 (t, 2H), 3.04-3.26 (m, 8H), 3.96 (br, 2H), 4.22 (br, 1H), 6.40-6.57 (m, 1H), 6.67-6.83 (m, 3H) 7.19 (d, 2H), 7.34 (d, 2H), 7.72-8.01 (m, 2H), 8.11 (t, 1H) ppm.

### Example 11b

### 3-({(3-{[2,3-Diammoniopropanoyl]amino)-1-oxo-1-[(3-(4-[(trimethylsilyl)ethynyl]phenyl}-propyl)amino]propan-2-yl}amino)-3-oxopropane-1,2-diaminium tetrachloride

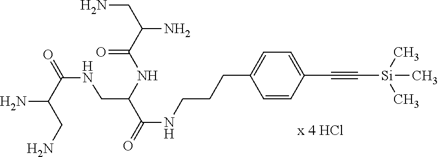 [SMILES: C[Si](C)(C)C#Cc1ccc(CCCNC(=O)C(CNC(=O)C(N)CN)NC(=O)C(N)CN)cc1]

To N-(tert-butoxycarbonyl)-3-[(tert-butoxycarbonyl)amino]alanyl-3-({N-(tert-butoxycarbonyl)-3-[(tert-butoxycarbonyl)amino]alanyl}amino)-N-(3-{4[(trimethylsilyl)ethynyl]phenyl}propyl)alanine amide (0.54 g, 546 μmol) in DMF (4 mL) was added hydrochloric acid in dioxane (4M, 4 mL) The reaction vessel was sealed and irradiated in a microwave reactor for 10 minutes at 80° C. After cooling to room temperature the reaction mixture was slowly added to 1,4-dioxane while stirring. The formed precipitate was collected by filtration to yield 0.20 g of 3-({(3-{[2,3-diammoniopropanoyl]amino}-1-oxo-1-[(3-{4-[(trimethylsilyl)ethynyl]phenyl}propyl) amino]propan-2-yl}amino)-3-oxopropane-1,2-diaminium tetrachloride.

¹H-NMR (400 MHz, DMSO-d₆): δ=0.22 (s, 9H), 1.67-1.81 (m, 2H), 2.56-2.66 (m, 2H), 3.01-3.15 (m, 2H), 3.19-3.46 (m, 6H), 4.20-4.40 (m, 2H), 4.44-4.55 (m, 1H), 7.19-7.28 (m, 2H), 7.33-7.43 (m, 2H), 8.39-8.66 (br. m, 7H), 8.77 (br, 6H), 9.00-9.18 (m, 2H) ppm.

### Example 11c

### Tetragadolinium 2,3-bis({2,3-bis[(N-{2-[4,7,10-tris(carboxylatomethyl)-1,4,7,10-tetra azacyclododecan-1-yl]propanoyl}glycyl)amino]propanoyl}amino)-N-(3-{4-[(trimethyl silyl)ethynyl]phenyl}propyl)propanamide

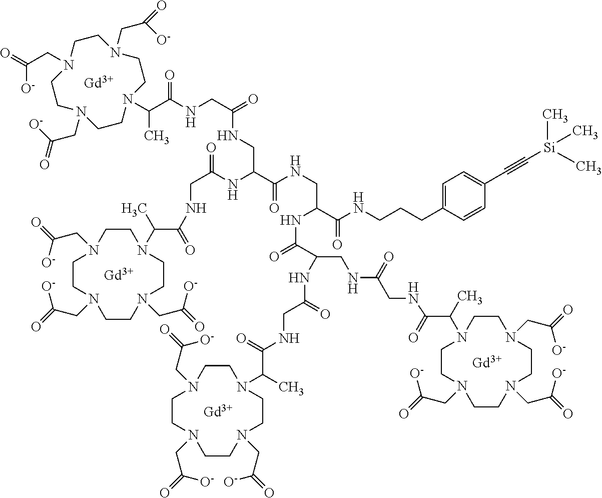

Gadolinium 2,2′,2″-[10-(1-{[2-(4-nitrophenoxy)-2-oxoethyl]amino}-1-oxopropan-2-yl)-1,4,7,10-tetraazacyclododecane-1,4,7-triyl]triacetate (1.89 g, 1.76 mmol) was added as a solid to 3-({(3-{[2,3-diammoniopropanoyl]amino}-1-oxo-1-[(3-{4-[(trimethylsilyl)ethynyl]phenyl}propyl) amino]propan-2-yl}amino)-3-oxopropane-1,2-diaminium tetrachloride (200 mg, 220 μmol) and triethylamine (0.92 mL, 6.6 mmol) in DMSO (6.25 mL). The mixture was stirred for 10 hours at 60° C. The mixture was condensed under vacuum, diluted with water adjusted to pH 7 by aqueous sodium hydroxide and low molecular weight components were separated via ultrafiltration (cellulose acetate membrane, lowest NMWL 1000 g/mol, Millipore). The retentate was collected to yield 1.09 g of tetragadolinium 2,3-bis({2,3-bis[(N-{2-[4,7,10-tris(carboxylatomethyl)-1,4,7,10-tetraazacyclododecan-1-yl]propanoyl}glycyl)amino]propanoyl}amino)-N-(3-{4-[(trimethyl silyl)ethynyl]phenyl}propyl)propanamide.

UPLC (ACN-HCOOH polar): Rt.=1.41 min.

MS (ES⁻): m/e=1466.9 (M−2H)²⁻.

### Example 11d

### Tetragadolinium 2,3-bis({2,3-bis[(N-{2-[4,7,10-tris(carboxylatomethyl)-1,4,7,10-tetraaza cyclododecan-1-yl]propanoyl}glycyl)amino]propanoyl}amino)-N-(3-{4-[(5-{(1S)-2-carboxy-1-[({(3R)-1-[3-(piperidin-4-yl)propanoyl]piperidin-3-yl}carbonyl)amino]ethyl}pyridin-3-yl)ethynyl]phenyl}propyl)propanamide

To a solution of (3S)-3-(5-bromopyridin-3-yl-[({(3R)-1-[3-(piperidin-4-yl)propanoyl]piperidin-3-yl}carbonyl)amino]propanoic acid (20 mg, 40 μmol), triethylamine (30 μL, 200 μmol), and tetramethyl ammonium flouride (7.5 mg, 80 μmol) in water (140 μL) and acetonitrile (60 μL), was added 1.5 mL of a red catalyst solution, prepared by heating palladium(II)acetate (1.8 mg, 8 μmol) with trisodium 3,3′,3″-phosphanetriyltris(4,6-dimethyl benzenesulfonate) (21 mg, 32 μmol) in water (1.5 mL) for 30 minutes to 80° C. under argon. The mixture was degased by helium and tetragadolinium 2,3-bis({2,3-bis[(N-{2-[4,7,10-tris(carboxylatomethyl)-1,4,7,10-tetraazacyclododecan-1-yl]propanoyl}glycyl)amino]propanoyl}amino)-N-(3-{4-[(trimethylsilyl)ethynyl]phenyl}propyl)propanamide (395 mg, 40 μmol) in water (2.0 mL) was added over 8 hours at 60° C. The mixture was heated at 60° C. for additional 12 hours, after cooling to room the mixture was condensed and the residue purified by preparative HPLC (C18-YMC ODS AQ-10 μm, acetonitrile in water+0.1% formic acid, 1% to 45%) to yield 5.1 mg of the title compound.

UPLC (ACN-HCOOH polar): Rt.=0.87 min.

MS (ES⁻): m/e=1638.7 (M−2H)²⁻.

Reference Compound

### (3S)-3-[({(3R)-1-[3-(Piperidin-4-yl)propanoyl]piperidin-3-yl}carbonyl)amino]-3-{6-[³H]-pyridin-3-yl}propanoic acid

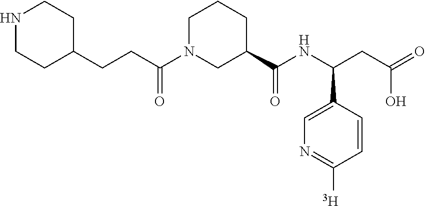 [SMILES: [3H]c1ccc([C@H](CC(=O)O)NC(=O)[C@@H]2CCCN(C(=O)CCC3CCNCC3)C2)cn1]

(3S)-3-(6-Bromopyridin-3-yl)-3-{[(3R)-1-(3-piperidin-4-yl-propanoyl)piperidine-3-carbonyl]amino}propanoic acid (1.85 mg, 3.73 μmol) was dissolved in a mixture of DMF (500 μL) and triethylamine (25 μL). To this solution palladium on charcoal (20%) (6.45 mg) was added and the mixture was connected to a tritium manifold to tritiate over night with tritium gas. Afterwards the reaction mixture was 3 times cryostatically evaporated in the manifold. The obtained crude product was purified on a semi prep HPLC (Kromasil 100 C8 5 μm (250×4.6 mm), eluent: 35 mM ammonia/methanol, flow: 1 mL/min). The collected fraction contained 2061 MBq (S)-3-{5-3H-pyridin-3-yl}-3-{[(R)-1-(3-piperidin-4-yl-propanoyl)piperidin-3-carbonyl]amino}propanoic acid (radiochemical yield: 12.6%; radiochemical purity: 98%; specific activity: 7.81 Ci/mmol).

### Example 12

### Affinities of Investigated Compounds Towards Human GPIIb/IIIa Receptors

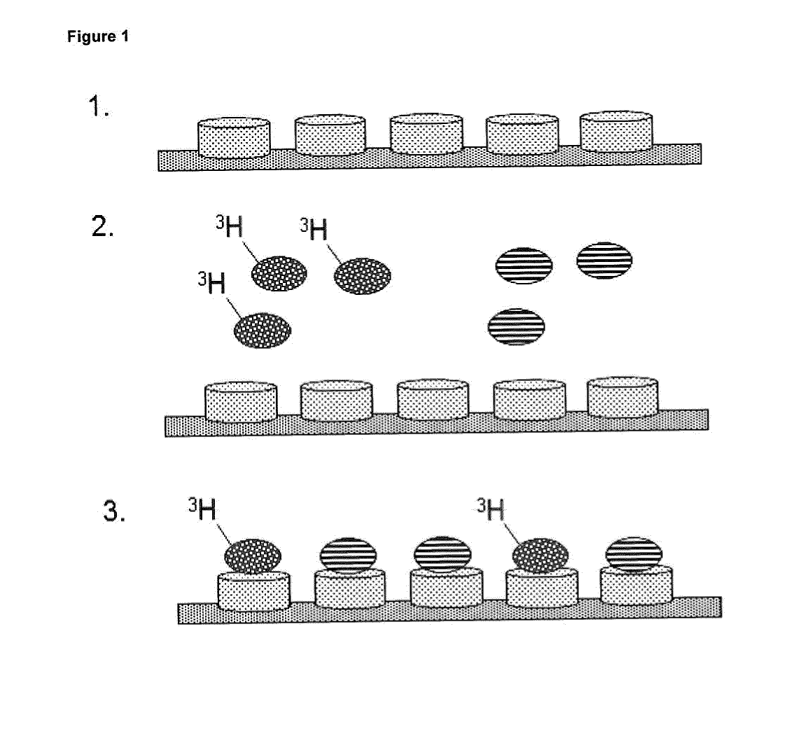

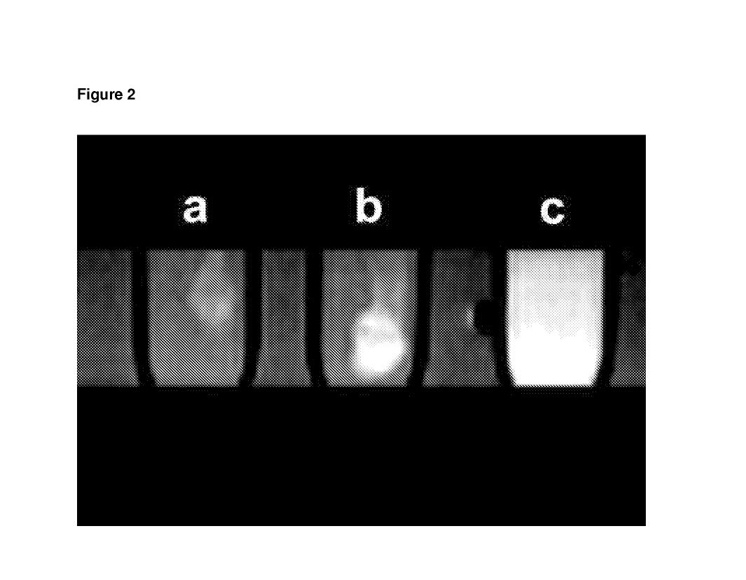

The procedure of the used GPIIb/IIIa affinity assay is schematically demonstrated in FIG. 1. Purified human glycoprotein IIb/IIIa (20 mM Tris-HCl, 0.1 M NaCl, 0.1% Triton X-100, 1 mM CaCl₂, 0.05% NaN₃, 50% Glycerol, pH 7.4) was purchased from Enzyme Research Laboratories Inc. (South Bend, Ind.). The GPIIb/IIIa receptor was diluted in phosphate-buffered saline (Dulbecco's Phosphate Buffered Saline (D-PBS (+)) with calcium and magnesium, GIBCO®, Invitrogen) with 0.01% bovine serum albumin (albumin from bovine serum—lyophilized powder, 96%, Sigma).

The GPIIb/IIIa receptor was immobilized 48 hours at least (100 μL per well, 48 to maximum 96 hours) on a 96-well solid plate (Immuno Plate MaxiSorp™, Nunc, Roskilde, Denmark) at 277 K to 280 K and at a concentration of 0.1 μg per well to 1 μg per well. As negative control one row of the plate (n=8) was incubated just with 2% bovine serum albumin (200 μL per well, albumin from bovine serum—lyophilized powder, ≧96%, Sigma, diluted in D-PBS (+)).

After washing three times with the wash buffer (230 μL per well, Dulbecco's Phosphate Buffered Saline (D-PBS (−)) contains no calcium or magnesium, GIBCO®, Invitrogen) residual exposed plastic and unspecific binding sites were blocked by incubating the plate with a special blocking solution (200 μL per well, Roti®-Block, Car Roth GmbH Co KG, Karlsruhe) containing 2% bovine serum albumin (Albumin from bovine serum—lyophilized powder, ≧96%, Sigma) 1 hour at room temperature.

After washing three times with the wash buffer 50 μL of tritiated reference compound (60 nM, ³H-labeled compound) and 50 μL of novel compound (inhibitor) were simultaneously added to each well and incubated for 1 hour at room temperature. Several concentrations of each novel inhibitor (0.1, 1, 2, 5, 10, 20 50, 100, 200, 500, 1000, 2000, 5000, 10000 and 20000 nM) were investigated. At each concentration of inhibitor a fourfold determination was performed. The results for the examined inhibitors are summarized in table 1.

The maximum value of tritiated reference compound was determined without addition of inhibitor (n=8). To exclude unspecific binding of ³H— reference compound wells without glycoprotein receptors were used as negative controls (n=12, identically treated just without GPIIb/IIIa receptors).

After one hour the plate was washed three times with phosphate-buffered saline (200 μL per well, Dulbecco's Phosphate Buffered Saline (D-PBS (+)), GIBCO®, Invitrogen). Following 140 μL of liquid scintillation cocktail (MicroScint™ 40 aqueous, Perkin Elmer) was added to each well. After 15 min at room temperature the plates were measured at the microplate scintillation counter (TopCount NXT v2.13, Perkin Elmer, Packard Instrument Company).

FIG. 1 shows a schematic diagram of GPIIb/IIIa assay. In the first step human glycoprotein IIb/IIIa, which is purified from human platelets, was immobilized on a 96-well solid plate. After 48 hours at least the plates were washed and the unspecific binding sites were blocked with Roti®-Block. In the next step, the plates were simultaneously incubated with a tritium labeled reference compound and the novel small molecule compound (inhibitor). The higher the affinity of the inhibitor, the smaller is the bound fraction of reference compound. The fraction of tritiated reference compound, which is not displaced by inhibitor, was measured at a microplate scintillation counter. The higher the affinity of the inhibitor, the smaller is the bound fraction of tritium-labeled reference compound. By means of this assay the affinities (IC₅₀ values) could be determined. The studies described above indicate that compounds of formula (I) are useful as contrast agents for the imaging of thrombi. The results are summarized in table 1.

TABLE 1
Binding affinity of compounds towards human GP IIb/IIIa receptor.

| | IC₅₀ human |
|---|---|
| Example | [nM] |
| 1 | 29 |
| 2 | 24 |
| 3 | 13 |
| 4 | 25 |
| 5 | 25 |
| 6 | 103 |
| 7 | 263 |
| 8 | 21 |
| 9 | 32 |
| 10 | 16 |
| 11 | 26 |

### Example 13

Relaxivity Measurements

Relaxivity Measurements

Relaxivity measurements at 1.41 T were performed using a MiniSpec mq60 spectrometer (Bruker Analytik, Karlsruhe) operating at a resonance frequency of 60 MHz and a temperature of 37° C. The T₁ relaxation times were determined using the standard inversion recovery method. The T₂ measurements were done by using the Carr-Purcell-Meiboom-Gill (CPMG) pulse sequence. All measurements were done at concentrations between 0.05 mM and 1 mM of Gd in water and plasma.

The relaxivities rᵢ (where i=1, 2) were calculated on the basis of the measured relaxation rates Rᵢ in water and plasma:

Rᵢ=Rᵢ₍₀₎+rᵢ[C_{Gd}],

where Rᵢ₍₀₎ represent the relaxation rate of the respective solvent and C_{Gd} the concentration of the compound normalized to the Gadolium. The results are summarized in table 2.

TABLE 2
Relaxivities of investigated compounds (normalized to Gd) in water and
plasma at 1.41 T and 37° C. [L mmol⁻¹ s⁻¹]

| Example | r₁ water | r₂ water | r₁ plasma | r₂ plasma |
|---|---|---|---|---|
| 5 | 8.0 | 9.1 | 9.9 | 14.2 |
| 6 | 8.5 | 10.9 | 10.3 | 11.0 |
| 7 | 10.6 | 12.5 | n.d.* | n.d.* |
| 8 | 12.6 | 14.1 | 14.5 | 14.2 |

*not determined

### Example 14

### Binding of Investigated Compounds to Human Activated Platelets

For each experiment fresh blood was taken from a volunteer using 10 mL citrate-tubes (Sarstedt S-Monovette 02.1067.001, 10 mL, Citrate 3.13%). The 10 mL citrate-tubes were carefully inverted 10 times to mix blood and anticoagulant. The tubes were stored in an incubator at a temperature of 37° C. until centrifugation (Heraeus miniTherm CTT with integrated rotation- and turning device, turning speed: 19 rotations per minute, Heraeus Instruments GmbH, Hanau/Germany).

For plasma preparation tube centrifugation was carried out for 15 minutes at 1811 g at room temperature (Eppendorf, Centrifuge 5810R). Blood was centrifuged 15 minutes at 201 g at room temperature to produce platelet-rich plasma. The tubes were stored for 30 min at room temperature to get a better separation. The separated platelet-rich plasma was finally centrifuged for further 3 min at 453 g to remove the remaining erythrocytes. The platelet-rich plasma was activated using a final concentration of 5 μM Adenosindiphosphate (ADP, Sigma). The activated platelet-rich plasma was incubated 20 minutes with different concentrations of gadolinium-labeled compound and subsequently was centrifuged 3 minutes at 1360 g. 20 μL of incubation solution was taken to determine the concentration (n=3). The pellet was resuspended and washed two times with at least 750 μL plasma and subsequently was redispered in 750 μL plasma and 50 μL calciumchloride (50 μL 2%). The gadolinium concentration of supernatant and pellet was determined using an inductively coupled plasma mass spectrometry (ICP-MS Agilent 7500a).

The results for incubation concentration of 1 μM gadolinium-labeled compound are summarized in table 3 (in comparison to Gadovist® standard dose: 100 μmol Gd/kg body weight).

TABLE 3
Binding of investigated compounds to human activated platelets

| | Incubation | Concentration | |
|---|---|---|---|
| | concentration | within platelet | pellet/ |
| Example | [μM molecule] | pellet [μM Gd] | supernatant |
| 2 | 1 | 8.1 ± 0.6** | n.d.* |
| | | (n = 5) | |
| 3 | 1 | 8.3 ± 2.1** | n.d.* |
| | | (n = 5) | |
| 4 | 1 | 3.8 ± 0.2 | n.d.* |
| | | (n = 3) | |
| 5 | 1 | 3.9 ± 0.4 | 123 ± 16 (n = 3) |
| | | (n = 3) | |
| 6 | 1 | 6.8 ± 0.7 | n.d.* |
| | | (n = 3) | |
| 7 | 1 | 6.4 ± 0.3 | n.d.* |
| | | (n = 3) | |
| 8 | 1 | 26.7 ± 1.5 | 254 ± 26 (n = 3) |
| | | (n = 3) | |
| 9 | 0.8 | 21.1 ± 9.6 | n.d. |
| | | (n = 3) | |
| 10 | 1 | 13.9 ± 0.3 | n.d. |
| | | (n = 3) | |
| 11 | 1 | 19.9 ± 0.9 | n.d. |
| | | (n = 3) | |

*not determined
**was not washed

### Example 15

### Magnetic Resonance Imaging

The MRI imaging experiments were done with platelet-rich plasma. The preparation of platelet-rich plasma using fresh blood is described in L K Jennings et. al. Blood 1986 1, 173-179 but modified with regard to centrifugation procedure. Briefly, fresh blood was taken from a volunteer using 10 mL citrate-tubes (Sarstedt S-Monovette 02.1067.001, 10 mL, Citrate 3.13%). The 10 mL citrate-tubes were carefully inverted 10 times to mix blood and anticoagulant. The blood samples were centrifuged 15 minutes at 110 g at room temperature (Eppendorf, Centrifuge 5810R). The tubes were stored for 30 min at room temperature to get a better separation. The separated plasma fraction was centrifuged 3 minutes at 240 g at room temperature to remove remaining erythrocytes. The erythrocyte pellet was eliminated. The platelets in the supernatant were activated using a final concentration of 5 μmol/L Adenosindiphosphate (ADP, Sigma).

The activated platelet-rich plasma solution was incubated 20 minutes at 37° C. with example 8 achieving a final concentration of 10 μmol substance/L. After incubation the samples were centrifuged 3 minutes at 720 g. The supernatant was eliminated and the pellet was washed with 750 μL human plasma three times by repeated redispersing and subsequent centrifugation. In the last washing step Calciumchlorid (70 μL 2%) was added to human plasma to induce platelet aggregation. After 40 min the resulting in vitro platelet-rich thrombi were fixed in 2.0 mL tubes (2.0 mL Eppendorf microcentrifuge tubes) and magnetic resonance imaging in human plasma was performed at room temperature.

The images were performed using a clinical 1.5T system (Siemens Avanto) equipped with a small extremity coil. A T₁-weighted 3D turbo spin echo sequence (3D TSE) with a repetition time (TR) of 1050 ms and an echo-time of 9.1 ms and a turbo factor of 25 was used. The 3D block contains 18 slices each witch a slice thickens of 0.6 mm. The spatial resolution of the 3D TSE sequence was 0.5×0.5×0.6 mm3 with an image matrix of 256×172×18 pixel. The number of signal averages was 16 with a resulting total acquisition time of 17 min and 41 seconds.

The magnetic resonance imaging results are depicted in FIG. 2. In FIG. 2a a control in vitro platelet-rich thrombus without the addition of a contrast agent is shown. The signal intensity of the in vitro thrombus in FIG. 2a is slightly higher than the surrounding medium but clearly lower than the signal of the in vitro thrombus which is incubated with example 8 as depicted in FIG. 2b. In FIG. 2c the incubation solution with a final concentration of 10 μmol substance/L of example 8 in human plasma is represented. The signal intensity of the incubation solution (FIG. 2c) is higher than the surrounding human plasma medium in the in vitro platelet-rich control thrombi sample 2a and in sample 2b. The thrombi in FIG. 2b is incubated 20 min with the solution depicted in 2c. After 20 min incubation period the thrombi in FIG. 2b was washed with plasma solution three times. The signal intensity of the incubated in vitro thrombus in FIG. 2b shows a clearly higher signal than the control thrombi in FIG. 2a.

1. A compound of general formula (I): 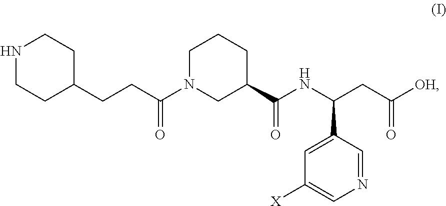
in which:
X represents a group selected from the group consisting of: 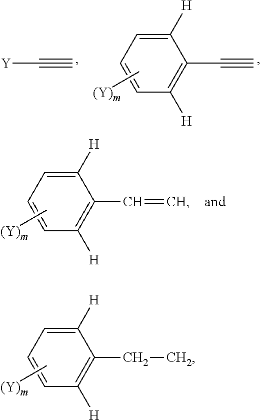
in which group:
Y represents a: 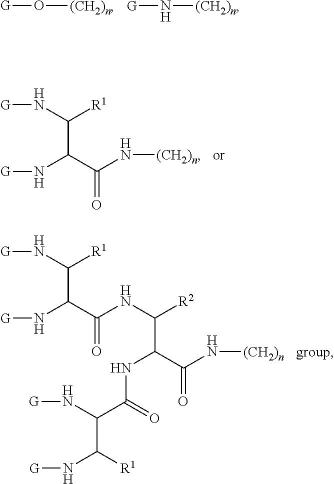
in which groups:
R¹ represents Hydrogen, Methyl, Ethyl, Propyl or iso-Propyl;
R² represents Hydrogen, Methyl, Ethyl, Propyl or iso-Propyl; and
G represents a: 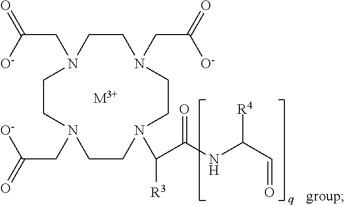
in which:
R³ represents Hydrogen, Methyl, Ethyl, Propyl, iso-Propyl or Benzyl;
R⁴ represents Hydrogen, Methyl, Ethyl, Propyl, iso-Propyl or Benzyl; and
M represents Praseodymium, Neodymium, Samarium, Ytterbium, Gadolinium, Terbium, Dysprosium, Holmium or Erbium;
wherein
m represents 1 or 2;
n represents an integer of 2, 3, 4, 5 or 6; and
q represents 0 or 1;
or a stereoisomer, a tautomer, an N-oxide, a hydrate, a solvate, or a salt thereof, or a mixture of same.

2. A compound of general formula (I), wherein: 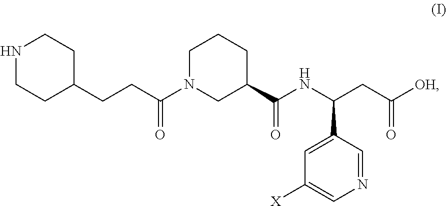
X represents a group selected from the group consisting of: 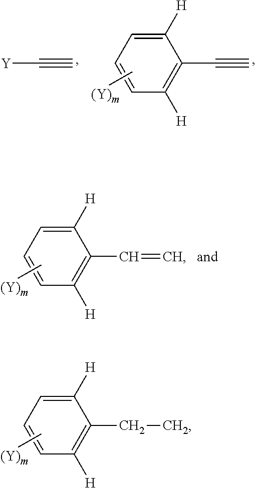
in which group:
Y represents a: 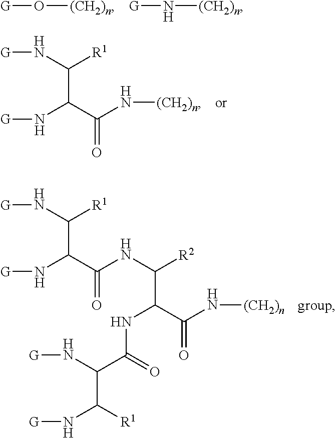
in which groups:
R¹ represents Hydrogen, Methyl, Ethyl, Propyl or iso-Propyl;
R² represents Hydrogen, Methyl, Ethyl, Propyl or iso-Propyl; and
G represents a: 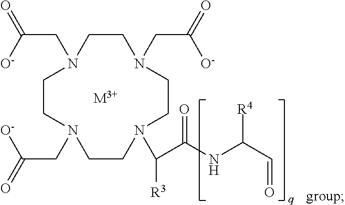
in which:
R³ represents Hydrogen, Methyl, Ethyl, Propyl, iso-Propyl or Benzyl;
R⁴ represents Hydrogen, Methyl, Ethyl, Propyl, iso-Propyl or Benzyl; and
M represents Gadolinium;
wherein
m represents 1 or 2;
n represents an integer of 2, 3, 4, 5 or 6; and
q represents 0 or 1;
or a stereoisomer, a tautomer, an N-oxide, a hydrate, a solvate, or a salt thereof, or a mixture of same.

3. The compound according to claim 1, wherein:
X represents a group selected from the group consisting of: 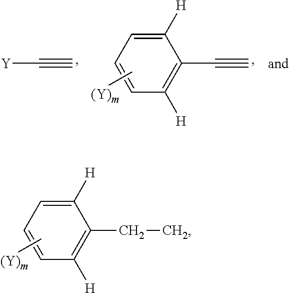
in which group:
Y represents a: 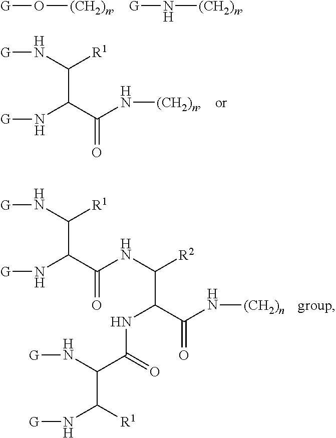
in which groups:
R¹ represents Hydrogen or Methyl;
R² represents Hydrogen or Methyl; and
G represents a: 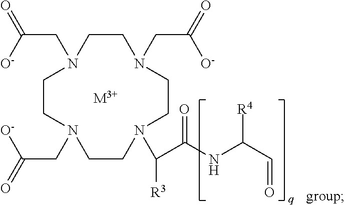
in which:
R³ represents Hydrogen or Methyl;
R⁴ represents Hydrogen or Methyl; and
M represents Gadolinium;
wherein
m represents 1 or 2;
n represents an integer of 2, 3, 4, 5 or 6; and
q represents 0 or 1;
or a stereoisomer, a tautomer, an N-oxide, a hydrate, a solvate, or a salt thereof, or a mixture of same.

4. The compound according to claim 1, wherein:
X represents a group selected from the group consisting of: 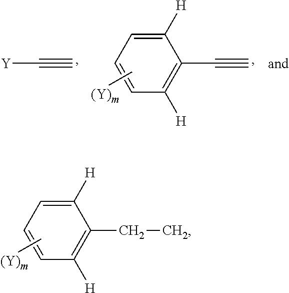
in which group:
Y represents a: 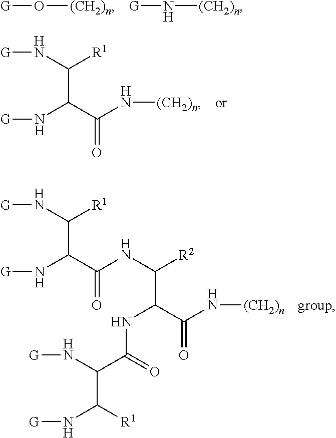
in which groups:
R¹ represents Hydrogen;
R² represents Hydrogen; and
G represents a: 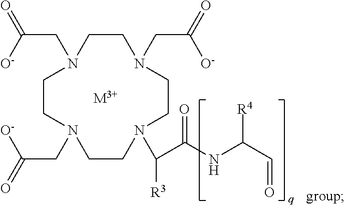
in which:
R³ represents Methyl;
R⁴ represents Hydrogen; and
M represents Gadolinium;
wherein
m represents 1 or 2;
n represents an integer of 2, 3, 4, 5 or 6; and
q represents 1;
or a stereoisomer, a tautomer, an N-oxide, a hydrate, a solvate, or a salt thereof, or a mixture of same.

5. The compound according to claim 1, wherein the compound has a structure selected from the group consisting of:
Gadolinium 2,2′,2″-(10-{(2S)-1-[(2-{[4-(5-{(1S)-2-carboxy-1-[({(3R)-1-[3-(piperidin-4-yl)-propanoyl]piperidin-3-yl}carbonyl)amino]ethyl}pyridin-3-yl)but-3-yn-1-yl]oxy}-2-oxoethyl)-amino]-1-oxopropan-2-yl}-1,4,7,10-tetraazacyclododecane-1,4,7-triyl)triacetate;
Gadolinium 2,2′,2″-(10-{(2S)-1-[(2-{[4-(5-{(1S)-2-carboxy-1-[({(3R)-1-[3-(piperidin-4-yl)-propanoyl]piperidin-3-yl}carbonyl)amino]ethyl}pyridin-3-yl)but-3-yn-1-yl]amino}-2-oxoethyl)-amino]-1-oxopropan-2-yl}-1,4,7,10-tetraazacyclododecane-1,4,7-triyl)triacetate;
Gadolinium 2,2′,2″-{10-[(2S)-1-({2-[(6-{4-[(5-{(1S)-2-carboxy-1-[({(3R)-1-[3-(piperidin-4-yl)-propanoyl]piperidin-3-yl}carbonyl)amino]ethyl}pyridin-3-yl)ethynyl]phenyl}hexyl)amino]-2-oxoethyl}amino)-1-oxopropan-2-yl]-1,4,7,10-tetraazacyclododecane-1,4,7-triyl}triacetate;
Gadolinium 2,2′,2″-{10-[(2S)-1-({2-[(6-{4-[(5-{(1S)-2-carboxy-1-[({(3R)-1-[3-(piperidin-4-yl)-propanoyl]piperidin-3-yl}carbonyl)amino]ethyl}pyridin-3-yl)ethyl]phenyl}hexyl)amino]-2-oxo-ethyl}amino)-1-oxopropan-2-yl]-1,4,7,10-tetraazacyclododecane-1,4,7-triyl}triacetate;
Gadolinium 2,2′,2″-{10-[(2S)-1-({2-[(6-{3-[(5-{(1S)-2-carboxy-1-[({(3R)-1-[3-(piperidin-4-yl)-propanoyl]piperidin-3-yl}carbonyl)amino]ethyl}pyridin-3-yl)ethynyl]phenyl}butyl)amino]-2-oxoethyl}amino)-1-oxopropan-2-yl]-1,4,7,10-tetraazacyclododecane-1,4,7-triyl}triacetate;
Digadolinium 2,2′,2″,2′″,2″″,2′″″-({5-[(5-{(1S)-2-carboxy-1-[({(3R)-1-[3-(piperidin-4-yl)-propanoyl]piperidin-3-yl}carbonyl)amino]ethyl}pyridin-3-yl)ethynyl]-1,3-phenylene}bis[butane-4,1-diylimino(2-oxoethane-2,1-diyl)imino(1-oxopropane-1,2-diyl)-1,4,7,10-tetraazacyclododecane-10,1,4,7-tetrayl])hexaacetate;
Tetragadolinium 2,2′,2″,2′″,2″″,2′″″,2″″″,2′″″″,2″″″″,2′″″″″,2″″″″″,2′″″″″″-({5-[(5-{(1S)-2-carboxy-1-[({(3R)-1-[3-(piperidin-4-yl)propanoyl]piperidin-3-yl}carbonyl)amino]ethyl}pyridin-3-yl)-ethynyl]-1,3-phenylene}bis[butane-4,1-diylcarbamoyl(3,6,11,14-tetraoxo-4,7,10,13-tetra-azahexadecane-8,2,15-triyl)di-1,4,7,10-tetraazacyclododecane-10,1,4,7-tetrayl])dodecaacetate;
Tetragadolinium N-{2-[4,7,10-tris(carboxylatomethyl)-1,4,7,10-tetraazacyclododecan-1-yl]-propanoyl}glycyl-3-[(N-{2-[4,7,10-tris(carboxylatomethyl)-1,4,7,10-tetraazacyclododecan-1-yl]propanoyl}glycyl)amino]alanyl-N-(4-{3-[(5-{(1S)-2-carboxy-1-[({(3R)-1-[3-(piperidin-4-yl)-propanoyl]piperidin-3-yl}carbonyl)amino]ethyl}pyridin-3-yl)ethynyl]phenyl}butyl)-3-[(N-{2-[4,7,10-tris(carboxylatomethyl)-1,4,7,10-tetraazacyclododecan-1-yl]propanoyl}glycyl-3-[(N-{2-[4,7,10-tris(carboxylatomethyl)-1,4,7,10-tetraazacyclododecan-1-yl]propanoyl}glycyl)amino]alanyl)amino]alaninamide;
Tetragadolinium 2,3-bis({2,3-bis[(N-{2-[4,7,10-tris(carboxylatomethyl)-1,4,7,10-tetraazacyclo dodecan-1-yl]propanoyl}glycyl)amino]propanoyl}amino)-N-(4-{3-[(5-{(1S)-2-carboxy-1-[({(3R)-1-[3-(piperidin-4-yl)propanoyl]piperidin-3-yl}carbonyl)amino]ethyl}pyridin-3-yl)ethyl]phenyl}butyl)propanamide;
Digadolinium N-{2-[4,7,10-tris(carboxylatomethyl)-1,4,7,10-tetraazacyclododecan-1-yl]propanoyl}glycyl-N-(3-{4-[(5-{(1S)-2-carboxy-1-[({(3R)-1-[3-(piperidin-4-yl)propanoyl]piperidin-3-yl}carbonyl)amino]ethyl}pyridin-3-yl)ethynyl]phenyl}propyl)-3-[(N-{2-[4,7,10-tris(carboxylato methyl)-1,4,7,10-tetraazacyclododecan-1-yl]propanoyl}glycyl)amino]alaninamide; and
Tetragadolinium 2,3-bis({2,3-bis[(N-{2-[4,7,10-tris(carboxylatomethyl)-1,4,7,10-tetraazacyclo dodecan-1-yl]propanoyl}glycyl)amino]propanoyl}amino)-N-(3-{4-[(5-{(1S)-2-carboxy-1-[({(3R)-1-[3-(piperidin-4-yl)propanoyl]piperidin-3-yl}carbonyl)amino]ethyl}pyridin-3-yl)ethynyl]phenyl}propyl)propanamide.

6. A method for diagnostic imaging comprising:
administering to a patient a compound of general formula (I) according to claim 1.

7. (canceled)

8. A diagnostic agent comprising one or more compounds of general formula (I) according to claim 1 or mixtures thereof.

9. The diagnostic agent of claim 8 wherein the diagnostic agent is used for imaging thrombi.

10. A method of imaging body tissue in a patient, comprising:
administering to the patient an effective amount of one or more compounds of general formula (I) according to claim 1 in a pharmaceutically acceptable carrier, and
subjecting the patient to a magnetic resonance imaging tomography procedure.
